(19) United States
(12) Patent Application Publication
(10) Pub. No.: US 2024/0360233 A1
(43) Pub. Date: Oct. 31, 2024

(54) ANTI-TSLPR (CRLF2) ANTIBODIES

(71) Applicants: BOARD OF REGENTS, THE UNIVERSITY OF TEXAS SYSTEM, Austin, TX (US); XENCOR, INC., Pasadena, CA (US)

(72) Inventors: Dongxing ZHA, Houston, TX (US); Marina KONOPLEVA, Houston, TX (US); Nitin JAIN, Houston, TX (US); Melinda G. SMITH, Houston, TX (US); Ze TIAN, Houston, TX (US); Jason K. ALLEN, Houston, TX (US); Chunhua SHI, Houston, TX (US); Amin AL-SHAMI, Houston, TX (US); Alex NISTHAL, Monrovia, CA (US); John R. DESJARLAIS, Pasadena, CA (US); Seung CHU, Upland, CA (US); Erik PONG, Monrovia, CA (US)

(21) Appl. No.: 18/687,002

(22) PCT Filed: Aug. 27, 2022
(86) PCT No.: PCT/US2022/075552

§ 371 Date: Feb. 27, 2024

Related U.S. Application Data
(60) Provisional application No. 63/366,529, filed on Jun. 16, 2022.
(60) Provisional application No. 63/268,311, filed on Feb. 21, 2022.
(60) Provisional application No. 63/237,857, filed on Aug. 27, 2021.

(51) Int. Cl. C07K 16/28; A61K 39/00; A61P 35/02

(52) U.S. Cl. CPC C07K 16/2866; A61P 35/02; C07K 16/2809; A61K 2039/505; C07K 2317/31; C07K 2317/52; C07K 2317/55; C07K 2317/565; C07K 2317/622; C07K 2317/732

(57) ABSTRACT

The present disclosure provides antibodies and antigen-binding domain compositions capable of binding to thymic stromal lymphopoietin receptor (TSLPR). Also provided are methods of using such antibodies for the treatment of TSLPR-associated cancers (e.g., B-Cell Acute Lymphoblastic Leukemia (B-ALL)). In embodiments, the antibodies are heterodimeric antibodies that bind TSLPR and CD3 epsilon (i.e., anti-TSLPR x anti-CD3e) antibodies.

### BACKGROUND

The thymic stromal lymphopoietin receptor (TSLPR), a heterodimeric receptor complex comprised of a TSLPR subunit (encoded by the CRLF2 gene) and a CD127 subunit binds the cytokine thymic stromal lymphopoietin (TSLP). Overexpression of the TSLPR has been identified in 5% to 15% of children and adults with B-Cell Acute Lymphoblastic Leukemia (B-ALL) and is associated with poor prognosis. The TSLPR signaling pathway has been identified as biologically important for Acute Lymphoblastic Leukemia (ALL) blasts. Recent comprehensive genomic analyses of high-risk B-ALL cases demonstrated that CRLF2 rearrangements in ALL are frequently associated with a gene expression profile highly similar to that of Philadelphia chromosome (Ph)-positive ALL, but without the BCR-ABL1 fusion. About 20% of B-cell ALL patients have “Philadelphia chromosome-like” (Ph-like) gene expression profiles and about half of Ph-like leukemias are CRLF2-positive. Patients with Ph-like leukemias respond poorly to conventional chemotherapy and have high rates of relapse. Therefore, new treatment modalities for Ph-like B-ALL are needed.

T cell immune response is an important part of the human immune system, and thus, construction of antibodies that bind to T cells and tumor cell surface antigens can activate inactive T cells to kill a tumor target.

Although malignant B cells have been targeted with antibodies that bind, e.g., CD19 and CD3 on T cells, clinically meaningful remission rates have not been achieved, potentially because CD19 is downregulated on B-ALL cells over time. Further, CD19 is expressed on normal B cells and bispecific CD19 x CD3 antibodies have been shown to lead to depletion of normal B cells. Therefore, more efficient therapies for B cell malignancies, in general, and Ph-like B-ALL specifically, that target, e.g., TSLPR cell surface antigen, including in combination with T cell activation (e.g., via CD3e), are needed.

### SUMMARY

The present disclosure provides antibodies and antigen-binding domain compositions capable of binding to thymic stromal lymphopoietin receptor (TSLPR). Also provided are methods of using such antibodies for the treatment of TSLPR-associated cancers (e.g., B-Cell Acute Lymphoblastic Leukemia (B-ALL)). In embodiments, the antibodies are heterodimeric antibodies that bind TSLPR and CD3 epsilon (i.e., anti-TSLPR x anti-CD3e) antibodies.

In one aspect provided herein is a bispecific antibody comprising: a) a thymic stromal lymphopoietin receptor (TSLPR) binding domain comprising i) a first variable heavy domain (VH1), and ii) a first variable light domain (VL1); and b) a CD3 epsilon (CD3e) binding domain comprising i) a second variable heavy domain (VH2), and ii) a second variable light domain (VL2).

In embodiments, the VH1 and VL1 are selected from one of the following:
(1) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(2) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:11 or a variant thereof; or
(3) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 12 or a variant thereof; or
(4) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:148 or a variant thereof; or
(5) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:149 or a variant thereof; or
(6) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:150 or a variant thereof; or
(7) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 151 or a variant thereof; or
(8) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 152 or a variant thereof; or
(9) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 153 or a variant thereof; or
(10) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154 or a variant thereof; or
(11) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 155 or a variant thereof; or
(12) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:156 or a variant thereof; or
(13) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(14) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 158 or a variant thereof; or
(15) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(16) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 160 or a variant thereof; or
(17) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 161 or a variant thereof; or
(18) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162 or a variant thereof; or
(19) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 163 or a variant thereof; or
(20) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 164 or a variant thereof; or
(21) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof.

In embodiments, the VH1 and VL1 are selected from the following:
(1) (i) a VH1 having an amino acid sequence of SEQ ID NO:7 or a variant thereof; and (ii) a VL1 having an amino acid sequence of SEQ ID NO:10 or a variant thereof; or
(2) (i) a VH1 having an amino acid sequence of SEQ ID NO:8 or a variant thereof; and (ii) a VL1 having an amino acid sequence of SEQ ID NO:11 or a variant thereof; or
(3) (i) a VH1 having an amino acid sequence of SEQ ID NO:9 or a variant thereof; and (ii) a VL1 having an amino acid sequence of SEQ ID NO:12 or a variant thereof; or
(4) (i) a VH1 having an amino acid sequence of SEQ ID NO:76 or a variant thereof; and (ii) a VL1 having an amino acid sequence of SEQ ID NO:148 or a variant thereof; or
(5) (i) a VH1 having an amino acid sequence of SEQ ID NO:77 or a variant thereof; and (ii) a VL1 having an amino acid sequence of SEQ ID NO:149 or a variant thereof; or
(6) (i) a VH1 having an amino acid sequence of SEQ ID NO:78 or a variant thereof; and (ii) a VL1 having an amino acid sequence of SEQ ID NO:150 or a variant thereof; or
(7) (i) a VH1 having an amino acid sequence of SEQ ID NO:79 or a variant thereof; and (ii) a VL1 having an amino acid sequence of SEQ ID NO:151 or a variant thereof; or
(8) (i) a VH1 having an amino acid sequence of SEQ ID NO:80 or a variant thereof; and (ii) a VL1 having an amino acid sequence of SEQ ID NO:152 or a variant thereof; or
(9) (i) a VH1 having an amino acid sequence of SEQ ID NO:81 or a variant thereof; and (ii) a VL1 having an amino acid sequence of SEQ ID NO:153 or a variant thereof; or
(10) (i) a VH1 having an amino acid sequence of SEQ ID NO:82 or a variant thereof; and (ii) a VL1 having an amino acid sequence of SEQ ID NO:154 or a variant thereof; or
(11) (i) a VH1 having an amino acid sequence of SEQ ID NO:83 or a variant thereof; and (ii) a VL1 having an amino acid sequence of SEQ ID NO:155 or a variant thereof; or
(12) (i) a VH1 having an amino acid sequence of SEQ ID NO:84 or a variant thereof; and (ii) a VL1 having an amino acid sequence of SEQ ID NO:156 or a variant thereof; or
(13) (i) a VH1 having an amino acid sequence of SEQ ID NO:85 or a variant thereof; and (ii) a VL1 having an amino acid sequence of SEQ ID NO: 157 or a variant thereof; or
(14) (i) a VH1 having an amino acid sequence of SEQ ID NO:86 or a variant thereof; and (ii) a VL1 having an amino acid sequence of SEQ ID NO: 158 or a variant thereof; or
(15) (i) a VH1 having an amino acid sequence of SEQ ID NO:87 or a variant thereof; and (ii) a VL1 having an amino acid sequence of SEQ ID NO:159 or a variant thereof; or
(16) (i) a VH1 having an amino acid sequence of SEQ ID NO:88 or a variant thereof; and (ii) a VL1 having an amino acid sequence of SEQ ID NO:160 or a variant thereof; or
(17) (i) a VH1 having an amino acid sequence of SEQ ID NO:89 or a variant thereof; and (ii) a VL1 having an amino acid sequence of SEQ ID NO:161 or a variant thereof; or
(18) (i) a VH1 having an amino acid sequence of SEQ ID NO:90 or a variant thereof; and (ii) a VL1 having an amino acid sequence of SEQ ID NO: 162 or a variant thereof, or
(19) (i) a VH1 having an amino acid sequence of SEQ ID NO:91 or a variant thereof; and (ii) a VL1 having an amino acid sequence of SEQ ID NO:163 or a variant thereof; or
(20) (i) a VH1 having an amino acid sequence of SEQ ID NO:92 or a variant thereof; and (ii) a VL1 having an amino acid sequence of SEQ ID NO: 164 or a variant thereof; or
(21) (i) a VH1 having an amino acid sequence of SEQ ID NO:93 or a variant thereof; and (ii) a VL1 having an amino acid sequence of SEQ ID NO:165 or a variant thereof.

In embodiments, the VH2 and VL2 are selected from the following:
(1) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(2) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(3) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(4) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(5) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(6) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

In embodiments, the VH2 and VL2 are selected from the following:
(1) (i) a VH2 having an amino acid sequence of SEQ ID NO:333 or a variant thereof; and (ii) a VL2 having an amino acid sequence of SEQ ID NO:337 or a variant thereof; or
(2) (i) a VH2 having an amino acid sequence of SEQ ID NO:344 or a variant thereof; and (ii) a VL2 having an amino acid sequence of SEQ ID NO:348 or a variant thereof; or
(3) (i) a VH2 having an amino acid sequence of SEQ ID NO:355 or a variant thereof; and (ii) a VL2 having an amino acid sequence of SEQ ID NO:359 or a variant thereof; or
(4) (i) a VH2 having an amino acid sequence of SEQ ID NO:366 or a variant thereof; and (ii) a VL2 having an amino acid sequence of SEQ ID NO:370 or a variant thereof; or
(5) (i) a VH2 having an amino acid sequence of SEQ ID NO:377 or a variant thereof; and (ii) a VL2 having an amino acid sequence of SEQ ID NO:381 or a variant thereof; or
(6) (i) a VH2 having an amino acid sequence of SEQ ID NO:388 or a variant thereof; and (ii) a VL2 having an amino acid sequence of SEQ ID NO:392 or a variant thereof.

In embodiments, the bispecific antibody further comprises a first Fc domain and a second Fc domain.

In embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W:T366S/L368A/Y407V, wherein numbering is according to EU numbering. In embodiments, the first and second Fc domains comprise heterodimerization skew variants S364K/E357Q: L368D/K370S, wherein numbering is according to EU numbering.

In embodiments, the first and second Fc domains each comprise one or more ablation variants. In some embodiments, the one or more ablation variants comprise E233P/L234V/L235A/G236del/S267K, wherein numbering is according to EU numbering.

In embodiments, the one of the first or second monomer further comprises one or more pI variants. In embodiments, the pI variants N208D/Q295E/N384D/Q418E/N421D, wherein numbering is according to EU numbering.

In one aspect, provided herein is a heterodimeric antibody that includes a first monomer, a second monomer, and a light chain. The first monomer comprises: i) a single chain variable fragment (scFv); and ii) a first Fc domain, wherein the scFv is covalently attached to the N-terminus of the first Fc domain using a domain linker. The second monomer comprises, from N-terminal to C-terminal, a VH1-CH1-hinge-CH2-CH3, wherein VH1 is a first variable heavy domain and CH2-CH3 is a second Fc domain. The light chain comprises, from N-terminal to C-terminal, VL1-CL, wherein VL1 is a first variable light domain and CL is a constant light domain. Further, the scFv comprises a second VH domain (VH2), a scFv linker, and a second variable light domain (VL2), the VH1 and the VL1 together form a first antigen binding domain (ABD) and the VH2 and the VL2 together form a second ABD, and one of the first ABD and second ABD is a thymic stromal lymphopoietin receptor (TSLPR) binding domain and the other of the first ABD and second ABD is a CD3 epsilon (CD3e) binding domain.

In embodiments, In the scFv comprises, from N-terminal to C-terminal, VH2-scFv linker-VL2. In embodiments, the scFv comprises, from N-terminal to C-terminal, VL2-scFv linker-VH2.

In embodiments, the first ABD is the TSLPR binding domain and the second ABD is the CD3e binding domain. In embodiments, the VH1 and VL1 are selected from one of the following:
(1) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(2) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:11 or a variant thereof; or
(3) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 12 or a variant thereof; or
(4) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:148 or a variant thereof; or
(5) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:149 or a variant thereof; or
(6) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 150 or a variant thereof; or
(7) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:151 or a variant thereof; or
(8) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:152 or a variant thereof; or
(9) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:153 or a variant thereof; or
(10) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154 or a variant thereof; or
(11) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 155 or a variant thereof; or
(12) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 156 or a variant thereof; or
(13) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(14) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 158 or a variant thereof, or
(15) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(16) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 160 or a variant thereof; or
(17) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 161 or a variant thereof; or
(18) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162 or a variant thereof; or
(19) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 163 or a variant thereof; or
(20) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 164 or a variant thereof; or
(21) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:165 or a variant thereof.

In embodiments, the VH1 and VL1 are selected from the following:
(1) (i) a VH1 having an amino acid sequence of SEQ ID NO:7 or a variant thereof; and (ii) a VL1 having an amino acid sequence of SEQ ID NO:10 or a variant thereof; or
(2) (i) a VH1 having an amino acid sequence of SEQ ID NO:8 or a variant thereof; and (ii) a VL1 having an amino acid sequence of SEQ ID NO:11 or a variant thereof; or
(3) (i) a VH1 having an amino acid sequence of SEQ ID NO:9 or a variant thereof; and (ii) a VL1 having an amino acid sequence of SEQ ID NO:12 or a variant thereof; or
(4) (i) a VH1 having an amino acid sequence of SEQ ID NO:76 or a variant thereof; and (ii) a VL1 having an amino acid sequence of SEQ ID NO:148 or a variant thereof; or
(5) (i) a VH1 having an amino acid sequence of SEQ ID NO:77 or a variant thereof; and (ii) a VL1 having an amino acid sequence of SEQ ID NO:149 or a variant thereof; or
(6) (i) a VH1 having an amino acid sequence of SEQ ID NO:78 or a variant thereof; and (ii) a VL1 having an amino acid sequence of SEQ ID NO: 150 or a variant thereof; or
(7) (i) a VH1 having an amino acid sequence of SEQ ID NO:79 or a variant thereof; and (ii) a VL1 having an amino acid sequence of SEQ ID NO:151 or a variant thereof; or
(8) (i) a VH1 having an amino acid sequence of SEQ ID NO:80 or a variant thereof; and (ii) a VL1 having an amino acid sequence of SEQ ID NO: 152 or a variant thereof; or
(9) (i) a VH1 having an amino acid sequence of SEQ ID NO:81 or a variant thereof; and (ii) a VL1 having an amino acid sequence of SEQ ID NO:153 or a variant thereof; or
(10) (i) a VH1 having an amino acid sequence of SEQ ID NO:82 or a variant thereof; and (ii) a VL1 having an amino acid sequence of SEQ ID NO:154 or a variant thereof; or
(11) (i) a VH1 having an amino acid sequence of SEQ ID NO:83 or a variant thereof; and (ii) a VL1 having an amino acid sequence of SEQ ID NO:155 or a variant thereof; or
(12) (i) a VH1 having an amino acid sequence of SEQ ID NO:84 or a variant thereof; and (ii) a VL1 having an amino acid sequence of SEQ ID NO:156 or a variant thereof; or
(13) (i) a VH1 having an amino acid sequence of SEQ ID NO:85 or a variant thereof; and (ii) a VL1 having an amino acid sequence of SEQ ID NO:157 or a variant thereof; or
(14) (i) a VH1 having an amino acid sequence of SEQ ID NO:86 or a variant thereof; and (ii) a VL1 having an amino acid sequence of SEQ ID NO:158 or a variant thereof; or
(15) (i) a VH1 having an amino acid sequence of SEQ ID NO:87 or a variant thereof; and (ii) a VL1 having an amino acid sequence of SEQ ID NO:159 or a variant thereof; or
(16) (i) a VH1 having an amino acid sequence of SEQ ID NO:88 or a variant thereof; and (ii) a VL1 having an amino acid sequence of SEQ ID NO:160 or a variant thereof; or
(17) (i) a VH1 having an amino acid sequence of SEQ ID NO:89 or a variant thereof; and (ii) a VL1 having an amino acid sequence of SEQ ID NO:161 or a variant thereof; or
(18) (i) a VH1 having an amino acid sequence of SEQ ID NO:90 or a variant thereof; and (ii) a VL1 having an amino acid sequence of SEQ ID NO:162 or a variant thereof; or
(19) (i) a VH1 having an amino acid sequence of SEQ ID NO:91 or a variant thereof; and (ii) a VL1 having an amino acid sequence of SEQ ID NO:163 or a variant thereof; or
(20) (i) a VH1 having an amino acid sequence of SEQ ID NO:92 or a variant thereof; and (ii) a VL1 having an amino acid sequence of SEQ ID NO:164 or a variant thereof; or
(21) (i) a VH1 having an amino acid sequence of SEQ ID NO:93 or a variant thereof; and (ii) a VL1 having an amino acid sequence of SEQ ID NO: 165 or a variant thereof.

In embodiments, the VH2 and VL2 are selected from one of the following:
(1) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(2) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(3) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(4) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(5) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(6) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

In embodiments, VH2 and VL2 are selected from one of the following:
(1) (i) a VH2 having an amino acid sequence of SEQ ID NO:333 or a variant thereof; and (ii) a VL2 having an amino acid sequence of SEQ ID NO:337 or a variant thereof; or
(2) (i) a VH2 having an amino acid sequence of SEQ ID NO:344 or a variant thereof; and (ii) a VL2 having an amino acid sequence of SEQ ID NO:348 or a variant thereof; or
(3) (i) a VH2 having an amino acid sequence of SEQ ID NO:355 or a variant thereof; and (ii) a VL2 having an amino acid sequence of SEQ ID NO:359 or a variant thereof; or
(4) (i) a VH2 having an amino acid sequence of SEQ ID NO:366 or a variant thereof; and (ii) a VL2 having an amino acid sequence of SEQ ID NO:370 or a variant thereof; or
(5) (i) a VH2 having an amino acid sequence of SEQ ID NO:377 or a variant thereof; and (ii) a VL2 having an amino acid sequence of SEQ ID NO:381 or a variant thereof; or
(6) (i) a VH2 having an amino acid sequence of SEQ ID NO:388 or a variant thereof; and (ii) a VL2 having an amino acid sequence of SEQ ID NO:392 or a variant thereof.

In embodiments, the bispecific antibody further comprises a first Fc domain and a second Fc domain.

In embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W:T366S/L368A/Y407V, wherein numbering is according to EU numbering. In embodiments, the first and second Fc domains comprise heterodimerization skew variants S364K/E357Q: L368D/K370S, wherein numbering is according to EU numbering.

In embodiments, the first and second Fc domains each comprise one or more ablation variants. In some embodiments, the one or more ablation variants comprise E233P/L234V/L235A/G236del/S267K, wherein numbering is according to EU numbering.

In embodiments, the one of the first or second monomer further comprises one or more pI variants. In embodiments, the pI variants N208D/Q295E/N384D/Q418E/N421D, wherein numbering is according to EU numbering.

In embodiments, the CH1-hinge-CH2-CH3 of the second monomer comprises amino acid variants E233P/L234V/L235A/G236del/S267K/L368D/K370S/N208D/Q295E/N384D/Q418E/N421 D, and the first Fc domain comprises amino acid variants E233P/L234V/L235A/G236del/S267K/S364K/E357Q, wherein numbering is according to EU numbering.

In embodiments, the first and second variant Fc domains each further comprise amino acid variants 428L/434S. In embodiments, the scFv linker is GKPGSGKPGSGKPGSGKPGS.

In one aspect, provided herein is a heterodimeric antibody that includes a first monomer, a second monomer, and a light chain. The first monomer comprises, from N-terminal to C-terminal, VH1-CH1-first domain linker-scFv-second domain linker-CH2-CH3, wherein VH1 is a first variable heavy domain, and CH2-CH3 is a first Fc domain. The second monomer comprises, from N-terminal to C-terminal, a VH1-CH1-hinge-CH2-CH3, wherein CH2-CH3 is a second Fc domain. The light chain comprises, from N-terminal to C-terminal, VL1-CL, wherein VL1 is a first variable light domain and CL is a constant light domain. Further, the scFv comprises a second VH domain (VH2), a scFv linker, and a second variable light domain (VL2), each of the VH1s and the VL1 together form a first antigen binding domain (ABD), and the VH2 and the VL2 form a second ABD, and one of the first ABDs and second ABD is a thymic stromal lymphopoietin receptor (TSLPR) binding domain and the other of the first ABDs and second ABD is a CD3 epsilon (CD3e) binding domain.

In embodiments, the scFv comprises, from N-terminal to C-terminal, VH2-scFv linker-VL2. In embodiments, the scFv comprises, from N-terminal to C-terminal, VL2-scFv linker-VH2.

In embodiments, the first ABDs are the TSLPR binding domains and the second ABD is the CD3e binding domain.

In embodiments, the VH1s and VL1s are selected from one of the following:
(1) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(2) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 11 or a variant thereof; or
(3) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 12 or a variant thereof; or
(4) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 148 or a variant thereof; or
(5) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:149 or a variant thereof; or
(6) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:150 or a variant thereof; or
(7) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 151 or a variant thereof; or
(8) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 152 or a variant thereof; or
(9) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 153 or a variant thereof; or
(10) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154 or a variant thereof; or
(11) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 155 or a variant thereof; or
(12) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 156 or a variant thereof; or
(13) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(14) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 158 or a variant thereof; or
(15) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(16) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 160 or a variant thereof; or
(17) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:161 or a variant thereof; or
(18) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162 or a variant thereof; or
(19) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 163 or a variant thereof; or
(20) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 164 or a variant thereof; or
(21) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof.

In embodiments, the VH1s and VL1s are selected from the following:
(1) (i) a VH1 having an amino acid sequence of SEQ ID NO:7 or a variant thereof; and (ii) a VL1 having an amino acid sequence of SEQ ID NO:10 or a variant thereof; or
(2) (i) a VH1 having an amino acid sequence of SEQ ID NO:8 or a variant thereof; and (ii) a VL1 having an amino acid sequence of SEQ ID NO:11 or a variant thereof; or
(3) (i) a VH1 having an amino acid sequence of SEQ ID NO:9 or a variant thereof; and (ii) a VL1 having an amino acid sequence of SEQ ID NO: 12 or a variant thereof; or
(4) (i) a VH1 having an amino acid sequence of SEQ ID NO:76 or a variant thereof; and (ii) a VL1 having an amino acid sequence of SEQ ID NO:148 or a variant thereof; or
(5) (i) a VH1 having an amino acid sequence of SEQ ID NO:77 or a variant thereof; and (ii) a VL1 having an amino acid sequence of SEQ ID NO: 149 or a variant thereof; or
(6) (i) a VH1 having an amino acid sequence of SEQ ID NO:78 or a variant thereof; and (ii) a VL1 having an amino acid sequence of SEQ ID NO:150 or a variant thereof; or
(7) (i) a VH1 having an amino acid sequence of SEQ ID NO:79 or a variant thereof; and (ii) a VL1 having an amino acid sequence of SEQ ID NO:151 or a variant thereof; or
(8) (i) a VH1 having an amino acid sequence of SEQ ID NO:80 or a variant thereof; and (ii) a VL1 having an amino acid sequence of SEQ ID NO: 152 or a variant thereof; or
(9) (i) a VH1 having an amino acid sequence of SEQ ID NO:81 or a variant thereof; and (ii) a VL1 having an amino acid sequence of SEQ ID NO:153 or a variant thereof; or
(10) (i) a VH1 having an amino acid sequence of SEQ ID NO:82 or a variant thereof; and (ii) a VL1 having an amino acid sequence of SEQ ID NO:154 or a variant thereof; or
(11) (i) a VH1 having an amino acid sequence of SEQ ID NO:83 or a variant thereof; and (ii) a VL1 having an amino acid sequence of SEQ ID NO: 155 or a variant thereof; or
(12) (i) a VH1 having an amino acid sequence of SEQ ID NO:84 or a variant thereof; and (ii) a VL1 having an amino acid sequence of SEQ ID NO:156 or a variant thereof; or
(13) (i) a VH1 having an amino acid sequence of SEQ ID NO:85 or a variant thereof; and (ii) a VL1 having an amino acid sequence of SEQ ID NO: 157 or a variant thereof; or
(14) (i) a VH1 having an amino acid sequence of SEQ ID NO:86 or a variant thereof; and (ii) a VL1 having an amino acid sequence of SEQ ID NO: 158 or a variant thereof; or
(15) (i) a VH1 having an amino acid sequence of SEQ ID NO:87 or a variant thereof; and (ii) a VL1 having an amino acid sequence of SEQ ID NO:159 or a variant thereof; or
(16) (i) a VH1 having an amino acid sequence of SEQ ID NO:88 or a variant thereof; and (ii) a VL1 having an amino acid sequence of SEQ ID NO: 160 or a variant thereof; or
(17) (i) a VH1 having an amino acid sequence of SEQ ID NO:89 or a variant thereof; and (ii) a VL1 having an amino acid sequence of SEQ ID NO: 161 or a variant thereof; or
(18) (i) a VH1 having an amino acid sequence of SEQ ID NO:90 or a variant thereof; and (ii) a VL1 having an amino acid sequence of SEQ ID NO:162 or a variant thereof; or
(19) (i) a VH1 having an amino acid sequence of SEQ ID NO:91 or a variant thereof; and (ii) a VL1 having an amino acid sequence of SEQ ID NO: 163 or a variant thereof; or
(20) (i) a VH1 having an amino acid sequence of SEQ ID NO:92 or a variant thereof; and (ii) a VL1 having an amino acid sequence of SEQ ID NO:164 or a variant thereof; or
(21) (i) a VH1 having an amino acid sequence of SEQ ID NO:93 or a variant thereof; and (ii) a VL1 having an amino acid sequence of SEQ ID NO:165 or a variant thereof.

In embodiments, the VH2 and VL2 are selected from one of the following:
(1) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(2) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(3) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(4) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(5) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(6) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

In embodiments, the VH2 and VL2 are selected from one of the following:
(1) (i) a VH2 having an amino acid sequence of SEQ ID NO:333 or a variant thereof; and (ii) a VL2 having an amino acid sequence of SEQ ID NO:337 or a variant thereof; or
(2) (i) a VH2 having an amino acid sequence of SEQ ID NO:344 or a variant thereof; and (ii) a VL2 having an amino acid sequence of SEQ ID NO:348 or a variant thereof; or
(3) (i) a VH2 having an amino acid sequence of SEQ ID NO:355 or a variant thereof; and (ii) a VL2 having an amino acid sequence of SEQ ID NO:359 or a variant thereof; or
(4) (i) a VH2 having an amino acid sequence of SEQ ID NO:366 or a variant thereof; and (ii) a VL2 having an amino acid sequence of SEQ ID NO:370 or a variant thereof; or
(5) (i) a VH2 having an amino acid sequence of SEQ ID NO:377 or a variant thereof; and (ii) a VL2 having an amino acid sequence of SEQ ID NO:381 or a variant thereof; or
(6) (i) a VH2 having an amino acid sequence of SEQ ID NO:388 or a variant thereof; and (ii) a VL2 having an amino acid sequence of SEQ ID NO:392 or a variant thereof.

In embodiments, the bispecific antibody further comprises a first Fc domain and a second Fc domain.

In embodiments, the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W:T366S/L368A/Y407V, wherein numbering is according to EU numbering. In embodiments, the first and second Fc domains comprise heterodimerization skew variants S364K/E357Q: L368D/K370S, wherein numbering is according to EU numbering.

In embodiments, the first and second Fc domains each comprise one or more ablation variants. In some embodiments, the one or more ablation variants comprise E233P/L234V/L235A/G236del/S267K, wherein numbering is according to EU numbering.

In embodiments, the one of the first or second monomer further comprises one or more pI variants. In embodiments, the pI variants N208D/Q295E/N384D/Q418E/N421D, wherein numbering is according to EU numbering.

In embodiments, the CH1-hinge-CH2-CH3 of the second monomer comprises amino acid variants E233P/L234V/L235A/G236del/S267K/L368D/K370S/N208D/Q295E/N384D/Q418E/N421 D, and the first Fc domain comprises amino acid variants E233P/L234V/L235A/G236del/S267K/S364K/E357Q, wherein numbering is according to EU numbering.

In embodiments, the first and second variant Fc domains each further comprise amino acid variants 428L/434S. In embodiments, the scFv linker is GKPGSGKPGSGKPGSGKPGS.

Also provided herein are nucleic acid compositions comprising nucleic acids encoding the antibodies described herein, expression vector compositions that include such nucleic acids, host cells for making the antibodies that comprise the expression vector compositions, and methods of making the antibodies.

In another aspect, provided herein is a method of treating a TSLPR-associated in a patient in need thereof, comprising administering to the patient an anti-TSLPR x anti-CD3e antibody described herein. In embodiments, the TSLPR-associated cancer is a leukemia one of the following leukemias: an Acute Lymphoblastic Leukemia, a B-cell Acute Lymphoblastic Leukemia, or a Philadelphia chromosome-like B Cell Acute Lymphoblastic Leukemia.

In some aspects, the antibody or antigen-binding fragment thereof capable of binding to TSLPR, comprises a heavy chain variable region that comprises an amino acid sequence selected from the group consisting of SEQ ID NO: 13, 40, 41, 16, 64, 65, 19, 52, 53, and combinations thereof; an amino acid sequence selected from the group consisting of SEQ ID NO: 14, 42, 43, 17, 66, 67, 20, 54, 55, and combinations thereof; and an amino acid sequence selected from the group consisting of SEQ ID NO: 15, 44, 45, 18, 68, 69, 21, 56, 57, and combinations thereof; and a light chain variable region that comprises an amino acid sequence selected from the group consisting of SEQ ID NO: 22, 46, 47, 25, 70, 71, 28, 58, 59 and combinations thereof; an amino acid sequence selected from the group consisting of SEQ ID NO: 23, 48, 49, 26, 72, 73, 29, 60, 61 and combinations thereof; and an amino acid sequence selected from the group consisting of SEQ ID NO: 24, 50, 51, 27, 74, 75, 30, 62, 63 and combinations thereof.

In some aspects, the antibody or antigen-binding fragment thereof comprises
a heavy chain variable region that comprises an amino acid sequence having SEQ ID NO: 13, 14, and 15, and a light chain variable region that comprises an amino acid sequence having SEQ ID NO: 22, 23, and 24.

In some aspects, the antibody or antigen-binding fragment thereof comprises a heavy chain variable region that comprises an amino acid sequence having SEQ ID NO: 40, 42, and 44, and a light chain variable region that comprises an amino acid sequence having SEQ ID NO: 46, 48, and 50.

In some aspects, the antibody or antigen-binding fragment thereof comprises a heavy chain variable region that comprises an amino acid sequence having SEQ ID NO: 41, 43, and 45, and the light chain variable region comprises an amino acid sequence having SEQ ID NO: 47, 49, and 51.

In some aspects, the antibody or antigen-binding fragment thereof comprises a heavy chain variable region that comprises an amino acid sequence having SEQ ID NO: 16, 17, and 18, and the light chain variable region comprises an amino acid sequence having SEQ ID NO: 25, 26, and 27.

In some aspects, the antibody or antigen-binding fragment thereof comprises a heavy chain variable region that comprises an amino acid sequence having SEQ ID NO: 64, 66, and 68, and the light chain variable region comprises an amino acid sequence having SEQ ID NO: 70, 72, and 74.

In some aspects, the antibody or antigen-binding fragment thereof comprises a heavy chain variable region that comprises an amino acid sequence having SEQ ID NO: 65, 67, and 69, and the light chain variable region comprises an amino acid sequence having SEQ ID NO: 71, 73, and 75.

In some aspects, the antibody or antigen-binding fragment thereof comprises a heavy chain variable region that comprises an amino acid sequence having SEQ ID NO: 19, 20, and 21, and the light chain variable region comprises an amino acid sequence having SEQ ID NO: 28, 29, and 30.

In some aspects, the antibody or antigen-binding fragment thereof comprises a heavy chain variable region that comprises an amino acid sequence having SEQ ID NO: 52, 54, and 56, and the light chain variable region comprises an amino acid sequence having SEQ ID NO: 58, 60, and 62.

In some aspects, the antibody or antigen-binding fragment thereof comprises a heavy chain variable region that comprises an amino acid sequence having SEQ ID NO: 53, 55, and 57, and the light chain variable region comprises an amino acid sequence having SEQ ID NO: 59, 61, and 63.

In some aspects, the antibody or antigen-binding fragment thereof comprises a heavy chain variable region (VH) and a light chain variable region (VL), wherein the VH comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 7 and the VL comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 10.

In some aspects, the antibody or antigen-binding fragment thereof comprises a heavy chain variable region (VH) and a light chain variable region (VL), wherein the VH comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 8 and the VL comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 11.

In some aspects, the antibody or antigen-binding fragment thereof comprises a heavy chain variable region (VH) and a light chain variable region (VL), wherein the VH comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 9 and the VL comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 12.

In some aspects, the antibody or antigen-binding fragment thereof capable of binding to TSLPR comprises a CDR H1, CDR H2, and CDR H3 comprising the CDR H1, CDR H2, and CDR H3 amino acid sequences of any one of SEQ ID NO: 7, SEQ ID NO: 8, or SEQ ID NO: 9 and a CDR L1, CDR L2, and CDR L3 comprising the CDR L1, CDR L2, and CDR L3 amino acid sequences of any one of SEQ ID NO: 10, SEQ ID NO: 11, or SEQ ID NO: 12.

In some aspects, the antibody or antigen-binding fragment thereof comprises CDRs that are Kabat-defined CDRs, AbM-defined CDRs, or IMGT-defined CDRs.

In some aspects, the antibody or antigen-binding fragment thereof comprises a heavy chain, wherein the heavy chain variable region comprises an amino acid sequence that is at least 80% identical to SEQ ID NO: 7, SEQ ID NO: 8, or SEQ ID NO: 9.

In some aspects, the antibody or antigen-binding fragment thereof comprises a heavy chain, wherein the heavy chain variable region comprises an amino acid sequence that is at least 85% identical to SEQ ID NO: 7, SEQ ID NO: 8, or SEQ ID NO: 9.

In some aspects, the antibody or antigen-binding fragment thereof comprises a heavy chain, wherein the heavy chain variable region comprises an amino acid sequence that is at least 90% identical to SEQ ID NO: 7, SEQ ID NO: 8, or SEQ ID NO: 9.

In some aspects, the antibody or antigen-binding fragment thereof comprises a heavy chain, wherein the heavy chain variable region comprises an amino acid sequence that is at least 95% identical to SEQ ID NO: 7, SEQ ID NO: 8, or SEQ ID NO: 9.

In some aspects, the antibody or antigen-binding fragment thereof comprises a heavy chain, wherein the heavy chain variable region comprises an amino acid sequence that is at least 98% identical to SEQ ID NO: 7, SEQ ID NO: 8, or SEQ ID NO: 9.

In some aspects, the antibody or antigen-binding fragment thereof comprises a heavy chain, wherein the heavy chain variable region comprises an amino acid sequence that is at least 99% identical to SEQ ID NO: 7, SEQ ID NO: 8, or SEQ ID NO: 9.

In some aspects, the antibody or antigen-binding fragment thereof comprises a heavy chain, wherein the heavy chain variable region comprises an amino acid sequence having SEQ ID NO: 7, SEQ ID NO: 8, or SEQ ID NO: 9.

In some aspects, the antibody or antigen-binding fragment thereof comprises a light chain, wherein the light chain variable region comprises an amino acid sequence that is at least 80% identical to SEQ ID NO: 10, SEQ ID NO: 11, or SEQ ID NO: 12.

In some aspects, the antibody or antigen-binding fragment thereof comprises a light chain, wherein the light chain variable region comprises an amino acid sequence that is at least 85% identical to SEQ ID NO: 10, SEQ ID NO: 11, or SEQ ID NO: 12.

In some aspects, the antibody or antigen-binding fragment thereof comprises a light chain, wherein the light chain variable region comprises an amino acid sequence that is at least 90% identical to SEQ ID NO: 10, SEQ ID NO: 11, or SEQ ID NO: 12.

In some aspects, the antibody or antigen-binding fragment thereof comprises a light chain, wherein the light chain variable region comprises an amino acid sequence that is at least 95% identical to SEQ ID NO: 10, SEQ ID NO: 11, or SEQ ID NO: 12.

In some aspects, the antibody or antigen-binding fragment thereof comprises a light chain, wherein the light chain variable region comprises an amino acid sequence that is at least 98% identical to SEQ ID NO: 10, SEQ ID NO: 11, or SEQ ID NO: 12.

In some aspects, the antibody or antigen-binding fragment thereof comprises a light chain, wherein the light chain variable region comprises an amino acid sequence that is at least 99% identical to SEQ ID NO: 10, SEQ ID NO: 11, or SEQ ID NO: 12.

In some aspects, the antibody or antigen-binding fragment thereof comprises a light chain, wherein the light chain variable region comprises an amino acid sequence having SEQ ID NO: 10, SEQ ID NO: 11, or SEQ ID NO: 12.

In some aspects, the heavy chain variable region and the light chain variable region of the antibody or antigen-binding fragment thereof form an antigen-binding site for a thymic stromal lymphopoietin receptor.

In some aspects, the antibody or antigen-binding fragment thereof is human, humanized, or chimeric.

In some aspects, the antibody or antigen-binding fragment thereof is an IgG antibody,

In some aspects, the antibody or antigen-binding fragment thereof is an IgG1 antibody or an IgG4 antibody.

In some aspects, the antibody or antigen-binding fragment thereof is an antigen-binding fragment of an antibody.

In some aspects, the antigen-binding fragment thereof is selected from the group consisting of Fab, F(ab′) 2, Fv, scFv, scFv-Fc, dsFv and a single domain molecule. In some aspects, the antigen-binding fragment is a scFv.

In some aspects, the antigen-binding fragment is a Fab.

In some aspects, the antigen-binding fragment is an intrabody.

In some aspects, the antigen-binding fragment is devoid of an Fc region.

In some aspects, the antibody or antigen-binding fragment thereof comprises a VH and a VL on the same polypeptide chain.

In some aspects, the VH and VL are connected by a linker.

In some aspects, the antibody or antigen-binding fragment thereof is conjugated to an agent selected from the group consisting of a therapeutic agent, a prodrug, a peptide, a protein, an enzyme, a virus, a lipid, a biological response modifier, a pharmaceutical agent, and PEG.

The present disclosure also provides bispecific binding proteins comprising three chains: a first chain comprising, in order from the N-terminus to the C-terminus, a heavy chain variable region, and a heavy chain constant region; a second chain comprising, in order from the N-terminus to the C-terminus, a light chain variable region, and a light chain constant region; and a third chain comprising, in order from the N-terminus to the C-terminus, a single chain variable fragment (scFv), and a heavy chain constant region; wherein the scFv of the third chain, in order from the N-terminus to the C-terminus, comprises a heavy chain variable region and a light chain variable region or, in order from the N-terminus to the C-terminus, comprises a light chain variable region and a heavy chain variable region; wherein the heavy chain variable region of the third chain comprises an amino acid sequence that comprises SEQ ID NO: 1, 2, and 3 and the light chain variable region of the third chain comprises an amino acid sequence that comprises SEQ ID NO; 4, 5, and 6; wherein the heavy chain variable region of the first chain and the heavy chain variable region of the third chain comprise different amino acid sequences and the light chain variable region of the second chain and the light chain variable region of the scFv of the third chain comprise different amino acid sequences; wherein the heavy chain variable region of the first chain comprises an amino acid sequence selected from the group consisting of SEQ ID NO: 13, 40, 41, 16, 64, 65, 19, 52, 53, and combinations thereof; an amino acid sequence selected from the group consisting of SEQ ID NO: 14, 42, 43, 17, 66, 67, 20, 54, 55, and combinations thereof; and an amino acid sequence selected from the group consisting of SEQ ID NO: 15, 44, 45, 18, 68, 69, 21, 56, 57, and combinations thereof; wherein the light chain variable region of the second chain comprises an amino acid sequence selected from the group consisting of SEQ ID NO: 22, 46, 47, 25, 70, 71, 28, 58, 59, and combinations thereof; an amino acid sequence selected from the group consisting of SEQ ID NO: 23, 48, 49, 26, 72, 73, 29, 60, 61, and combinations thereof; and an amino acid sequence selected from the group consisting of SEQ ID NO: 24, 50, 51, 27, 74, 75, 30, 62, 63, and combinations thereof; and wherein the heavy chain constant region of the first chain comprises an amino acid sequence that is at least 90% identical to the heavy chain constant region of the third chain and wherein the heavy chain constant region of the first chain and the heavy chain constant region of the third chain dimerize.

In some aspects of the bispecific binding proteins, a) the heavy chain variable region of the first chain comprises an amino acid sequence having SEQ ID NO: 13, 14, and 15, and the light chain variable region of the second chain comprises an amino acid sequence having SEQ ID NO: 22, 23, and 24; b) the heavy chain variable region of the first chain comprises an amino acid sequence having SEQ ID NO: 40, 42, and 44, and the light chain variable region of the second chain comprises an amino acid sequence having SEQ ID NO: 46, 48, and 50; c) the heavy chain variable region of the first chain comprises an amino acid sequence having SEQ ID NO: 41, 43, and 45, and the light chain variable region of the second chain comprises an amino acid sequence having SEQ ID NO: 47, 49, and 51; d) the heavy chain variable region of the first chain comprises an amino acid sequence having SEQ ID NO: 16, 17, and 18, and the light chain variable region of the second chain comprises an amino acid sequence having SEQ ID NO: 25, 26, and 27; e) the heavy chain variable region of the first chain comprises an amino acid sequence having SEQ ID NO: 64, 66, and 68, and the light chain variable region of the second chain comprises an amino acid sequence having SEQ ID NO: 70, 72, and 74; f) the heavy chain variable region of the first chain comprises an amino acid sequence having SEQ ID NO: 65, 67, and 69, and the light chain variable region of the second chain comprises an amino acid sequence having SEQ ID NO: 71, 73, and 75; g) the heavy chain variable region of the first chain comprises an amino acid sequence having SEQ ID NO: 19, 20, and 21, and the light chain variable region of the second chain comprises an amino acid sequence having SEQ ID NO: 28, 29, and 30; h) the heavy chain variable region of the first chain comprises an amino acid sequence having SEQ ID NO: 52, 54, and 56, and the light chain variable region of the second chain comprises an amino acid sequence having SEQ ID NO: 58, 60, and 62; or i) the heavy chain variable region of the first chain comprises an amino acid sequence having SEQ ID NO: 53, 55, and 57, and the light chain variable region of the second chain comprises an amino acid sequence having SEQ ID NO: 59, 61, and 63.

In some aspects, the bispecific binding proteins comprise a heavy chain constant region of the first chain which comprises a CH1 domain and a Fc region comprising CH2 and CH3 domains and a heavy chain constant region of the third chain which comprises a CH1 domain and a Fc region comprising CH2 and CH3 domains.

In some aspects, the heavy chain constant region of the first chain of the bispecific binding protein comprises an amino acid sequence that is at least 95% identical to the heavy chain constant region of the third chain.

In some aspects, the heavy chain constant region of the first chain of the bispecific binding protein comprises an amino acid sequence that is at least 97% identical to the heavy chain constant region of the third chain.

In some aspects, the heavy chain constant region of the first chain and the heavy chain constant region of the third chain are subject to at least one modification to reduce the formation of a homodimer.

In some aspects, the at least one modification comprises substituting an amino acid residue at the interface of the Fc region of the first chain with an amino acid residue having a side chain with an opposite charge to a residue at the interface of the Fc region of the third chain.

In some aspects, the amino acid at the interface of the Fc region of the first chain of the bispecific binding protein is selected from a glutamic acid, a serine, and an aspartic acid.

In some aspects, the amino acid at the interface of the Fc region of the third chain of the bispecific binding protein is selected from a glutamine, a lysine, a leucine, and an asparagine.

In some aspects, the heavy chain variable region of the first chain of the bispecific binding protein comprises an amino acid sequence that is at least 80% identical to SEQ ID NO: 7, SEQ ID NO: 8, or SEQ ID NO: 9.

In some aspects, the heavy chain variable region of the first chain of the bispecific binding protein comprises an amino acid sequence that is at least 85% identical to SEQ ID NO: 7, SEQ ID NO: 8, or SEQ ID NO: 9.

In some aspects, the heavy chain variable region of the first chain of the bispecific binding protein comprises an amino acid sequence that is at least 90% identical to SEQ ID NO: 7, SEQ ID NO: 8, or SEQ ID NO: 9.

In some aspects, the heavy chain variable region of the first chain of the bispecific binding protein comprises an amino acid sequence that is at least 95% identical to SEQ ID NO: 7, SEQ ID NO: 8, or SEQ ID NO: 9.

In some aspects, the heavy chain variable region of the first chain of the bispecific binding protein comprises an amino acid sequence having SEQ ID NO: 7, SEQ ID NO: 8, or SEQ ID NO: 9.

In some aspects, the light chain variable region of the second chain of the bispecific binding protein comprises an amino acid sequence that is at least 80% identical to SEQ ID NO: 10, SEQ ID NO: 11, or SEQ ID NO: 12.

In some aspects, the light chain variable region of the second chain of the bispecific binding protein comprises an amino acid sequence that is at least 85% identical to SEQ ID NO: 10, SEQ ID NO: 11, or SEQ ID NO: 12.

In some aspects, the light chain variable region of the second chain of the bispecific binding protein comprises an amino acid sequence that is at least 90% identical to SEQ ID NO: 10, SEQ ID NO: 11, or SEQ ID NO: 12.

In some aspects, the light chain variable region of the second chain of the bispecific binding protein comprises an amino acid sequence that is at least 95% identical to SEQ ID NO: 10, SEQ ID NO: 11, or SEQ ID NO: 12.

In some aspects, the light chain variable region of the second chain of the bispecific binding protein comprises an amino acid sequence having SEQ ID NO: 10, SEQ ID NO: 11, or SEQ ID NO: 12.

In some aspects, the heavy chain variable region of the first chain and the light chain variable region of the second chain of the bispecific binding protein form an antigen-binding site for a thymic stromal lymphopoietin receptor.

In some aspects, the heavy chain variable region and the light chain variable region of the third chain form an antigen-binding site for CD3.

In some aspects, the heavy chain constant region of the first chain and the heavy chain constant region of the third chain of the bispecific binding protein further comprise a hinge region between the CH1 domain and the Fc region.

In some aspects, an intermolecular disulfide bond is provided between the hinge region of the first chain and the hinge region of the third chain by introducing cysteine at corresponding positions between the CH3 domain of the Fc region of the first chain and the CH3 domain of the Fc region of the third chain.

The present disclosure provides pharmaceutical compositions comprising the bispecific binding proteins of the invention, and at least one pharmaceutically acceptable excipient.

The present disclosure provides formulations comprising the bispecific binding proteins of the invention.

The present disclosure provides methods of treating a cancer in a subject in need thereof, the method comprising administering to the subject an effective amount of a pharmaceutical composition or a formulation comprising a bispecific binding protein according to the invention.

In some aspects, the cancer is Acute Lymphoblastic Leukemia, B-cell Acute Lymphoblastic Leukemia, and specifically Philadelphia chromosome-like B Cell Acute Lymphoblastic Leukemia. In some aspects, the cancer overexpresses TSLPR.

In some aspects, the methods further comprise administering to the subject an effective amount of an additional cancer therapy. In some aspects, the additional cancer therapy is selected from radiation therapy, surgery, chemotherapy, gene therapy, DNA therapy, viral therapy, RNA therapy, immunotherapy, bone marrow transplantation, nanotherapy, monoclonal antibody therapy, and a combination thereof.

In some aspects, the methods further comprise performing a chemiluminescence immunoassay (CLIA) on cells of the subject to quantify TSLPR expression; performing a genetic analysis including genome and/or transcriptome profiling to quantify genes and/or transcripts of Ph-like B-ALL; or both. The present disclosure provides a kit comprising a bispecific binding protein as described herein, a pharmaceutical composition as described herein, or a formulation as described herein.

### BRIEF DESCRIPTION OF THE DRAWINGS

FIG. 1A shows a histogram of TSLPR expression in pre-B-ALL cells of a first patient sample (Pt4). FIG. 1B shows a histogram of TSLPR expression in pre-B-ALL cells of a second patient sample (Pt8). FIG. 1C shows a histogram of TSLPR expression in pre-B-ALL cells of a third patient sample (Pt13). FIG. 1D shows a histogram of TSLPR expression in pre-B-ALL cells of a fourth patient sample (Pt14). FIG. 1E shows a histogram of TSLPR expression in pre-B-ALL cells of a fifth patient sample (Pt15). FIG. 1F shows a histogram of CD19 expression in pre-B-ALL cells of the first patient sample (Pt4). FIG. 1G shows a histogram of CD19 expression in pre-B-ALL cells of the second patient sample (Pt8). FIG. 1H shows a histogram of CD19 expression in pre-B-ALL cells of the third patient sample (Pt13). FIG. 1I shows a histogram of CD19 expression in pre-B-ALL cells of the fourth patient sample (Pt14). FIG. 1J shows a histogram of CD19 expression in pre-B-ALL cells of the fifth patient sample (Pt15).

FIG. 2A shows a sensogram of binding of a TSLPR x CD3 bispecific antibody to TSLPR. FIG. 2B shows a sensogram of binding of a TSLPR x CD3 bispecific antibody to CD3-Fc. FIG. 2C shows an Ultra Performance Liquid Chromatography (UPLC™) of the TSLPR x CD3 bispecific antibody. FIG. 2D shows a mass spectrum identifying the whole bispecific TSLPR x CD3 molecule. FIG. 2E shows a cytotoxicity assay and half-maximal effective concentrations (EC₅₀) for a monospecific TSLPR antibody and the bispecific TSLPR x CD3 antibody.

FIG. 3 shows bioluminescence imaging results over time of mice xenografted with luciferase-expressing ALL blasts and donor-derived PBMCs from two different human donors (PBMC-296, left panels, and PBMC-879, right panels) and injected with TSLPR x CD3e bispecific antibodies or PBS (control).

FIG. 4A shows the percentage of live patient-derived ALL blasts in the blood of mice xenografted with the patient-derived blasts and injected with PBMC-875 cells and different concentrations of TSLPR x CD3 bispecific antibody; control mice were injected with PBS only or PBMC-875 cells and PBS. FIG. 4B shows the percentage of live patient-derived ALL blasts in the bone marrow of mice xenografted with the patient-derived ALL blasts and injected with PBMC-875 cells and different concentrations of TSLPR x CD3 bispecific antibody; control mice were injected with PBS only or PBMC-875 cells and PBS. FIG. 4C shows the percentage of live patient-derived ALL blasts in the spleen of mice xenografted with the patient-derived ALL blasts and injected with PBMC-875 cells and different concentrations of TSLPR x CD3 bispecific antibody; control mice were injected with PBS only or PBMC-875 cells and PBS.

FIG. 5A shows the percentage of CD3⁺ cells in blood over time in mice xenografted with patient-derived ALL blasts and injected with PBMC-875 cells and 1 mg/kg TSLPR x CD3 bispecific antibody. FIG. 5B shows the percentage of CD3⁺ cells in the bone marrow of mice xenografted with patient-derived ALL blasts and injected with PBMC-875 cells and 0.01, 0.1, and 1 mg/kg TSLPR x CD3 bispecific antibody. FIG. 5C shows the percentage of CD3⁺ cells in the spleen of mice xenografted with patient-derived ALL blasts and injected with PBMC-875 cells and 0.01, 0.1, and 1 mg/kg of TSLPR x CD3 bispecific antibody.

FIG. 6 shows the serum concentrations of TSLPR x CD3 bispecific antibody in non-human primates 4 and 24 h after a first and a second dose of TSLPR x CD3 antibody.

FIG. 7A shows the fold changes in serum levels of IFN-γ in the serum of non-human primates 4 and 24 h after injection of different concentrations of the TSLPR x CD3 bispecific antibody. FIG. 7B shows the fold changes in serum levels of IL-6 in the serum of non-human primates 4 and 24 h after injection of different concentrations of the TSLPR x CD3 bispecific antibody. FIG. 7C shows the fold changes in serum levels of IL-8 in the serum of non-human primates 4 and 24 h after injection of different concentrations of the TSLPR x CD3 bispecific antibody. FIG. 7D shows the fold changes in serum levels of IL-10 in the serum of non-human primates 4 and 24 h after injection of different concentrations of the TSLPR x CD3 bispecific antibody. FIG. 7E shows the fold changes in serum levels of MCP-1 in the serum of non-human primates 4 and 24 h after injection of different concentrations of the TSLPR x CD3 bispecific antibody. FIG. 7F shows the fold changes in serum levels of TNF-α in the serum of non-human primates 4 and 24 h after injection of different concentrations of the TSLPR x CD3 bispecific antibody.

FIG. 8 shows the pharmacokinetics of TSLPR x CD3 bispecific antibody in mice as serum concentrations over time of TSLPR x CD3 bispecific antibody injected at doses of 0.3 and 1 mg/kg.

FIG. 9A shows relative light units (RLU) for fluorescent signals of an ADCC assay performed with target cells in the presence of different concentrations of TSLPR-004 and TSLPR-006 antibodies. FIG. 9B shows percent killing of TSLPR-expressing REH tumor target cells in a primary ADCC assay in the presence of different concentrations of TSLPR-004 and TSLPR-006 antibodies. FIG. 9C shows percent phagocytosis of TSLPR-expressing REH cells by human PBMC-derived monocytes in the presence of 1 μg/ml and 10 μg/ml TSLPR-006 antibodies or in the presence of 10 μg/ml huIgG1 control antibodies, no antibodies or REH cells alone. The top row shows phagocytosis results at effector:target ratios of 1:1 and the bottom row shows phagocytosis results at effector:target ratios of 2:1. FIG. 9D shows western blots of total and phosphorylated Jak2 and Stat5 in TSLPR-antibody-coated REH cells after stimulation with different concentrations of TSLP. FIG. 9E shows western blots of total and phosphorylated S6R in TSLPR-antibody-coated REH cells prior to and after stimulation with different concentrations of TSLP (right panel) and REH cells incubated with Herceptin and 2D10 control antibodies prior to and after stimulation with different concentrations of TSLP (left panel).

FIG. 10A shows a sensogram of binding of TSLPR antibody clone 2E4 to TSLPR. FIG. 10B shows a sensogram of binding of TSLPR antibody clone 2H8 to TSLPR. FIG. 10C shows a sensogram of binding of TSLPR antibody clone 3E5 to TSLPR. FIG. 10D shows a sensogram of binding of TSLPR antibody clone 1A9 to TSLPR. FIG. 10E shows a sensogram of binding of TSLPR antibody clone 1A10 to TSLPR. FIG. 10F shows a sensogram of binding of TSLPR antibody clone 1B7 to TSLPR. FIG. 10G shows a sensogram of binding of TSLPR antibody clone 1B9 to TSLPR. FIG. 10H shows a sensogram of binding of TSLPR antibody clone 1D10 to TSLPR. FIG. 10I shows a sensogram of binding of TSLPR antibody clone 1F6 to TSLPR. FIG. 10J shows a sensogram of binding of TSLPR antibody clone 1G3 to TSLPR. FIG. 10K shows a sensogram of binding of TSLPR antibody clone 3D8 to TSLPR. FIG. 10L shows a sensogram of binding of TSLPR antibody clone 10C4 to TSLPR. FIG. 10M shows a sensogram of binding of TSLPR antibody clone 2A4 to TSLPR. FIG. 10N shows a sensogram of binding of TSLPR antibody clone 2B2 to TSLPR. FIG. 10O shows a sensogram of binding of TSLPR antibody clone 2D11 to TSLPR. FIG. 10P shows a sensogram of binding of TSLPR antibody clone 2E6 to TSLPR. FIG. 10Q shows a sensogram of binding of TSLPR antibody clone 2F1 to TSLPR. FIG. 10R shows a sensogram of binding of TSLPR antibody clone 2F5 to TSLPR. FIG. 10S shows a sensogram of binding of TSLPR antibody clone 2H1 to TSLPR. FIG. 10T shows a sensogram of binding of TSLPR antibody clone 2H4 to TSLPR. FIG. 10U shows a sensogram of binding of TSLPR antibody clone 1C3 to TSLPR.

FIG. 11A shows a graph of tumor volume over time in tumor-bearing mice treated with TSLPR antibodies 1A9, 1B7, 1C3, and Fc-modified 1C3 compared to Herceptin control. FIG. 11B shows the percentages of tumor growth inhibition of TSLPR antibodies 1A9, 1B7, 1C3, and Fc-modified 1C3.

FIGS. 12A-12F depict useful pairs of heterodimerization variant sets (including skew and pI variants). In FIG. 12F, there are variants for which there are no corresponding “monomer 2” variants. Such variants are pI variants that can be used alone on either monomer of a bispecific antibody, or included, for example, on the non-scFv side of a format that utilizes an scFv as a component and an appropriate charged scFv linker can be used on the second monomer that utilizes an scFv as the CD3 binding domain. Suitable charged linkers are shown in FIG. 15.

FIG. 13 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 14 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 15 depicts a number of charged scFv linkers that find use in increasing or decreasing the pI of the subject heterodimeric bispecific antibodies that utilize one or more scFv as a component, as described herein (e.g., anti-TSLPR x anti-CD3e bsAbs). The (+H) positive linker finds particular use herein, particularly with anti-CD3 VL and VH sequences shown herein. A single prior art scFv linker with a single charge is referenced as “Whitlow,” from Whitlow et al., Protein Engineering 6 (8): 989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIGS. 16A and 16B depict a number of exemplary domain linkers. In some embodiments, these linkers find use linking a single-chain Fv to an Fc chain. In some embodiments, these linkers may be combined in any orientation. For example, a GGGGS linker may be combined with a “lower half hinge” linker at the N-terminus or at the C-terminus.

FIG. 17 shows a particularly useful bispecific antibody platform for the TSLPR x CD3 bsAbs of the invention. Although the platforms are described here in the context of the 1+1 Fab-scFv-Fc format, they can be adapted for use in other bispecific antibody formats.

FIG. 18 depicts various heterodimeric skew variant amino acid substitutions that can be used with the heterodimeric antibodies described herein.

FIGS. 19A-19E show the sequences of several useful heterodimeric TSLPR x CD3 bsAb backbones based on human IgG1, without the variable domain. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants on both chains. Heterodimeric Fc backbone 14 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants and M252Y/S254T/T256E half-life extension variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 16 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 17 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (G446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 20 depicts sequences for “CH1” that find use in embodiments of TSLPR x CD3 bsAbs.

FIG. 21 depicts sequences for “hinge” that find use in embodiments of TSLPR x CD3 bsAbs.

FIG. 22 depicts the constant domain of the cognate light chains that find use in the subject TSLPR x CD3 bsAbs that utilize a Fab binding domain.

FIG. 23 depicts a couple of formats of the present invention. FIG. 23A depicts the “1+1 Fab-scFv-Fc” format, with a first Fab arm binding TSLPR and a second scFv arm binding CD3. FIG. 23B depicts the “2+1 Fab2-scFv-Fc” format, with a first Fab arm binding TSLPR and a second Fab-scFv arm, wherein the Fab binds TSLPR and the scFv binds CD3.

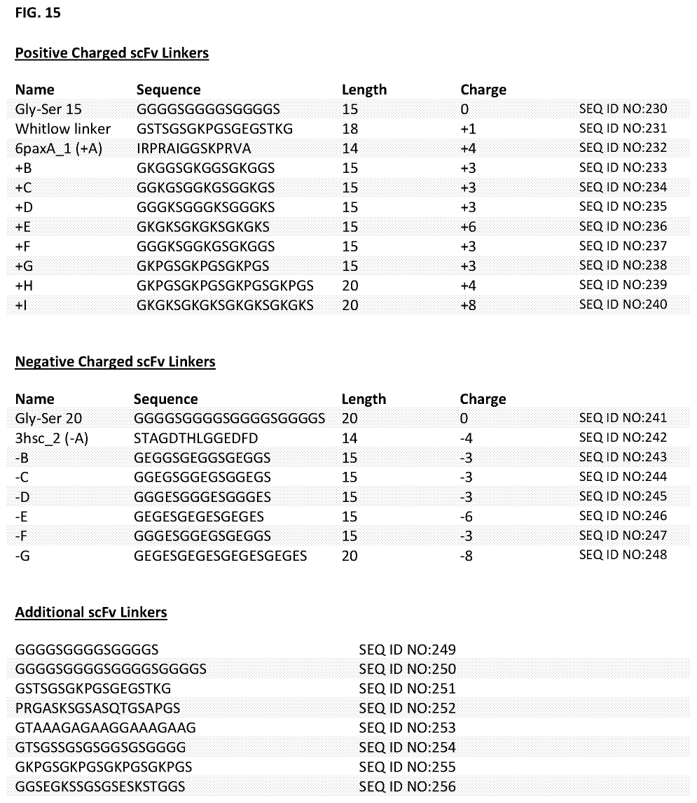

FIGS. 24A-24E depict sequences for exemplary anti-CD3 scFvs suitable for use in the bispecific antibodies of the invention. The CDRs are underlined, the scFv linker is double underlined (in the sequences, the scFv linker is a positively charged scFv (GKPGS)₄ linker (SEQ ID NO: XXX), although as will be appreciated by those in the art, this linker can be replaced by other linkers, including uncharged or negatively charged linkers, some of which are depicted in FIG. 15), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 25 depicts sequences for illustrative bivalent TSLPR mAb using the 1B7 binding domain.

FIGS. 26A-26G depict sequences for illustrative TSLPR x CD3 bsAb in the 1+1 Fab-scFv-Fc and 2+1 Fab2-scFv-Fc formats and the 1B7 TSLPR binding domain. Slashes indicate the border(s) between the variable regions (e.g. VH and VL) and other domains (e.g. linkers and constant domains). It should be noted that the TSLPR x CD3 bsAbs can utilize variable regions, Fc region, and constant domain sequences that are 90, 95, 98 and 99% identical (as defined herein), and/or contain from 1, 2, 3, 4, 5, 6, 7, 8, 9 or 10 amino acid substitutions.

FIG. 27 depicts binding of illustrative TSLPR x CD3 bispecific antibodies XENP39597 (1+1; CD3-High [VHVL]) and XENP39599 (2+1; CD3-Hight-Int #1[VL VH]) and bivalent TSLPR mAb to U266B1 and SNU423 cells.

FIGS. 28A and 28B depict induction of RTCC on A) U266B1 and B) SNU423 cells by illustrative TSLPR x CD3 bispecific antibodies XENP39597 (1+1; CD3-High [VHVL]), XENP39598 (1+1; CD3-Hight-Int #1[VHVL], and XENP39599 (2+1; CD3-Hight-Int #1[VLVH]). 20K U266B1 or SNU423 cells were incubated with 200K T cells (isolated from PBMC) with various concentrations of TSLPR x CD3 bispecific antibodies at 37° C. for 48 hours.

FIGS. 29A-29C depict induction of RTCC on A) U266B1, B) MHH-CALL-4, and C) MUTZ5 cells by illustrative TSLPR x CD3 bispecific antibodies XENP39597 (1+1; CD3-High [VHVL]), XENP39598 (1+1; CD3-Hight-Int #1[VHVL], and XENP39599 (2+1; CD3-Hight-Int #1[VLVH]) and negative control RSV x CD3 bispecific antibodies XENP13245 (1+1; CD3-High [VHVL]) and XENP32140 (2+1; CD3-High-Int #1[VLVH]). 20K U266B1, MHH-CALL-4, or MUTZ5 cells were incubated with human PBMCs (1:1 effector:target) with various concentrations of TSLPR x CD3 bispecific antibodies at 37° C. for 24 hours.

FIG. 30 depicts induction of RTCC on A) Reh/TSLPR-Low (55K antigen density), B) Reh/TSLPR-Medium (100K antigen density), C) Reh/TSLPR-High (160K antigen density), and D) Reh/TSLPR-parental (0.4K antigen density) cells by illustrative TSLPR x CD3 bispecific antibodies XENP39597 (1+1; CD3-High [VHVL]), XENP39598 (1+1; CD3-Hight-Int #1[VHVL], and XENP39599 (2+1; CD3-Hight-Int #1[VLVH]) and negative control RSV x CD3 bispecific antibodies XENP13245 (1+1; CD3-High [VHVL]) and XENP32140 (2+1; CD3-High-Int #1[VLVH]). Cells were incubated with T cells isolated from human PBMCs (1:1 effector:target) with various concentrations of TSLPR x CD3 bispecific antibodies at 37° C. for 48 hours.

FIG. 31 depicts induction of RTCC on MUTZ5 cells by illustrative TSLPR x CD3 bispecific antibodies XENP39597 (1+1; CD3-High [VHVL]), XENP39598 (1+1; CD3-Hight-Int #1[VHVL], and XENP40361 (1+1; CD3-Hight-Int #2 [VHVL]). Target cells were incubated with T cells isolated from human PBMCs (1:1 effector:target) with various concentrations of TSLPR x CD3 bispecific antibodies at 37° C. for 48 hours.

FIG. 32 summarizes RTCC EC₅₀ on various cell lines by XENP39597, XENP39598, and XENP39599 as determined by the various experiments.

### DETAILED DESCRIPTION

### I. Overview

The present disclosure provides antibodies and antigen-binding domain compositions capable of binding to thymic stromal lymphopoietin receptor (TSLPR). In one aspect, provided herein are bispecific antibodies that bind thymic stromal lymphopoietin receptor (TSLPR) and CD3 epsilon (CD3e), i.e., anti-TSLPR x anti-CD3e antibodies. In embodiments, these anti-TSLPR x anti-CD3e antibodies are used therapeutic agents for therapies of cancers expressing TSLPR including but not limited to ALL, B-ALL, and, specifically, Ph-like B-ALL. In embodiments, the bispecific antibodies provided herein advantageously have a long in-vivo half-life.

In some aspects, the anti-TSLPR x anti-CD3e antibody binds monovalently to TSLPR on a first cell and binds monovalently to CD3 on a second cell. In some aspects, the bispecific antibody of the invention binds bivalently to TSLPR on a first cell and binds monovalently to CD3 on a second cell.

The anti-TSLPR x anti-CD3e antibodies provided herein are multifunctional and can be bispecific or multispecific. In some embodiments, the antibodies of the invention include two binding moieties and one Fc part derived from normal human IgG. In some aspects, the bispecific antibody simultaneously binds to a cancer cell, for example, a leukemia cell, which expresses or overexpresses TSLPR on the surface and engages a T-cell through binding of CD3e on the T cell surface. In some embodiments, the binding of the anti-TSLPR x anti-CD3e antibody to the cancer cell, for example, the leukemia cell and the T cell leads to T-cell mediated cytotoxicity and results in the killing of the cancer/leukemia cell. The second cell that expresses CD3 on its surface can be a myeloid effector cell, or a T cell. By constructing and using anti-TSLPR x anti-CD3e antibody provided herein, it is possible to recruit myeloid effector cells that express CD3 into close proximity to cancer/leukemia cells that express TSLPR, thereby achieving the purpose of killing the cancer/leukemia cells more effectively.

### II. Nomenclature

The naming nomenclature of particular antigen binding domains (e.g., CD3e binding domains) use a “Hx.xx_Ly.yy” type of format, with the numbers being unique identifiers to particular variable chain sequences. Thus, for example, the CD3e binding domain “[anti-CD3]_H1.30_L1.47” (FIG. 24A) includes a variable heavy domain, H1.30, and a variable light domain L1.47. In the case that these sequences are used as scFvs, the designation “H1.30_L1.47”, indicates that the binding domain includes a variable heavy domain “H1.30” combined with a variable light domain “L1.47,” and is in VH-linker-VL orientation, from N- to C-terminus. This molecule with the identical sequences of the heavy and light variable domains but in the reverse order (VL-linker-VH orientation, from N- to C-terminus) would be designated “L1.47_H1.30”. Similarly, different constructs may “mix and match” the heavy and light chains as will be evident from the sequence listing and the Figures.

### III. Definitions

In order that the application may be more completely understood, several definitions are set forth below. Such definitions are meant to encompass grammatical equivalents.

By “TSLPR,” “TSLP Receptor,” “thymic stromal lymphopoietin receptor,” “CRL2,” and “Cytokine Receptor-Like Factor 2” as used herein is meant the receptor of the cytokine thymic stromal lymphopoietin (SwissProt Q9HC73.1), which receptor is generally expressed on cells of myeloid origin. Unless otherwise stated, the antigen binding domains that bind to human TSLPR binds to the extracellular domain (ECD) of human TSLPR, comprising amino acid residues 25 to 230.

By “CD3” (cluster of differentiation 3) as used herein is meant a protein complex, that is composed of a CD3 gamma (CD3γ) chain (SwissProt P09693), a CD3 delta (CD3δ) chain (SwissProt P04234), CD3 epsilon (CD3ε and CD3e) chains (SwissProt P07766), and a CD3 zeta chain homodimer (SwissProt P20963). These chains associate with the T-cell receptor (TCR) and the (ζ-chain to generate an activation signal in T lymphocytes. The TCR, ζ-chain, and CD3 molecules together comprise the TCR complex. CD3 is expressed on T cells. Where reference is made to CD3 herein, the reference is to human CD3, unless specifically stated otherwise. Unless otherwise stated, the antigen binding domains that bind to human CD3e generally bind to the ECD of humanCD3e, which is at the N-terminus, and in some cases bind to the first 10⁻¹⁵ amino acids of the mature human CD3e protein.

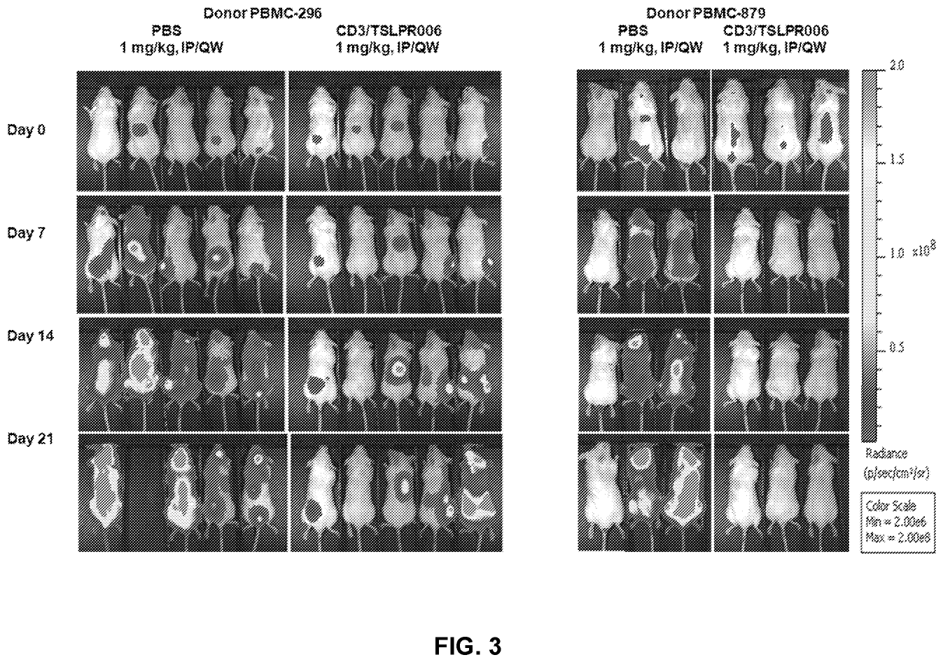

By “ablation” as used herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 3, which generally are added to both monomers.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

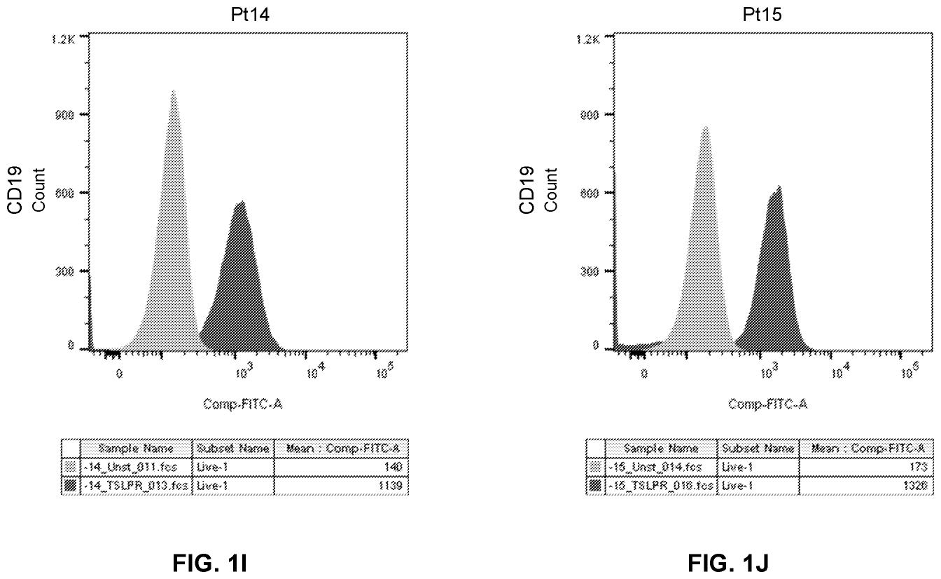

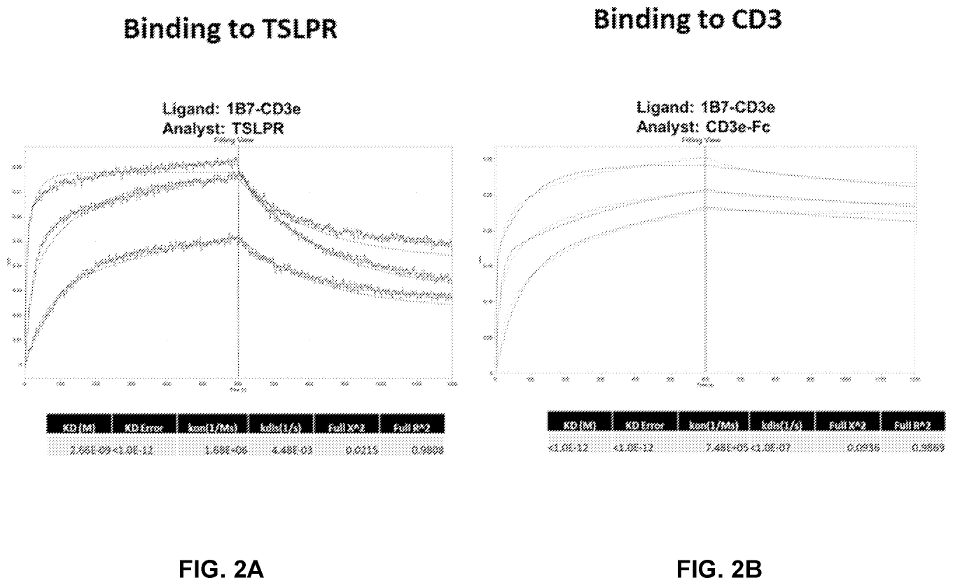

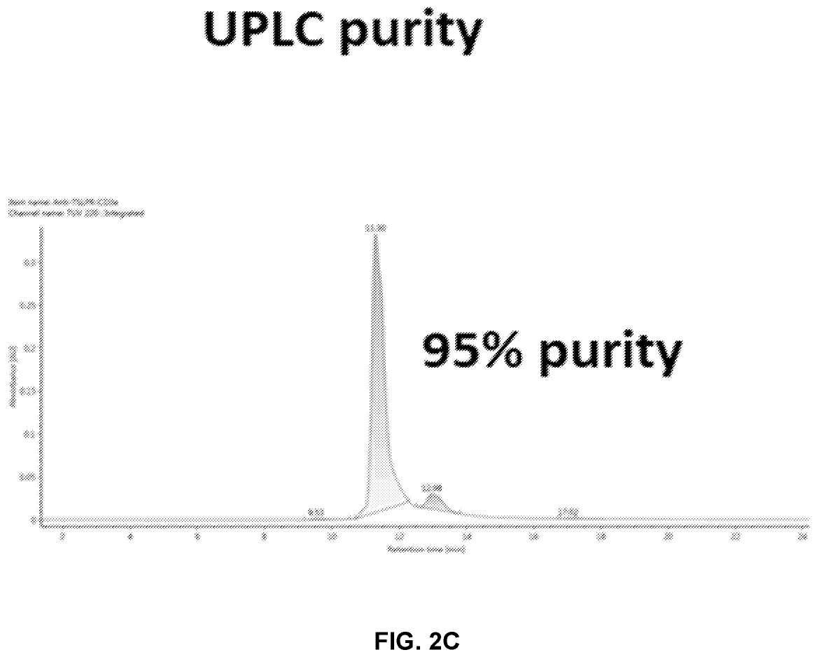

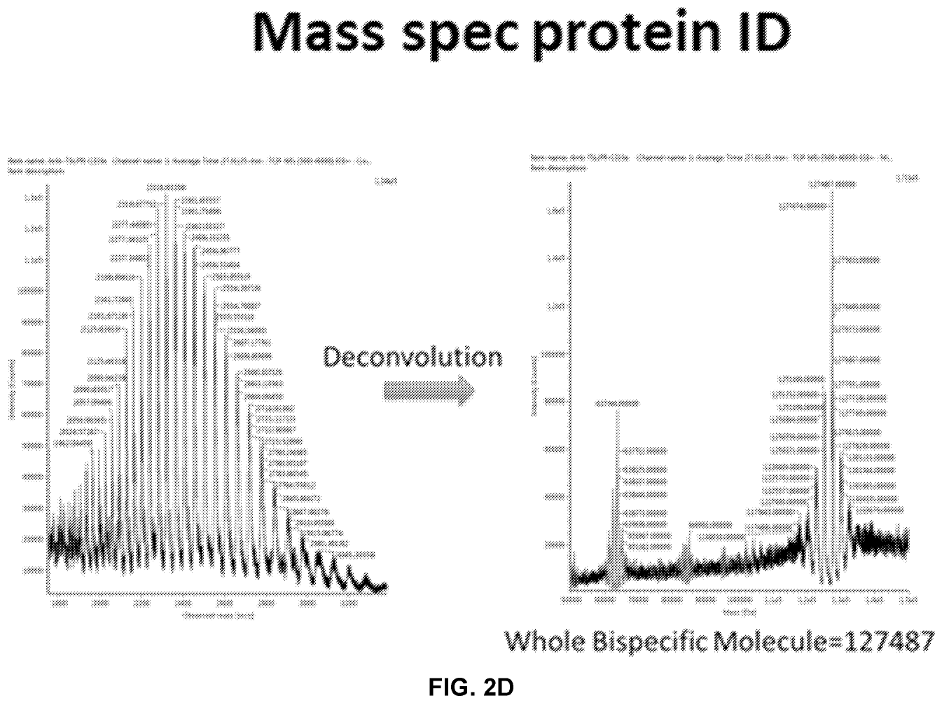

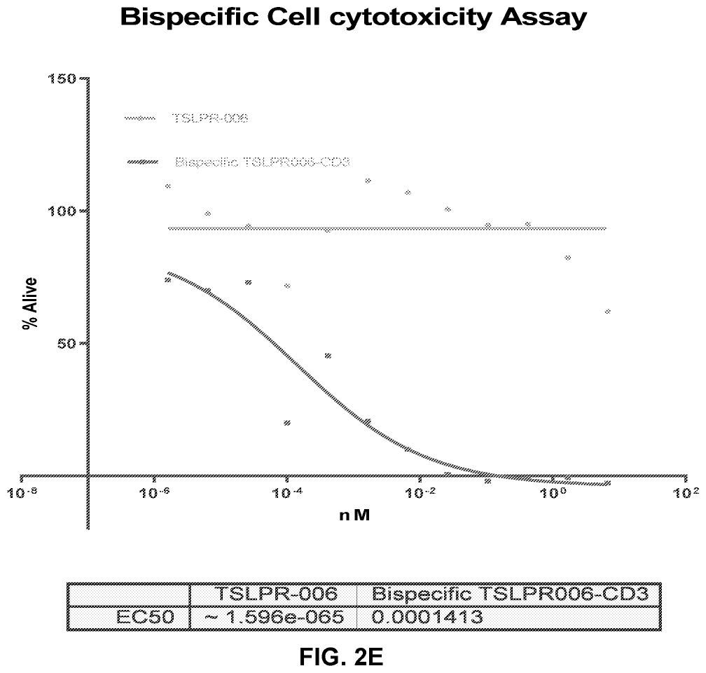

Other useful antibody formats include, but are not limited to, the “1+1 Fab-scFv-Fc,” and “2+1 Fab2-scFv-Fc” formats provided herein (see, e.g., FIG. 23). See also, US20180127501A1, which is incorporated by reference herein, particularly in pertinent part relating to antibody formats (see, e.g., FIG. 2 of US20180127501A1).

Antibody heavy chains typically include a variable heavy domain (VH, also referred to herein as “heavy chain variable region”), which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL, also referred to herein as “light chain variable region”), which includes light chain CDRs vlCDR1-3, and a constant light chain region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., TSLPR or CD3e) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27 (1): 55-77 (2003):

TABLE 2

| | Kabat + Chothia | IMGT | Kabat | AbM | Chothia | Contact | Xencor |
|---|---|---|---|---|---|---|---|
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy domain and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del or E233_ designates a deletion of glutamic acid at position 233. Additionally, EDA233- or EDA233 #designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### IV. Anti-TSLPR Antibodies and Antigen-Binding Fragments Thereof

In some aspects, provided herein are anti-TSLPR antibodies (e.g., anti-TSLPR x anti-CD3e antibodies), and antigen-binding fragments thereof which specifically bind to Thymic Stromal Lymphopoietic (TSLPR) (e.g., human TSLPR). In some aspects, the amino acid sequence for human TSLPR comprises SEQ ID NO: 76 (see UniProtKB/Swiss-Prot Accession No. Q9HC73.1).

In embodiments, the anti-TSLPR antibodies and antigen-binding fragments include a TSLPR binding domain. In some aspects, the TSLPR binding domain comprises six CDRs (e.g., a vhCDR1, a vhCDR2, a vhCDR3, a vlCDR1, a vlCDR2, and a vlCDR3). In some aspects, the CDRs of the antibody or antigen-binding fragment thereof are annotated according to Chothia, Partome, and/or Kabat numbering.

In some aspects, the TSLPR binding domain comprises one variable heavy domain/variable light domain (VH/VL) combination that binds TSLPR. In some aspects, the TSLPR binding domain is a single-chain variant fragment (scFv) that binds TSLPR. In embodiments, the antibodies or antigen-binding fragments thereof provided herein exhibit particularly advantageous properties as compared with other antibodies in the art. Such advantageous properties include, but are not limited to: two high affinity binding sites; a long half-life in vivo due to the Ig-like format; high biological activities with very effective leukemia cell killing (such as B cells) due to effective recruitment of immune effector cells and complement to the Fc regions of the antibodies provided herein; and reduced potential for side effects.

In some aspects, the antibodies have been charge engineered by introducing S293D mutation, 1332E mutation, or both S239D and 1332E mutations in the Fc regions of IgG1 (SEQ ID NO: 226).

### A. TSLPR Binding Domains

Suitable TSLPR binding domains of the anti-TSLPR antibodies or antigen-binding fragments thereof described herein comprise a variable heavy domain (VH) and a light chain variable domain (VL). In embodiments, the VH and VL is assembled in a Fab format of an Ig-like antibody. In embodiments, the VL and VL is assembled in scFv format. In embodiments, the antibodies or antigen-binding fragments thereof of the disclosure comprise two Fab that are linked to Fc regions to form a IgG-like antibody. Other formats of antibodies or antigen-binding fragments thereof described herein include; IgG-like antibodies comprising two scFv arms with binding sites for TSLPR each linked to an Fc portion; IgG-like antibodies with a Fab arm with a binding site for TSLPR and a scFv arm with a binding site for TSLPR each linked to an Fc portion. Further formats include non-IgG-like antibodies or antigen-binding fragments thereof that comprise two Fab arms with binding sites for TSLPR; two scFv arms with binding sites for TSLPR; or one Fab arm with a binding site for TSLPR and one scFv arm with a binding site for TSLPR but no Fc portions. In embodiments, the antibodies or antigen-binding fragments thereof of the disclosure comprise Fc portions.

In embodiments, antibodies or antigen-binding fragments provided herein comprise at least one TSLPR binding domain that binds an extracellular part of TSLPR. In embodiments, antibodies or antigen-binding fragments provided herein comprise two TSLPR binding domain that can bind an extracellular part of TSLPR. In embodiments, the TSLPR binding domains bind TSLPR with a Kd that is between 10⁻⁵ M and 10⁻¹¹ M. In embodiments, the variable domains bind TSLPR with a Kd that is between 106 M and 10⁻¹⁰ M. In embodiments, the Kd is between 10⁻⁷ M and 10⁻⁹ M or between 10⁻⁸ M and 10⁻⁹ M.

In embodiments, the anti-TSLPR antibody or antigen-binding fragment thereof binds to TSLPR with a binding affinity of at least 1×10⁻⁵ M, at least 5×10⁻⁶ M, at least 1×10⁻⁶ M, at least 5×10⁻⁷ M, at least 1×10⁻⁷ M, at least 5×10⁻⁸ M, at least 1×10⁻⁸ M, at least 5×10⁻⁹ M, at least 1×10⁻⁹ M, at least 5×10⁻¹⁰ M, at least 1×10⁻¹⁰ M, at least 5×10⁻¹¹ M, or at least 1×10⁻¹¹ M.

Aspects of the variable heavy domains and variable light domains of the TSLPR binding domain are further discussed in detail below.

### 1. Variable Heavy Domains

In embodiments, the anti-TSLPR antibodies or antigen-binding fragments thereof comprise a human TSLPR binding domain comprising a variable heavy domain, wherein the variable heavy domain comprises:
(a) a vhCDR1 comprising the amino acid sequence SEQ ID NO: 13, a vhCDR2 comprising the amino acid sequence SEQ ID NO: 14, and a vhCDR3 comprising the amino acid sequence SEQ ID NO: 15;
(b) a vhCDR1 comprising the amino acid sequence SEQ ID NO: 16, a vhCDR2 comprising the amino acid sequence SEQ ID NO: 17, and a vhCDR3 comprising the amino acid sequence SEQ ID NO: 18; or
(c) a vhCDR1 comprising the amino acid sequence SEQ ID NO: 19, a vhCDR2 comprising the amino acid sequence SEQ ID NO: 20, and a vhCDR3 comprising the amino acid sequence SEQ ID NO: 21.

In embodiments, the anti-TSLPR antibodies or antigen-binding fragments thereof comprise heavy chain alternate CDRs annotated according to Chothia, Partome, and/or Kabat numbering. In embodiments, the heavy chain CDRs are Custom or Custom2. Exemplary alternate CDRs are provided in Tables 3-5.

TABLE 3
Alternate CDRs for TSLPR-4 Heavy Chain (1A9)
(SEQ ID NO: 9)
TSLPR-4 VH Protein (1A9) SEQ ID NO: 9

| | | | SEQ | | |
|---|---|---|---|---|---|
| | Defi- | Sequence | ID | Resi- | |
| Region | nition | Fragment | NO | dues | Length |
| vhCDR1 | Custom | GYSISSGYYWG | 19 | 26-36 | 11 |
| | Paratome | YSISSGYYWG | 52 | 27-36 | 10 |
| | Chothia | GYSISSGY | 53 | 26-33 | 8 |
| | Custom2 | YSISSGYY | 220 | 27-34 | 8 |
| vhCDR2 | Custom | RIYHSGTTSYNPSLKR | 20 | 51-66 | 16 |
| | Paratome | WIGRIYHSGTTSY | 54 | 48-60 | 13 |
| | Chothia | YHSGT | 55 | 53-57 | 5 |
| vhCDR3 | Custom | NWGGNWFDP | 21 | 99-107 | 9 |
| | Paratome | RNWGGNWFDP | 56 | 98-107 | 10 |
| | Chothia | NWGGNWFDP | 57 | 99-107 | 9 |
| | Custom2 | ARNWGGNWFDP | 221 | 97-107 | 11 |

TABLE 4
Alternate CDRs for TSLPR-6 Heavy Chain (1B7)
(SEQ ID NO: 7)
TSLPR-6 VH Protein (1B7) SEQ ID NO: 7

| | | | SEQ | | |
|---|---|---|---|---|---|
| | Defi- | Sequence | ID | Resi- | |
| Region | nition | Fragment | NO | dues | Length |
| vhCDR1 | Custom | GGSISSSNWWS | 13 | 26-36 | 11 |
| | Paratome | GSISSSNWWS | 40 | 27-36 | 10 |
| | Chothia | GGSISSSN | 41 | 26-33 | 8 |
| vhCDR2 | Custom | EIYHGGTTNNNPSLKS | 14 | 51-66 | 16 |
| | Paratome | WIGEIYHGGTTNN | 42 | 48-60 | 13 |
| | Chothia | YHGGT | 43 | 53-57 | 5 |
| vhCDR3 | Custom | HRYSNGLDY | 15 | 99-107 | 9 |
| | Paratome | RHRYSNGLDY | 44 | 98-107 | 10 |
| | Chothia | HRYSNGLDY | 45 | 99-107 | 9 |
| | Custom2 | ARHRYSNGLDY | 222 | 97-107 | 11 |

TABLE 5
Alternate CDRs for TSLPR-21 Heavy Chain (1C3)
(SEQ ID NO: 8)
TSLPR-21 VH Protein (1C3) SEQ ID NO: 8

| | | | SEQ | | |
|---|---|---|---|---|---|
| | Defi- | Sequence | ID | Resi- | |
| Region | nition | Fragment | NO | dues | Length |
| vhCDR1 | Custom | GYTFSGYYMH | 16 | 26-35 | 10 |
| | Paratome | YTFSGYYMH | 64 | 27-35 | 9 |
| | Chothia | GYTFSGY | 65 | 26-32 | 7 |
| vhCDR2 | Custom | RFNLHGGVADYAQKFQ | 17 | 50-65 | 16 |
| | Paratome | WMGRFNLHGGVADYA | 66 | 47-61 | 15 |
| | Chothia | NLHGGV | 67 | 52-57 | 6 |
| | Custom2 | RFNLHGGVADYAQKFQG | 223 | 50-66 | 17 |
| vhCDR3 | Custom | ERQWLVQDYFDY | 18 | 98-109 | 12 |
| | Paratome | VRERQWLVQDYFDY | 68 | 96-109 | 14 |
| | Chothia | ERQWLVQDYFDY | 69 | 98-109 | 12 |
| | Custom2 | ARERQWLVQDYFDY | 224 | 98-109 | 14 |

In embodiments, the anti-TSLPR antibodies or antigen-binding fragments thereof comprise a variable heavy domain as disclosed in SEQ ID NOs: 76-93.

In embodiments, the anti-TSLPR antibodies or antigen-binding fragments thereof comprise heavy chain CDRs as disclosed in Table 9.

Conservative variations of 1, 2, or 3 amino acid residues from the recited CDR sequences are allowed while retaining the same kind of binding activity (in kind, not necessarily in amount). Hence, the heavy chain CDR 1, 2 and 3 sequences preferably contain sequences that deviate in no more than three, preferably no more than two, preferably no more than one amino acid from the recited CDR sequences. In certain aspects, the heavy chain CDR 1, 2 and 3 sequences are identical to the recited CDR sequences.

In embodiments, the anti-TSLPR antibody or antigen-binding fragment thereof comprises a human TSLPR binding domain comprising an variable heavy domain that comprises SEQ ID NO: 7, 8, 9, or 76-93.

In embodiments, the anti-TSLPR antibody or antigen-binding fragment thereof comprises a human TSLPR binding domain comprising an variable heavy domain that comprises an amino acid sequence set forth in SEQ ID NO: 7, 8, 9, or 76-93.

In embodiments, the anti-TSLPR antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 7.

In embodiments, the anti-TSLPR antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 8.

In embodiments, the anti-TSLPR antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 9.

In embodiments, the anti-TSLPR antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 76-93.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and wherein the variable heavy domain is at least 80% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR2 selected from SEQ ID NO: 14, 17, 20, 42, 43, 54, 55, 66, and 67 and wherein the variable heavy domain is at least 80% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR3 selected from SEQ ID NO: 15, 18, 21, 44, 45, 56, 57, 68, and 69 and wherein the variable heavy domain is at least 80% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and wherein the variable heavy domain is at least 85% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR2 selected from SEQ ID NO: 14, 17, 20, 42, 43, 54, 55, 66, and 67 and wherein the variable heavy domain is at least 85% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR3 selected from SEQ ID NO: 15, 18, 21, 44, 45, 56, 57, 68, and 69 and wherein the variable heavy domain is at least 85% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and wherein the variable heavy domain is at least 90% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR2 selected from SEQ ID NO: 14, 17, 20, 42, 43, 54, 55, 66, and 67 and wherein the variable heavy domain is at least 90% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR3 selected from SEQ ID NO: 15, 18, 21, 44, 45, 56, 57, 68, and 69 and wherein the variable heavy domain is at least 90% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and wherein the variable heavy domain is at least 95% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR2 selected from SEQ ID NO: 14, 17, 20, 42, 43, 54, 55, 66, and 67 and wherein the variable heavy domain is at least 95% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR3 selected from SEQ ID NO: 15, 18, 21, 44, 45, 56, 57, 68, and 69 and wherein the variable heavy domain is at least 95% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and wherein the variable heavy domain is at least 96% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR2 selected from SEQ ID NO: 14, 17, 20, 42, 43, 54, 55, 66, and 67 and wherein the variable heavy domain is at least 96% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR3 selected from SEQ ID NO: 15, 18, 21, 44, 45, 56, 57, 68, and 69 and wherein the variable heavy domain is at least 96% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and wherein the variable heavy domain is at least 97% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR2 selected from SEQ ID NO: 14, 17, 20, 42, 43, 54, 55, 66, and 67 and wherein the variable heavy domain is at least 97% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR3 selected from SEQ ID NO: 15, 18, 21, 44, 45, 56, 57, 68, and 69 and wherein the variable heavy domain is at least 97% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and wherein the variable heavy domain is at least 98% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR2 selected from SEQ ID NO: 14, 17, 20, 42, 43, 54, 55, 66, and 67 and wherein the variable heavy domain is at least 98% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR3 selected from SEQ ID NO: 15, 18, 21, 44, 45, 56, 57, 68, and 69 and wherein the variable heavy domain is at least 98% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 80% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 85% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 90% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 91% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 92% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 93% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 94% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 95% sequence identical to an amino acid sequence selected from SEQ ID NO: 7, 8, and 9.

In embodiments, the TSLPR binding domain is a human TSLPR binding domain that comprises a variable heavy domain, wherein the variable heavy domain comprises an amino acid sequence that comprises a vhCDR1 selected from SEQ ID NO: 13, 16, 19, 40, 41, 52, 53, 64, and 65 and a vhCDR2 selected from SEQ ID NO: 14, 17, 20, 42, 43, 54, 55, 66, and 67 and a vhCDR3 selected from SEQ ID NO: 15, 18, 21, 44, 45, 56, 57, 68, and 69, wherein the variable heavy domain is at least 96% sequence identical to an amino acid sequence selected from SEQ ID NO: 7, 8, and 9.

In embodiments, the antibody or antigen-binding fragment thereof of the disclosure comprises an amino acid sequence set forth in SEQ ID NO: 7, 8, 9, or 76-93.

In embodiments, the antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 7.

In embodiments, the antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 8.

In embodiments, the antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 9.

In embodiments, the antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 76-93.

In embodiments, the antibody or antigen-binding fragment thereof comprises a variable heavy domain that comprises an amino acid sequence that comprises a vhCDR1 selected from SEQ ID NO: 13, 16, 19, 40, 41, 52, 53, 64, and 65 and wherein the variable heavy domain is at least 80% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR2 selected from SEQ ID NO: 14, 17, 20, 42, 43, 54, 55, 66, and 67 and wherein the variable heavy domain is at least 80% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR3 selected from SEQ ID NO: 15, 18, 21, 44, 45, 56, 57, 68, and 69 and wherein the variable heavy domain is at least 80% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR1 selected from SEQ ID NO: 13, 16, 19, 40, 41, 52, 53, 64, and 65 and wherein the variable heavy domain is at least 85% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR2 selected from SEQ ID NO: 14, 17, 20, 42, 43, 54, 55, 66, and 67 and wherein the variable heavy domain is at least 85% sequence identical to an amino acid sequence selected from SEQ ID NO: 7, 8, and 9.

In embodiments, antibody or antigen-binding fragment thereof comprises a variable heavy domain that comprises an amino acid sequence that comprises a vhCDR3 selected from SEQ ID NO: 15, 18, 21, 44, 45, 56, 57, 68, and 69 and wherein the variable heavy domain is at least 85% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR1 selected from SEQ ID NO: 13, 16, 19, 40, 41, 52, 53, 64, and 65 and wherein the variable heavy domain is at least 90% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR2 selected from SEQ ID NO: 14, 17, 20, 42, 43, 54, 55, 66, and 67 and wherein the variable heavy domain is at least 90% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR3 selected from SEQ ID NO: 15, 18, 21, 44, 45, 56, 57, 68, and 69 and wherein the variable heavy domain is at least 90% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR1 selected from SEQ ID NO: 13, 16, 19, 40, 41, 52, 53, 64, and 65 and wherein the variable heavy domain is at least 95% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR2 selected from SEQ ID NO: 14, 17, 20, 42, 43, 54, 55, 66, and 67 and wherein the variable heavy domain is at least 95% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR3 selected from SEQ ID NO: 15, 18, 21, 44, 45, 56, 57, 68, and 69 and wherein the variable heavy domain is at least 95% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR1 selected from SEQ ID NO: 13, 16, 19, 40, 41, 52, 53, 64, and 65 and wherein the variable heavy domain is at least 96% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR2 selected from SEQ ID NO: 14, 17, 20, 42, 43, 54, 55, 66, and 67 and wherein the variable heavy domain is at least 96% sequence identical to an amino acid sequence selected from SEQ ID NO: 7, 8, and 9.

In embodiments, the antibody or antigen-binding fragment thereof comprises a variable heavy domain that comprises an amino acid sequence that comprises a vhCDR3 selected from SEQ ID NO: 15, 18, 21, 44, 45, 56, 57, 68, and 69 and wherein the variable heavy domain is at least 96% sequence identical to an amino acid sequence selected from SEQ ID NO: 7, 8, and 9.

Variations of 1, 2 or 3 amino acid residues from the recited CDR sequences are allowed while retaining the same kind of binding activity (in kind, not necessarily in amount). Hence, the heavy chain CDR 1, 2 and 3 sequences preferably contain sequences that deviate in no more than three, preferably no more than two, more preferably no more than one amino acid from the recited CDR sequences. In certain aspects, the heavy chain CDR 1, 2 and 3 sequences are identical to the recited CDR sequences.

In embodiments, the variable heavy domain of the antibody or antigen-binding fragment thereof that binds human TSLPR can have from 0 to 10, preferably from 0 to 5 amino acid insertions, deletions, substitutions, additions in the sequence of the variable heavy domain outside of the three CDR sequences, or a combination thereof. In embodiments, the variable heavy domain comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In certain aspects, the TSLPR binding domain is a human TSLPR binding domain that comprises a variable heavy domain, wherein the variable heavy domain comprises:
(i) a vhCDR1 comprising the amino acid sequence SEQ ID NO: 13, a vhCDR2 comprising the amino acid sequence SEQ ID NO: 14, and a vhCDR3 comprising the amino acid sequence SEQ ID NO: 15;
(ii) a vhCDR1 comprising the amino acid sequence SEQ ID NO: 16, a vhCDR2 comprising the amino acid sequence SEQ ID NO: 17, and a vhCDR3 comprising the amino acid sequence SEQ ID NO: 18; or
(iii) a vhCDR1 comprising the amino acid sequence SEQ ID NO: 19, a vhCDR2 comprising the amino acid sequence SEQ ID NO: 20, and a vhCDR3 comprising the amino acid sequence SEQ ID NO: 21.

In certain aspects, the TSLPR binding domain is a human TSLPR binding domain that comprises a variable heavy domain, wherein the variable heavy domain comprises:
(i) a vhCDR1 comprising the amino acid sequence SEQ ID NO: 40, a vhCDR2 comprising the amino acid sequence SEQ ID NO: 42, and a vhCDR3 comprising the amino acid sequence SEQ ID NO: 44;
(ii) a vhCDR1 comprising the amino acid sequence SEQ ID NO: 64, a vhCDR2 comprising the amino acid sequence SEQ ID NO: 66, and a vhCDR3 comprising the amino acid sequence SEQ ID NO: 68; or
(iii) a vhCDR1 comprising the amino acid sequence SEQ ID NO: 52, a vhCDR2 comprising the amino acid sequence SEQ ID NO: 54, and a vhCDR3 comprising the amino acid sequence SEQ ID NO: 56.

In certain aspects, the TSLPR binding domain is a human TSLPR binding domain that comprises a variable heavy domain, wherein the variable heavy domain comprises:
(i) a vhCDR1 comprising the amino acid sequence SEQ ID NO: 41, a vhCDR2 comprising the amino acid sequence SEQ ID NO: 43, and a vhCDR3 comprising the amino acid sequence SEQ ID NO: 45;
(ii) a vhCDR1 comprising the amino acid sequence SEQ ID NO: 65, a vhCDR2 comprising the amino acid sequence SEQ ID NO: 67, and a vhCDR3 comprising the amino acid sequence SEQ ID NO: 69; or
(iii) a vhCDR1 comprising the amino acid sequence SEQ ID NO: 53, a vhCDR2 comprising the amino acid sequence SEQ ID NO: 55, and a vhCDR3 comprising the amino acid sequence SEQ ID NO: 57.

In certain embodiments, the TSLPR binding domain is a human TSLPR binding domain that comprises a variable heavy domain, wherein the VH region comprises a vhCDR1 comprising the amino acid sequence SEQ ID NO: 13, a vhCDR2 comprising the amino acid sequence SEQ ID NO: 14, and a vhCDR3 comprising the amino acid sequence SEQ ID NO: 15.

In certain embodiments, the TSLPR binding domain is a human TSLPR binding domain that comprises a variable heavy domain, wherein the amino acid sequence of the VH region is selected from SEQ ID NO: 7, 8, and 9.

In some embodiments, the variable heavy domain is encoded by a polynucleotide comprising SEQ ID NO: 34, 35, or 36.

### 2. Variable Light Domains

In embodiments, the anti-TSLPR antibody or antigen-binding fragment thereof comprises a variable light domain that binds human TSLPR, wherein the variable light domain comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to an amino acid sequence comprising SEQ ID NO: 10.

In embodiments, the TSLPR binding domain is a human TSLPR binding domain that comprises a variable light domain, wherein the variable light domain comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to an amino acid sequence comprising SEQ ID NO: 11.

In embodiments, the TSLPR binding domain is a human TSLPR binding domain that comprises a variable light domain, wherein the variable light domain comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to an amino acid sequence comprising SEQ ID NO: 12.

In embodiments, the TSLPR binding domain is a human TSLPR binding domain that comprises a variable light domain, wherein the variable light domain comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 10.

In embodiments, the TSLPR binding domain is a human TSLPR binding domain that comprises a variable light domain, wherein the variable light domain comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 11.

In embodiments, the TSLPR binding domain is a human TSLPR binding domain that comprises a variable light domain, wherein the variable light domain comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 12.

In embodiments, the anti-TSLPR antibody or antigen-binding fragment thereof comprises light chain alternate CDRs annotated according to Chothia, Partome, and/or Kabat numbering. Exemplary alternate CDRs are provided in Tables 6-8.

TABLE 6
Alternate CDRs for TSLPR-4 Light Chain (1A9)
(SEQ ID NO: 12)
TSLPR-4 VL Protein (1A9) SEQ ID NO: 12

| | | | SEQ | | |
|---|---|---|---|---|---|
| | Defi- | Sequence | ID | Resi- | |
| Region | nition | Fragment | NO | dues | Length |
| VlCDR1 | Custom | RASQSVSSYLA | 28 | 24-34 | 11 |
| | Paratome | QSVSSYLA | 58 | 27-34 | 8 |
| | Chothia | RASQSVSSYLA | 59 | 24-34 | 11 |
| VlCDR2 | Custom | DASNRAT | 29 | 50-56 | 7 |
| | Paratome | LLIYDASNRAT | 60 | 46-56 | 11 |
| | Chothia | DASNRAT | 61 | 50-56 | 7 |
| VlCDR3 | Custom | QQRSTWLT | 30 | 89-96 | 8 |
| | Paratome | QQRSTWL | 62 | 89-95 | 7 |
| | Chothia | QQRSTWLT | 63 | 89-96 | 8 |

TABLE 7
Alternate CDRs for TSLPR-6 Light Chain (1B7)
(SEQ ID NO: 10)
TSLPR-6 VL Protein (1B7) SEQ ID NO: 10

| | | | SEQ | | |
|---|---|---|---|---|---|
| | Defi- | Sequence | ID | Resi- | |
| Region | nition | Fragment | NO | dues | Length |
| VlCDR1 | Custom | RASQSISNWLA | 22 | 24-34 | 11 |
| | Paratome | QSISNWLA | 46 | 27-34 | 8 |
| | Chothia | RASQSISNWLA | 47 | 24-34 | 11 |
| VlCDR2 | Custom | KASSLES | 23 | 50-56 | 7 |
| | Paratome | LLIYKASSLES | 48 | 46-56 | 11 |
| | Chothia | KASSLES | 49 | 50-56 | 7 |
| VlCDR3 | Custom | QQYNHYST | 24 | 89-96 | 8 |
| | Paratome | QQYNHYS | 50 | 89-95 | 7 |
| | Chothia | QQYNHYST | 51 | 89-96 | 8 |

TABLE 8
Alternate CDRs for TSLPR-21 Light Chain (1C3)
(SEQ ID NO: 11)
TSLPR-21 VL Protein (1C3) SEQ ID NO: 11

| | | | SEQ | | |
|---|---|---|---|---|---|
| | Defi- | Sequence | ID | Resi- | |
| Region | nition | Fragment | NO | dues | Length |
| VlCDR1 | Custom | RASQSISRSLN | 25 | 24-34 | 11 |
| | Paratome | QSISRSLN | 70 | 27-34 | 8 |
| | Chothia | RASQSISRSLN | 71 | 24-34 | 11 |
| VlCDR2 | Custom | AASSLQS | 26 | 50-56 | 7 |
| | Paratome | LLIYAASSLQS | 72 | 46-56 | 11 |
| | Chothia | AASSLQS | 73 | 50-56 | 7 |
| VlCDR3 | Custom | QQSYSISVT | 27 | 89-97 | 9 |
| | Paratome | QQSYSISV | 74 | 89-96 | 8 |
| | Chothia | QQSYSISVT | 75 | 89-97 | 9 |

In embodiments, the anti-TSLPR antibodies or antigen-binding fragments thereof comprise a variable light domain as disclosed in SEQ ID NO: 148-165.

As will be appreciated in the art, the junction between the VL and CL has been defined differently, including starting at position 110 (EU numbering) or position 108 (EU numbering), and the variable light chain sequences herein can include V, VA, VAA or VAAP. SEQ ID NO: 12 includes amino acids VAAP from the light chain constant domain. The variable light chain of TSLPR antibody 1A9 does not include amino acids VAAP from the light chain constant domain CLI region and is disclosed in SEQ ID NO: 227.

SEQ ID NO: 10 includes amino acids VAAP from the light chain constant domain. The variable light chain of TSLPR antibody 1B7 does not include amino acids VAAP from the light chain constant domain CLI region and is disclosed in SEQ ID NO: 228.

SEQ ID NO: 11 includes amino acids VAAP from the light chain constant domain. The variable light chain of TSLPR antibody 1C3 does not include amino acids VAAP from the light chain constant domain CLI region and is disclosed in SEQ ID NO: 229.

In embodiments, the anti-TSLPR antibodies or antigen-binding fragments thereof comprise light chain CDRs as disclosed in Table 10.

Conservative variations of 1, 2, or 3 amino acid residues from the recited CDR sequences are allowed while retaining the same kind of binding activity (in kind, not necessarily in amount). Hence, the light chain CDR 1, 2, and 3 sequences preferably contain sequences that deviate in no more than three, preferably no more than two, preferably no more than one amino acid from the recited CDR sequences. In certain aspects, the light chain CDR 1, 2 and 3 sequences are identical to the recited CDR sequences.

In embodiments, the TSLPR binding domain is a human TSLPR binding domain that comprises a variable light domain and comprises an amino acid sequence comprising SEQ ID NO: 10, 11, 12, 148-165, or 227-229.

In embodiments, the TSLPR binding domain is a human TSLPR binding domain that comprises a variable light domain, wherein the variable light domain comprises an amino acid sequence set forth in SEQ ID NO: 10, 11, 12, 148-165, or 227-229.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 80% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 80% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 80% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 85% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 85% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 85% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 90% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 90% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 90% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 95% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 95% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 95% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 96% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 96% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 96% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 97% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 97% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 97% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 98% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 98% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 98% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 80% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 85% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 90% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 91% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 92% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 93% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 94% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 95% sequence identical to an amino acid sequence selected from SEQ ID NO: 10, 11 and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and a vlCDR2 selected from SEQ ID NO: 23, 26, 29, 48, 49, 60, 61, 72, and 73 and a vlCDR3 selected from SEQ ID NO: 24, 27, 30, 50, 51, 62, 63, 74, and 75, wherein the variable light domain is at least 96% sequence identical to an amino acid sequence selected from SEQ ID NO: 10, 11 and 12.

In embodiments, the antibody or antigen-binding fragment thereof of the disclosure comprises an amino acid sequence set forth in SEQ ID NO: 10, 11, 12, 148-165, or 227-229.

In embodiments, the antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 10.

In embodiments, the antibody or antigen-binding fragment thereof comprises an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 11.

In embodiments, the antibody or antigen-binding fragment thereof an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 12.

In embodiments, the antibody or antigen-binding fragment thereof an amino acid sequence that is at least 85%, 86%, 87%, 88%, 89%, 90%, 91%, 92%, 93%, 94%, 95%, 95.5%, 96%, 96.5%, 97%, 97.5%, 98%, 98.5%, 99%, or 99.5% identical to the amino acid sequence set forth in SEQ ID NO: 148-165 or 227-229.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 80% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 80% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 80% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 85% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 85% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 85% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 90% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 90% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 90% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 95% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 95% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 95% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 96% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 96% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 96% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 97% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 97% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 97% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR1 selected from SEQ ID NO: 22, 25, 28, 46, 47, 58, 59, 70, and 71 and wherein the variable light domain is at least 98% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR2 selected from SEQ ID NO: 23, 26, 29, 48, 49, 60, 61, 72, and 73 and wherein the variable light domain is at least 98% sequence identical to an amino acid sequence selected from SEQ ID NO: 10, 11, and 12.

In embodiments, the TSLPR binding domain is a human TSLPR binding domain that comprises a variable light domain, wherein the variable light domain comprises an amino acid sequence that comprises a vlCDR3 selected from SEQ ID NO: 24, 27, 30, 50, 51, 62, 63, 74, and 75 and wherein the variable light domain is at least 98% sequence identical to an amino acid sequence selected from SEQ ID NO: 10, 11, and 12.

Variations of 1, 2 or 3 amino acid residues from the recited CDR sequences are allowed while retaining the same kind of binding activity (in kind, not necessarily in amount). Hence, the light chain CDR 1, 2 and 3 sequences preferably contain sequences that deviate in no more than three, preferably no more than two, more preferably no more than one amino acid from the recited CDR sequences. In certain aspects, the light chain CDR 1, 2 and 3 sequences are identical to the recited CDR sequences.

In embodiments, the variable light domain of the antibody or antigen-binding fragment thereof that binds human TSLPR can have from 0 to 10, preferably from 0 to 5 amino acid insertions, deletions, substitutions, additions in the sequence of the variable light domain outside of the three CDR sequences, or a combination thereof. In embodiments, the variable light domain comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In certain aspects, the TSLPR binding domain is a human TSLPR binding domain that comprises a variable light domain, wherein the VL region comprises an amino acid sequence that is at least 90%, 95%, 95.5%, 96%, 96.5%, 97%, 97.5%, 98%, 98.5%, 99%, 99.5% or 100% to the amino acid sequence of the VL region set forth in SEQ ID NO: 10, 11, and 12.

In certain aspects, the TSLPR binding domain is a human TSLPR binding domain that comprises a variable light domain, wherein the variable light domain comprises:
(i) a vlCDR1 comprising the amino acid sequence SEQ ID NO: 22, a vlCDR2 comprising the amino acid sequence SEQ ID NO: 23, and a vlCDR3 comprising the amino acid sequence SEQ ID NO: 24;
(ii) a vlCDR1 comprising the amino acid sequence SEQ ID NO: 25, a vlCDR2 comprising the amino acid sequence SEQ ID NO: 26, and a vlCDR3 comprising the amino acid sequence SEQ ID NO: 27; or
(iii) a vlCDR1 comprises the amino acid sequence SEQ ID NO: 28, a vlCDR2 comprising the amino acid sequence SEQ ID NO: 29, and a vlCDR3 comprising the amino acid sequence SEQ ID NO: 30.

In certain aspects, the TSLPR binding domain is a human TSLPR binding domain that comprises a variable light domain, wherein the variable light domain comprises:
(i) a vlCDR1 comprising the amino acid sequence SEQ ID NO: 46, a vlCDR2 comprising the amino acid sequence SEQ ID NO: 48, and a vlCDR3 comprising the amino acid sequence SEQ ID NO: 50;
(ii) a vlCDR1 comprising the amino acid sequence SEQ ID NO: 70, a vlCDR2 comprising the amino acid sequence SEQ ID NO: 72, and a vlCDR3 comprising the amino acid sequence SEQ ID NO: 74; or
(iii) a vlCDR1 comprises the amino acid sequence SEQ ID NO: 58, a vlCDR2 comprising the amino acid sequence SEQ ID NO: 60, and a vlCDR3 comprising the amino acid sequence SEQ ID NO: 62.

In certain aspects, the TSLPR binding domain is a human TSLPR binding domain that comprises a variable light domain, wherein the variable light domain comprises:
(i) a vlCDR1 comprising the amino acid sequence SEQ ID NO: 47, a vlCDR2 comprising the amino acid sequence SEQ ID NO: 49, and a vlCDR3 comprising the amino acid sequence SEQ ID NO: 51;
(ii) a vlCDR1 comprising the amino acid sequence SEQ ID NO: 71, a vlCDR2 comprising the amino acid sequence SEQ ID NO: 73, and a vlCDR3 comprising the amino acid sequence SEQ ID NO: 75; or
(iii) a vlCDR1 comprises the amino acid sequence SEQ ID NO: 59, a vlCDR2 comprising the amino acid sequence SEQ ID NO: 61, and a vlCDR3 comprising the amino acid sequence SEQ ID NO: 63.

In embodiments, the variable light chain of one or both VL binding regions of the anti-TSLPR antibody or antigen-binding fragment thereof can have from 0 to 10, preferably from 0 to 5 amino acid insertions, deletions, substitutions, additions or a combination thereof with respect to SEQ ID NO: 10, 11, 12. In embodiments, the variable light domain of one or both VH/VL binding regions of the anti-TSLPR antibody or antigen-binding fragment thereof comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In some embodiments, the variable light domain is encoded by a polynucleotide comprising SEQ ID NO: 37, 38, or 39.

### 3. Variable Heavy Domain/Variable Light Domain Combinations

### B. Constant Regions

As will be appreciated by those in the art, the antigen binding domains of the invention that bind to human TSLPR and human CD3e are fused to antibody constant regions in a variety of ways, depending on the format used. That is, in some cases, traditional bivalent, monospecific antibodies can be made against TSLPR. In other formats, as described below, the ABDs are linked with heterodimerization Fc domains, that can include a number of additional variants (as compared to human IgG1, IgG2 or IgG4), depending on use and format.

In embodiments, the antibodies disclosed herein comprise an IgG1 constant region comprising CH1, hinger, CH2, and CH3. In embodiments, the IgG1 constant region comprises the amino acid sequence of SEQ ID NO: 225.

(SEQ ID NO: 225)
IgG1 (bold letter indicates S239 and I332):
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK.

In embodiments, the antibodies disclosed herein comprise the IgG1 constant region of SEQ ID NO: 225 and comprise the VH and VL of SEQ ID NO: 9 and 12 [Clone 1A9], the VH and VL of SEQ ID NO: 7 and 10 [Clone 1B7], the VH and VL of SEQ ID NO: 8 and 11 [Clone 1C3], the VH and VL of SEQ ID NO: 76 and 148 [Clone 2E4], the VH and VL of SEQ ID NO: 77 and 149 [Clone 2H8], the VH and VL of SEQ ID NO: 78 and 150 [Clone 3E5], the VH and VL of SEQ ID NO: 79 and 151 [Clone 1A10], the VH and VL of SEQ ID NO: 80 and 152 [Clone 1B9], the VH and VL of SEQ ID NO: 81 and 153 [Clone 1D10], the VH and VL of SEQ ID NO: 82 and 154 [Clone 1F6], the VH and VL of SEQ ID NO: 83 and 155 [Clone 1G3], the VH and VL of SEQ ID NO: 84 and 156 [Clone 3D8], the VH and VL of SEQ ID NO: 85 and 157 [Clone 10C4], the VH and VL of SEQ ID NO: 86 and 158 [Clone 2A4], the VH and VL of SEQ ID NO: 87 and 159 [Clone 2B2], the VH and VL of SEQ ID NO: 88 and 160 [Clone 2D11], the VH and VL of SEQ ID NO: 89 and 161 [Clone 2E6], the VH and VL of SEQ ID NO: 90 and 162 [Clone 2F1], the VH and VL of SEQ ID NO: 91 and 163 [Clone 2F5], the VH and VL of SEQ ID NO: 92 and 164 [Clone 2H1], or the VH and VL of SEQ ID NO: 93 and 165 [Clone 2H4].

In embodiments, the antibodies disclosed herein comprise the IgG1 constant region of SEQ ID NO: 225 and comprise the VH and VL of SEQ ID NO: 9 and 12 [Clone 1A9], the VH and VL of SEQ ID NO: 7 and 10 [Clone 1B7], the VH and VL of SEQ ID NO: 8 and 11 [Clone 1C3], the VH and VL of SEQ ID NO: 76 and 148 [Clone 2E4], the VH and VL of SEQ ID NO: 77 and 149 [Clone 2H8], the VH and VL of SEQ ID NO: 78 and 150 [Clone 3E5], the VH and VL of SEQ ID NO: 79 and 151 [Clone 1A10], the VH and VL of SEQ ID NO: 80 and 152 [Clone 1B9], the VH and VL of SEQ ID NO: 81 and 153 [Clone 1D10], the VH and VL of SEQ ID NO: 82 and 154 [Clone 1F6], the VH and VL of SEQ ID NO: 83 and 155 [Clone 1G3], the VH and VL of SEQ ID NO: 84 and 156 [Clone 3D8], the VH and VL of SEQ ID NO: 85 and 157 [Clone 10C4], the VH and VL of SEQ ID NO: 86 and 158 [Clone 2A4], the VH and VL of SEQ ID NO: 87 and 159 [Clone 2B2], the VH and VL of SEQ ID NO: 88 and 160 [Clone 2D11], the VH and VL of SEQ ID NO: 89 and 161 [Clone 2E6], the VH and VL of SEQ ID NO: 90 and 162 [Clone 2F1], the VH and VL of SEQ ID NO: 91 and 163 [Clone 2F5], the VH and VL of SEQ ID NO: 92 and 164 [Clone 2H1], or the VH and VL of SEQ ID NO: 93 and 165 [Clone 2H4].

In embodiments, the antibodies disclosed herein comprise an Fc-modified IgG1 constant region comprising CH1, CH2, and CH3 with a S239D/1332E mutation. In embodiments, the IgG1 constant region comprises the amino acid sequence of SEQ ID NO: 226.

Fc-modified IgG1 (bold letters indicate
S239D/I332E):
(SEQ ID NO: 226)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKV
EPKSCDKTHTCPPCPAPELLGGPDVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPEEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK.

In embodiments the antibodies disclosed herein comprise the IgG1 constant region of SEQ ID NO: 226 and comprise the VH and VL of SEQ ID NO: 9 and 12 [Clone 1A9], the VH and VL of SEQ ID NO: 7 and 10 [Clone 1B7], the VH and VL of SEQ ID NO: 8 and 11 [Clone 1C3], the VH and VL of SEQ ID NO: 76 and 148 [Clone 2E4], the VH and VL of SEQ ID NO: 77 and 149 [Clone 2H8], the VH and VL of SEQ ID NO: 78 and 150 [Clone 3E5], the VH and VL of SEQ ID NO: 79 and 151 [Clone 1A10], the VH and VL of SEQ ID NO: 80 and 152 [Clone 1B9], the VH and VL of SEQ ID NO: 81 and 153 [Clone 1D10], the VH and VL of SEQ ID NO: 82 and 154 [Clone 1F6], the VH and VL of SEQ ID NO: 83 and 155 [Clone 1G3], the VH and VL of SEQ ID NO: 84 and 156 [Clone 3D8], the VH and VL of SEQ ID NO: 85 and 157 [Clone 10C4], the VH and VL of SEQ ID NO: 86 and 158 [Clone 2A4], the VH and VL of SEQ ID NO: 87 and 159 [Clone 2B2], the VH and VL of SEQ ID NO: 88 and 160 [Clone 2D11], the VH and VL of SEQ ID NO: 89 and 161 [Clone 2E6], the VH and VL of SEQ ID NO: 90 and 162 [Clone 2F1], the VH and VL of SEQ ID NO: 91 and 163 [Clone 2F5], the VH and VL of SEQ ID NO: 92 and 164 [Clone 2H1], or the VH and VL of SEQ ID NO: 93 and 165 [Clone 2H4].

### C. Further Structural Aspects of the Antibodies or Antigen-Binding Fragments Thereof

In further aspects, the anti-TSLPR antibody or antigen-binding fragment thereof comprises the heavy chain variable regions and light chain variable regions described above that bind TSLPR. Additional aspects of the constant regions of such binding proteins or antibodies, and methods of making them, are described below.

In one aspect, an antibody or antigen-binding fragment thereof of the disclosure exhibits ADCC activity. In such aspect, the antibody or antigen-binding fragment thereof can have improved ADCC activity. One technique for enhancing ADCC of an antibody is afucosylation. (See for instance Junttila, T. T., K. Parsons, et al. (2010). “Superior In vivo Efficacy of Afucosylated Trastuzumab in the Treatment of HER2-Amplified Breast Cancer” Cancer Research 70 (11): 4481-4489). In some aspects, the antibody or antigen-binding fragment thereof according to the disclosure is afucosylated. Alternatively, or additionally, the antibody or antigen-binding fragment thereof according to the disclosure is glycoengineered and/or mutated to improve Fc binding to low affinity activating FcγRIIIa, and/or to reduce binding to the low affinity inhibitory FcγRIIb. In some aspects, the antibody or antigen-binding fragment thereof of the disclosure is afucosylated in order to enhance ADCC activity. In some aspects, the antibody or antigen-binding fragment thereof of the disclosure comprises a reduced amount of fucosylation of the N-linked carbohydrate structure in the Fc region, when compared to the same antibody produced in a mammalian cell.

In some embodiments, the anti-TSLPR antibodies or antigen binding fragments thereof comprises at least one TSLPR binding domain. In some aspects, the antibodies or antigen-binding fragments thereof comprise two TSLPR binding domains that comprise the same amino acid sequence. In some aspects, the antibodies or antigen-binding fragments thereof comprise two TSLPR binding domain that comprise different amino acid sequences.

In some aspects, the two VH/VL domains of the anti-TSLPR antibodies or antigen-binding fragments thereof comprise two heavy chain variable regions that bind human TSLPR, wherein the two VH regions comprise amino acid sequences that are at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7, 8, 9, or 76-93 and two light chain variable regions that bind human TSLPR, wherein the two VL regions comprise amino acid sequences that are at least 90%, 95%, 95.5%, 96%, 96.5%, 97%, 97.5%, 98%, 98.5%, 99%, 99.5% or 100% to the amino acid sequence of the VL region set forth in SEQ ID NO: 10, 11, 12, 148-165, or 227-229.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7 and one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7 and one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8 and one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9.

In some aspects, the two VH/VL domains of the anti-TSLPR antibodies or antigen-binding fragments thereof comprise two light chain variable regions that bind human TSLPR, wherein one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two light chain variable regions that bind human TSLPR, wherein one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two light chain variable regions that bind human TSLPR, wherein one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11 and one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7 and one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8 or 9; and two light chain variable regions that bind human TSLPR, wherein one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11 or 12.

In some aspects, the two VH/VL domains of the anti-TSLPR antibodies or antigen-binding fragments thereof comprise two heavy chain variable regions that bind human TSLPR, wherein both VH regions comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein both VH regions comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10.

In some aspects, the two VH/VL domains of the anti-TSLPR antibodies or antigen-binding fragments thereof comprise two heavy chain variable regions that bind human TSLPR, wherein both VH regions comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10.

In some aspects, the two VH/VL domains of the anti-TSLPR antibodies or antigen-binding fragments thereof comprise two heavy chain variable regions that bind human TSLPR, wherein both VH regions comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and one heavy chain variable region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein both VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the two VH/VL domains of the anti-TSLPR antibodies or antigen-binding fragments thereof comprise two heavy chain variable regions that bind human TSLPR, wherein both VH regions comprise an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9; and two light chain variable regions that bind human TSLPR, wherein one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11 and one VL regions comprise an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 12.

In some aspects, the anti-TSLPR antibodies or antigen-binding fragments thereof comprising two different heavy chain variable regions that bind TSLPR and/or two different light chain variable regions that bind human TSLPR, wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 7 and one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 11 or 12; or wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 8 and one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 or 12; or wherein one VH region comprises an amino acid sequence that is at least 90%, 95%, 95.5%, 96%, 96.5%, 97%, 97.5%, 98%, 98.5%, 99%, 99.5% or 100% identical to the amino acid sequence of the VH region set forth in SEQ ID NO: 9 and one VL region comprises an amino acid sequence that is at least 90%, 95%, 95.5%, 96%, 96.5%, 97%, 97.5%, 98%, 98.5%, 99%, 99.5% or 100% identical to the amino acid sequence of the VL region set forth in SEQ ID NO: 10 or 11; the anti-TSLPR antibodies or antigen-binding fragments thereof further comprise a heavy chain constant domain (CH1) and a light chain constant domain (LC) that, when expressed in a cell favor assembly of SEQ ID NO: 7 VH with SEQ ID NO: 11 or 12 VL; SEQ ID NO: 8 VH with SEQ ID NO: 10 or 12 VL; and/or SEQ ID NO: 9 VH with SEQ ID NO: 10 or 11 VL over assembly of SEQ ID NO: 7 VH with SEQ ID NO: 10 VL; SEQ ID NO: 8 VH with SEQ ID NO: 11 VL; and/or SEQ ID NO: 9 VH with SEQ ID NO: 12 VL.

In some aspects, the VH/VL chains are assembled in a cell using “knob into hole” CH1 and LC domains. In some aspects, the VH/VL chains are assembled in a cell using charge engineered CH1 and LC domains as disclosed herein.

In some aspects, antibodies or antigen-binding fragments thereof comprise a functional part, derivative and/or analogue of the antibodies or antigen-binding fragments thereof described herein. The variant maintains the binding specificity of the antibody or antigen-binding fragment thereof. The functional part, derivative and/or analogue maintains the binding specificity of the antibody or antigen-binding fragment thereof.

In some aspects, the antibody or antigen-binding fragment thereof of the disclosure is a human or humanized antibody or antigen-binding fragment thereof comprising an antigen-binding site that binds TSLPR, wherein the variable domain comprising the TSLPR binding site comprises a VH CDR3 sequence selected from SEQ ID NO: 15, 18, 21, 44, 45, 56, 57, 68, and 69.

In some aspects, the VH variable region comprising the TSLPR binding site preferably comprises the sequence of the CDR1 region, CDR2 region and the CDR3 region of a VH chain comprising an amino acid of SEQ ID NO: 13-21, 40-45, 52-57, or 64-69.

In some aspects, the VL variable region comprising the TSLPR binding site preferably comprises the sequence of the CDR1 region, CDR2 region and the CDR3 region of a VL chain comprising an amino acid of SEQ ID NO: 22-30, 46-51, 58-63, or 70-75.

In some aspects, the CDRs of a VH chain comprising SEQ ID NO: 13-21, 40-45, 52-57, or 64-69 are grafted on framework regions 1, 2, 3, and/or 4 (FR1, FR2, FR3, and FR4) of a human heavy and light chain immunoglobulin or of an immunoglobulin of another mammal.

In some aspects, provided is a human or humanized antibody or antigen-binding fragment thereof comprising an antigen-binding site that binds TSLPR, wherein the variable domain comprising the TSLPR binding site comprises a VH CDR sequence comprising an amino acid sequence comprising SEQ ID NO: 13-21, 40-45, 52-57, or 64-69 and a VL CDR sequence comprising an amino acid sequence comprising SEQ ID NO: 22-30, 46-51, 58-63, or 70-75.

### V. Anti-TSLPR x Anti-CD3e Antibodies

In one aspect, provided herein are novel anti-TSLPR x anti-CD3e antibodies. In embodiments, the anti-TSLPR x anti-CD3e antibodies are useful for the treatment of a TSLPR related condition (e.g., a B-cell Acute Lymphoblastic Leukemia).

The anti-TSLPR x anti-CD3e antibodies are multivalent and include at least two antigen binding domains (ABDs), wherein at least one antigen binding domain is a TSLPR binding domain and at least one antigen binding domain is a CD3 epsilon (CD3e) binding domain. Any suitable TSLPR binding domain and CD3e binding domain can be included in the subject anti-TSLPR x anti-CD3e antibodies, including, for example, the TSLPR binding domains and CD3e binding domains provided herein.

The antigen binding domains provided herein generally include a variable heavy domain (VH) having a VH-CDR1, VH-CDR-2, and VH-CDR-3; and a variable light domain (VL), and a variable light domain (VL) having a VL-CDR1, VL-CDR-2, and VL-CDR-3.

In addition, as discussed above, the numbering used in the sequence listing and figures for the identification of the CDRs is Kabat, however, different numbering can be used, which will change the amino acid sequences of the CDRs as shown in Table 2.

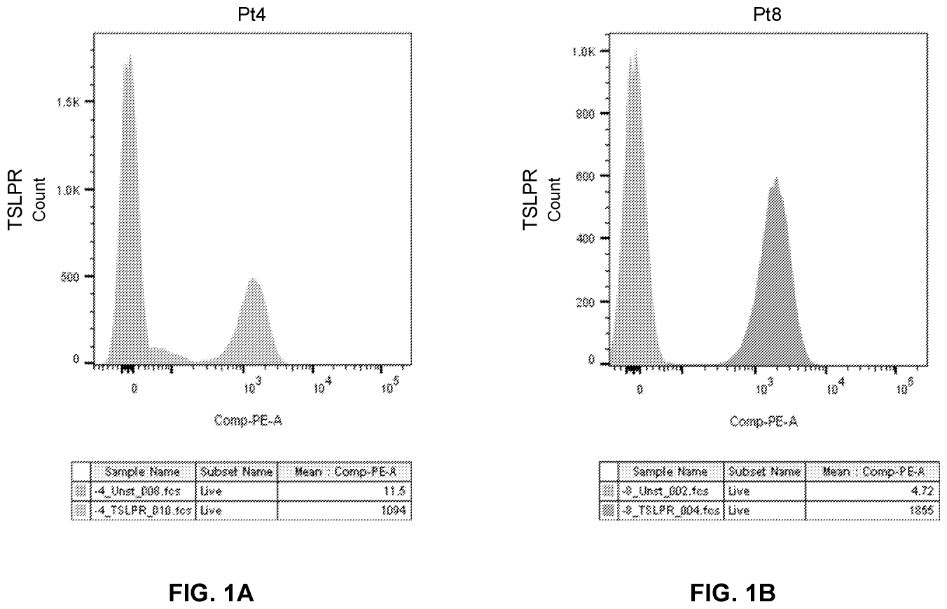

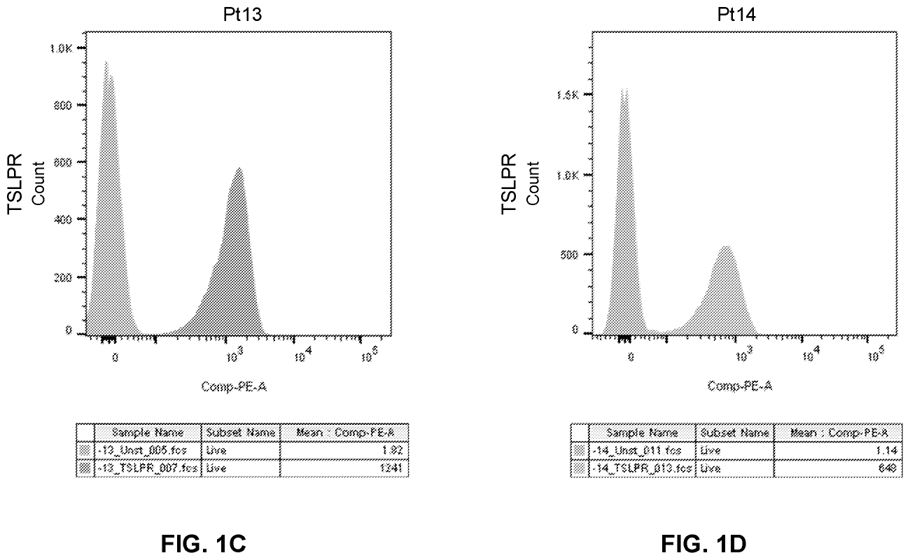

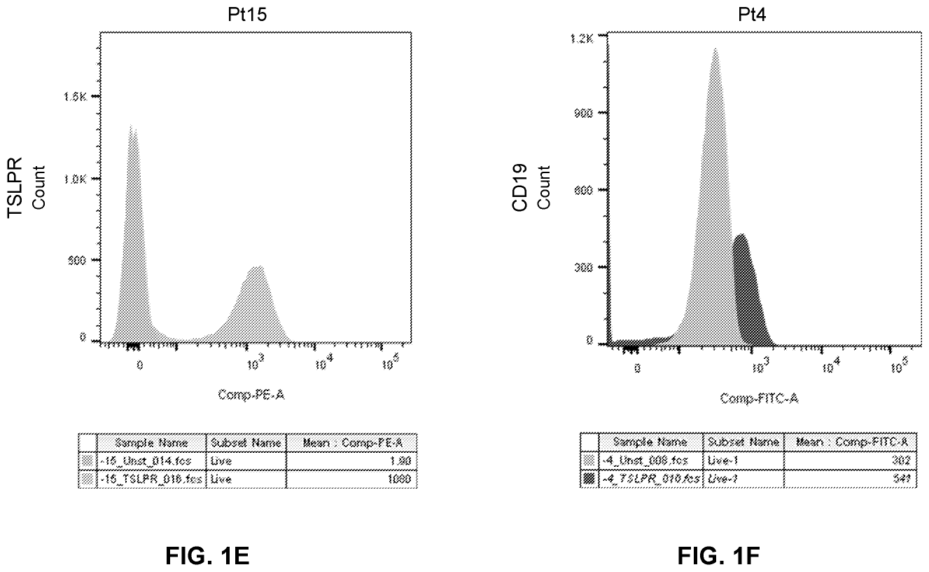

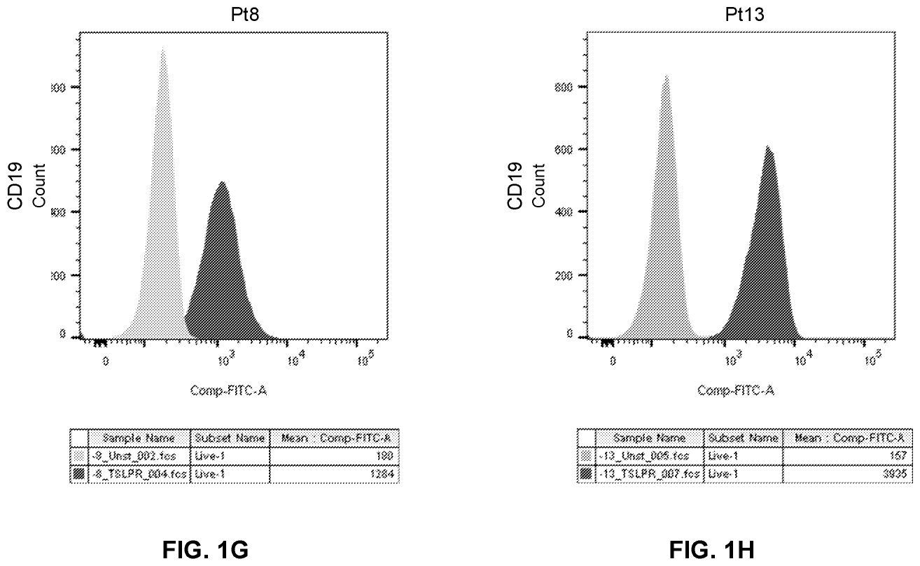

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85, 90, 95 or 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85, 95 to 99% identity to a human germline sequence selected from those listed in FIG. 1 of U.S. Pat. No. 7,657,380.

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain).

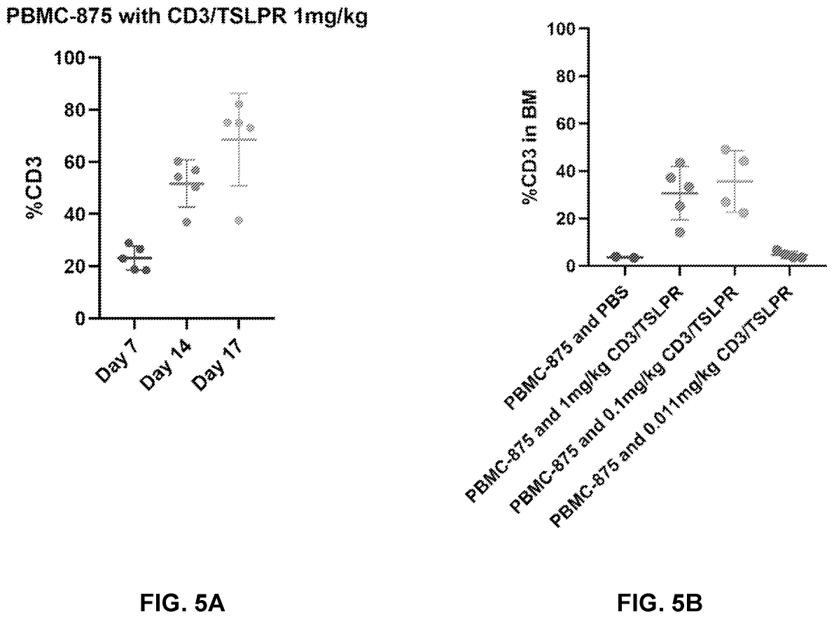

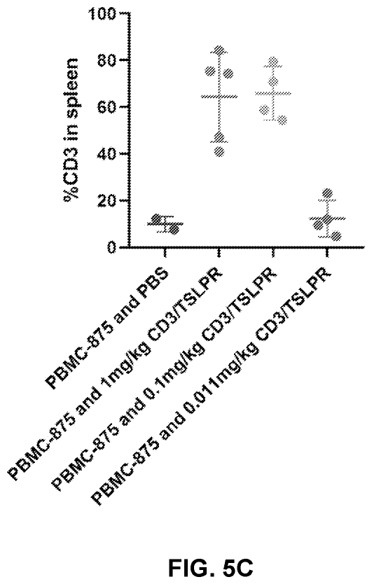

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in FIG. 5.

In some embodiments, the anti-TSLPR x anti-CD3e antibody is a bispecific antibody. In some embodiments, the anti-TSLPR x anti-CD3e antibody is a bivalent antibody. In some embodiments, the anti-TSLPR x anti-CD3e antibody is a trivalent antibody. In some embodiments, the anti-TSLPR x anti-CD3e antibody is a bispecific, bivalent antibody. In some embodiments, the anti-TSLPR x anti-CD3e antibodies include one TSLPR binding domain and one CD3e binding domain. In exemplary embodiments, the anti-TSLPR x anti-CD3e antibody is a bispecific, trivalent antibody. In some embodiments, the anti-TSLPR x anti-CD3e antibodies include one CD3e binding domain and two TSLPR binding domains.

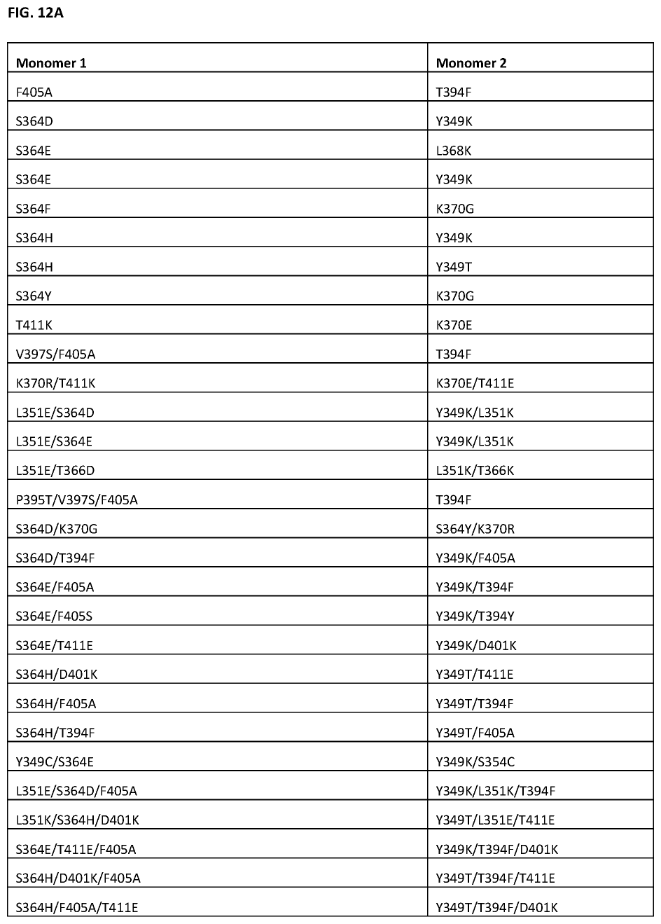

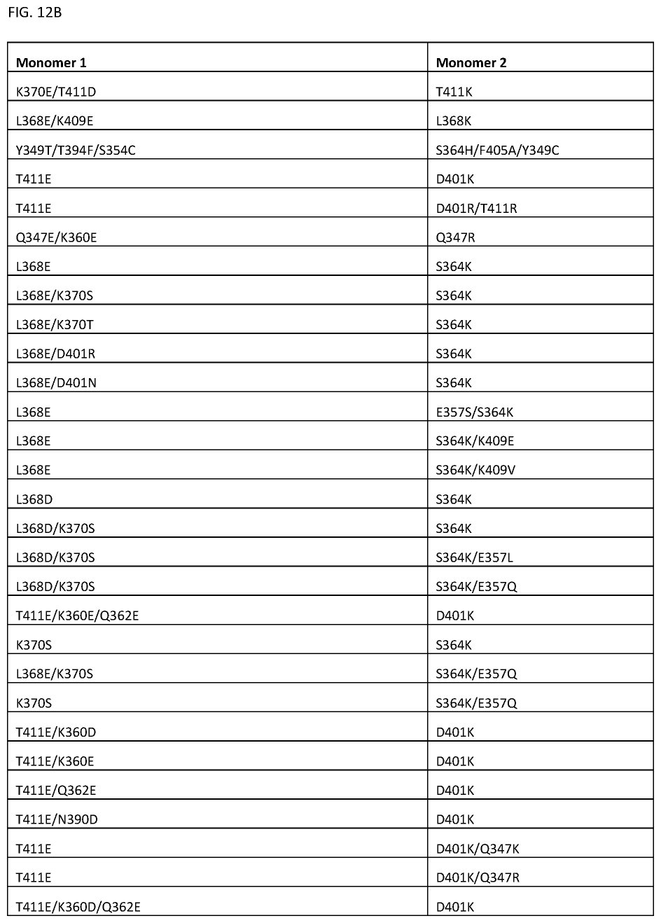

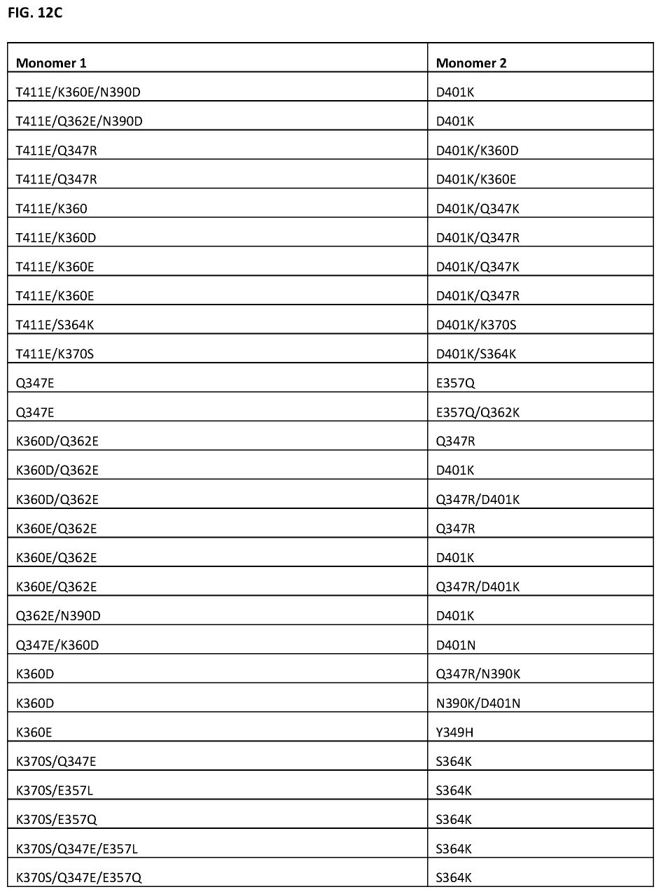

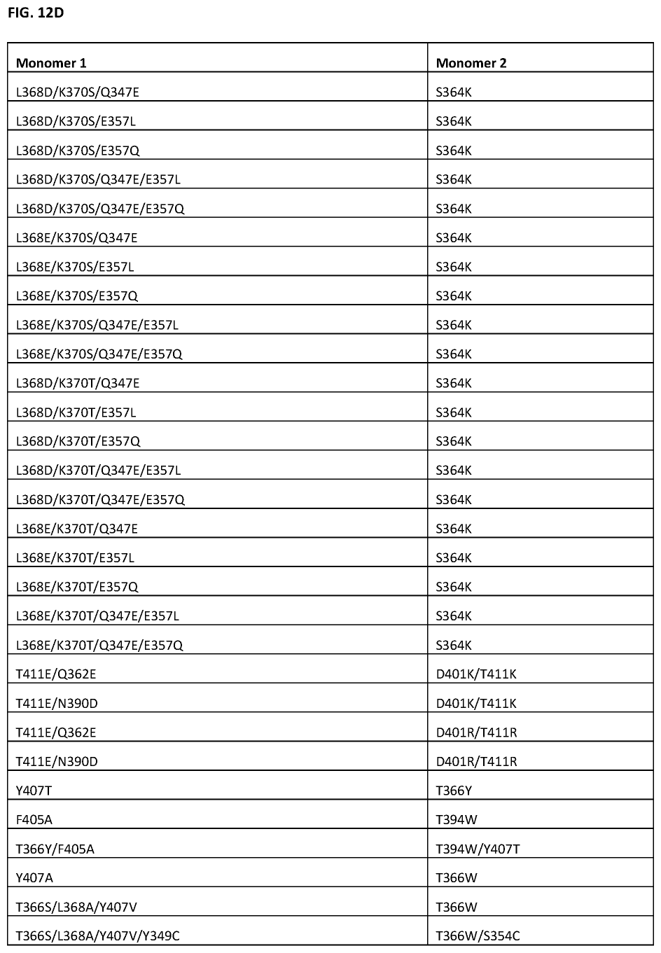

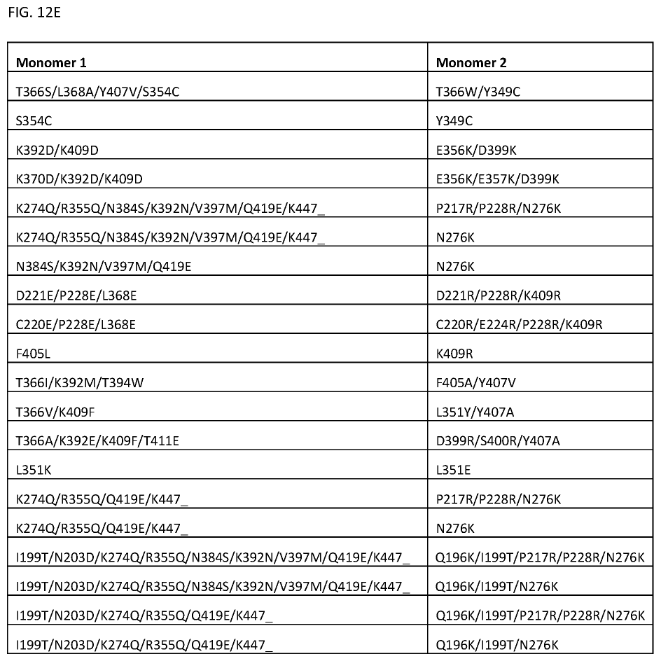

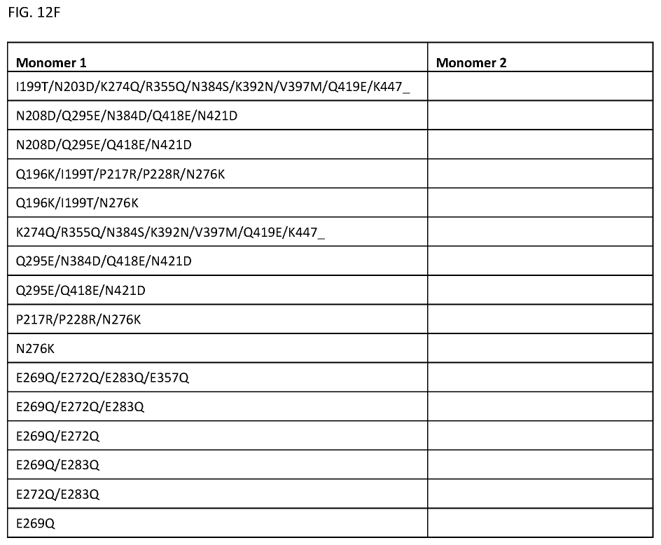

The anti-TSLPR x anti-CD3e antibodies provided herein can be in any useful format, including, including, for example, canonical immunoglobulin, as well as the “1+1 Fab-scFv-Fc,” and “2+1 Fab2-scFv-Fc,” formats described herein (FIG. 23). In some embodiments, the anti-TSLPR x anti-CD3e antibodies are heterodimeric bispecific antibodies that include variant Fc domains having any of the heterodimerization skew variants, pI variants and/or ablation variants described herein. See, e.g. FIGS. 12-14.

Note that unless specified herein, the order of the antigen list in the name does not confer structure; that is an anti-TSLPR x anti-CD3e 1+1 Fab-scFv-Fc antibody can have the scFv bind to CD3e or TSLPR, although in some cases, the order specifies structure as indicated.

The anti-TSLPR x anti-CD3e antibodies provided herein further include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

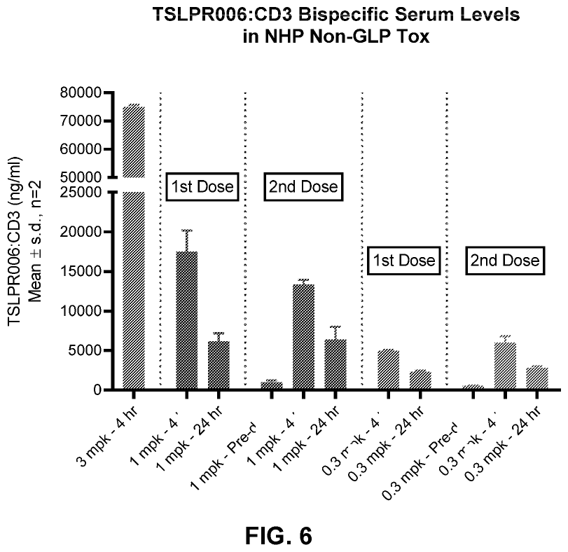

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in FIG. 6. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

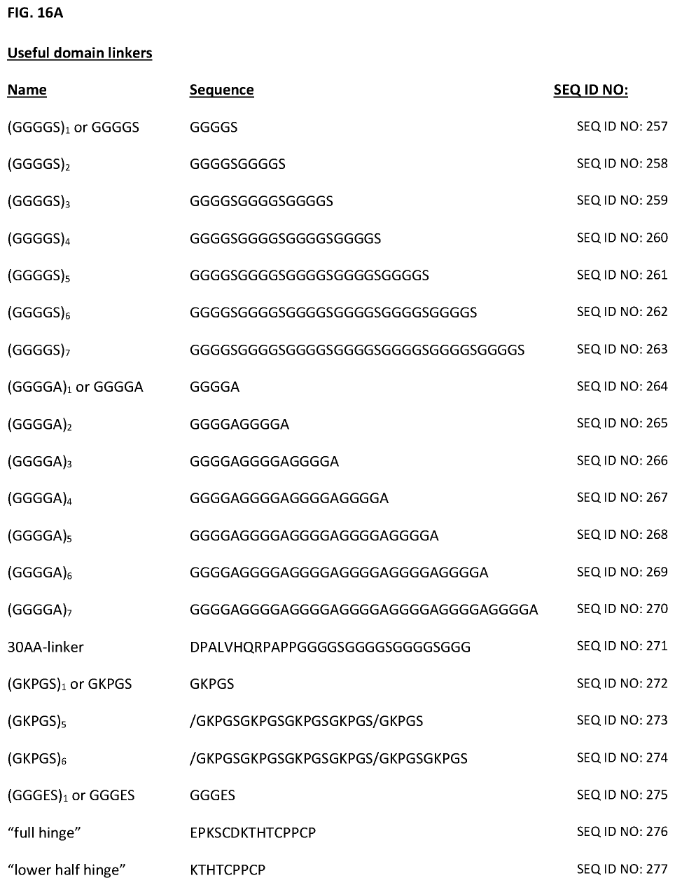

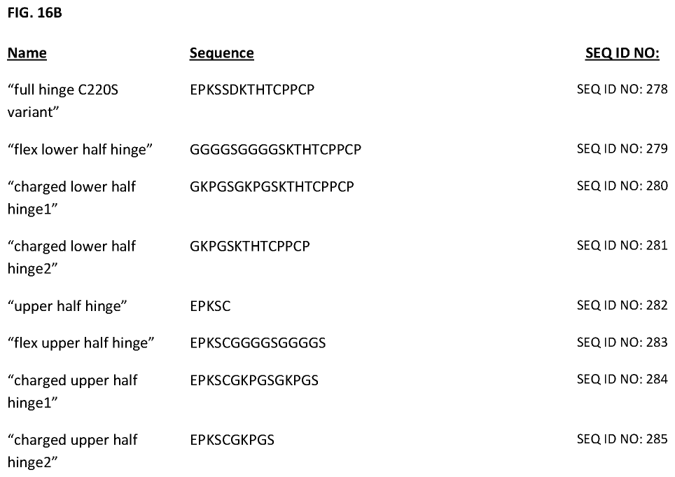

In some embodiments, the linker is a “domain linker”, used to link any two domains as outlined herein together. For example, in the 2+1 Fab2-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 16.

In some embodiments, the linker is a scFv linker that is used to covalently attach the VH and VL domains as discussed herein. In many cases, the scFv linker is a charged scFv linker, a number of which are shown in FIG. 15. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen. Charged domain linkers can also be used to increase the pI separation of the monomers of the invention as well, and thus those included in FIG. 6 can be used in any embodiment herein where a linker is utilized.

Exemplary subject anti-TSLPR x anti-CD3e antibodies are depicted, for example, in FIG. 24. During the cell culture production of the anti-TSLPR x anti-CD3e antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-TSLPR x anti-CD3e antibody is a variant of one of the anti-TSLPR x anti-CD3e antibodies includes a deletion of a C-terminal lysine (−K) terminal or C-terminal lysine and glycine (−GK) residues in one or both Fc domains of an anti-TSLPR x anti-CD3e antibody described herein. In some embodiments, the deletion is G446del and/or K447del (EU numbering).

Aspects of the anti-TSLPR x anti-CD3e antibodies are further described in detail below.

### A. TSLPR Binding Domains

The anti-TSLPR x anti-CD3e antibodies provided herein include at least one TSLPR binding domain that binds to the ECD of human TSLPR. Any suitable TSLPR binding domain can be included in the anti-TSLPR x anti-CD3e antibodies provided herein. In embodiments, the TSLPR binding domain is capable of binding to the extracellular domain of human TSLPR. In embodiments, the TSLPR binding domain is capable of binding to residue 25 to 230 of human TSLPR.

As will be appreciated by those in the art, suitable TSLPR binding domains can comprise a set of 6 CDRs as depicted in Tables 3-10, or identified using other alignments within the variable heavy (VH) domain and variable light domain (VL) sequences of the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165). Suitable TSLPR binding domains can also include the entire VH and VL sequences as depicted in these sequences, used as scFvs or as Fabs. Additional suitable TSLPR binding domains and related vhCDR1-3, vlCDR1-3, and VH/VL sequences that can be used in the subject anti-TSLPR x anti-CD3e antibodies include those disclosed in U.S. Pat. No. 8,475,793 (See, e.g., FIGS. 1-3, Table 2 and Table 3, and hu13H5, h70E8, and h54C11), U.S. Pat. No. 8,097,705 (see, e.g., FIG. 5), and US2012/0020988 (see, e.g., VH domains of SEQ ID NOs: 9 and 16, and VL domains of SEQ ID NOs: 11, 14, 18, and 19), which are all incorporated by reference, particularly in pertinent parts relating to anti-TSLPR antibodies, TSLPR binding domains, and related vhCDR1-3, vlCDR1-3, and VH/VL sequences. In some embodiments, suitable TSLPR binding domains for use in anti-TSLPR x anti-CD3e antibody are capable of binding to residue 25 to 230 of human TSLPR.

In one embodiment, the TSLPR antigen binding domain includes the 6 CDRs (i.e., vhCDR1-3 and vlCDR1-3) of any of the TSLPR binding domains described herein, including, but not limited to the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165). In some embodiments, the TSLPR binding domain includes a VH/VL pair selected from the VHs and VLs of one of the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165).

In addition to the parental CDR sets disclosed in the figures and sequence listing that form an binding domain to TSLPR, provided herein are variant TSLPR binding domains having CDRs that include at least one modification of the TSLPR binding domain CDRs disclosed herein (e.g., (see Tables 3-10 and the sequence listing). In one embodiment, the TSLPR binding domain of the subject anti-TSLPR x anti-CD3e antibody includes a set of 6 CDRs with 1, 2, 3, 4, 5, 6, 7, 8, 9, 10 amino acid modifications as compared to the 6 CDRs of a TSLPR binding domain as described herein, including the figures and sequence listing. In exemplary embodiments, the TSLPR binding domain of the subject anti-TSLPR x anti-CD3e antibody includes a set of 6 CDRs with 1, 2, 3, 4, 5, 6, 7, 8, 9, 10 amino acid modifications as compared to the 6 CDRs of one of the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165). In certain embodiments, the TSLPR binding domain of the subject anti-TSLPR x anti-CD3e antibody is capable of binding TSLPR antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TSLPR binding domain is capable of binding human TSLPR antigen.

In some embodiments, the TSLPR binding domain of the subject anti-TSLPR x anti-CD3e antibody includes 6 CDRs that are at least 90, 95, 97, 98 or 99% identical to the 6 CDRs of a TSLPR binding domain as described herein, including the figures and sequence listing. In exemplary embodiments, the TSLPR binding domain of the subject anti-TSLPR x anti-CD3e antibody includes 6 CDRs that are at least 90, 95, 97, 98 or 99% identical to the 6 CDRs of one of the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165). In certain embodiments, the TSLPR binding domain is capable of binding to the TSLPR, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TSLPR binding domain is capable of binding human TSLPR antigen.

In another exemplary embodiment, the TSLPR binding domain of the subject anti-TSLPR x anti-CD3e antibody includes the variable heavy (VH) domain and variable light (VL) domain of any one of the TSLPR binding domains described herein, including the figures and sequence listing. In exemplary embodiments, the TSLPR binding domain is one of the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165).

In some embodiments, the anti-TSLPR x anti-CD3e antibody includes a TSLPR binding domain that includes a variable heavy domain and/or a variable light domain that are variants of a TSLPR binding domain VH and VL domain disclosed herein. In one embodiment, the variant VH domain and/or VL domain has from 1, 2, 3, 4, 5, 6, 7, 8, 9 or 10 amino acid changes from a VH and/or VL domain of a TSLPR binding domain described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165). In some embodiments, the changes are in a VH domain. In some embodiments, the changes are in a VL domain. In some embodiments, the changes are in a VH and VL domain. In some embodiments, the one or more amino acid changes are in the VH and/or VL framework regions (FR1, FR2, FR3, and/or FR4). In some embodiments, the one or more amino acid change(s) are in one or more CDRs. In certain embodiments, the TSLPR binding domain is capable of binding to TSLPR, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TSLPR binding domain is capable of binding human TSLPR antigen.

In one embodiment, the variant VH and/or VL domain is at least 90, 95, 97, 98 or 99% identical to the VH and/or VL of a TSLPR binding domain as described herein, including the figures and sequence listing. In exemplary embodiments, the variant VH and/or VL domain is at least 90, 95, 97, 98 or 99% identical to the VH and/or VL of one of the following TSLPR binding domains: Clone 1B7 (SEQ ID NOs: 7 and 10), Clone 1C3 (SEQ ID NOs: 8 and 11), Clone 1A9 (SEQ ID NOs: 9 and 12), Clone 2E4 (SEQ ID NOs: 76 and 148), Clone 2H8 (SEQ ID NOs: 77 and 149), Clone 3E5 (SEQ ID NOs: 78 and 150), Clone 1A10 (SEQ ID NOs: 79 and 151), Clone 1B9 (SEQ ID NOs: 80 and 152), Clone 1D10 (SEQ ID NOs: 81 and 153), Clone 1F6 (SEQ ID NOs: 82 and 154), Clone 1G3 (SEQ ID NOs: 83 and 155), Clone 3D8 (SEQ ID NOs: 84 and 156), Clone 10C4 (SEQ ID NOs: 85 and 157), Clone 2A4 (SEQ ID NOs: 86 and 158), Clone 2B2 (SEQ ID NOs: 87 and 159), Clone 2D11 (SEQ ID NOs: 88 and 160), Clone 2E6 (SEQ ID NOs: 89 and 161), Clone 2F1 (SEQ ID NOs: 90 and 162), Clone 2F5 (SEQ ID NOs: 91 and 163), Clone 2H1 (SEQ ID NOs: 92 and 164), and Clone 2H4 (SEQ ID NOs: 93 and 165). In certain embodiments, the TSLPR binding domain is capable of binding to TSLPR, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TSLPR binding domain is capable of binding human TSLPR antigen.

In some embodiments, the anti-TSLPR x anti-CD3e antibody includes a TSLPR binding domain that includes a VH and VL selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:11 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 12 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 148 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 149 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 150 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:151 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 152 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 153 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 155 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 156 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 158 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:160 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 161 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 163 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:164 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof (see, e.g., Tables 3-10).

In some embodiments, the anti-TSLPR x anti-CD3e antibody includes a TSLPR binding domain that includes a VH and VL selected from the following:
(i) a VH having an amino acid sequence of SEQ ID NO:7 or a variant thereof; and (ii) a VL having an amino acid sequence of SEQ ID NO: 10 or a variant thereof; or
(i) a VH having an amino acid sequence of SEQ ID NO:8 or a variant thereof; and (ii) a VL having an amino acid sequence of SEQ ID NO:11 or a variant thereof; or
(i) a VH having an amino acid sequence of SEQ ID NO:9 or a variant thereof; and (ii) a VL having an amino acid sequence of SEQ ID NO:12 or a variant thereof; or
(i) a VH having an amino acid sequence of SEQ ID NO:76 or a variant thereof; and (ii) a VL having an amino acid sequence of SEQ ID NO: 148 or a variant thereof; or
(i) a VH having an amino acid sequence of SEQ ID NO:77 or a variant thereof; and (ii) a VL having an amino acid sequence of SEQ ID NO: 149 or a variant thereof; or
(i) a VH having an amino acid sequence of SEQ ID NO:78 or a variant thereof; and (ii) a VL having an amino acid sequence of SEQ ID NO: 150 or a variant thereof; or
(i) a VH having an amino acid sequence of SEQ ID NO:79 or a variant thereof; and (ii) a VL having an amino acid sequence of SEQ ID NO: 151 or a variant thereof; or
(i) a VH having an amino acid sequence of SEQ ID NO:80 or a variant thereof; and (ii) a VL having an amino acid sequence of SEQ ID NO: 152 or a variant thereof; or
(i) a VH having an amino acid sequence of SEQ ID NO:81 or a variant thereof; and (ii) a VL having an amino acid sequence of SEQ ID NO: 153 or a variant thereof; or
(i) a VH having an amino acid sequence of SEQ ID NO:82 or a variant thereof; and (ii) a VL having an amino acid sequence of SEQ ID NO:154 or a variant thereof; or
(i) a VH having an amino acid sequence of SEQ ID NO:83 or a variant thereof; and (ii) a VL having an amino acid sequence of SEQ ID NO: 155 or a variant thereof; or
(i) a VH having an amino acid sequence of SEQ ID NO:84 or a variant thereof; and (ii) a VL having an amino acid sequence of SEQ ID NO:156 or a variant thereof; or
(i) a VH having an amino acid sequence of SEQ ID NO:85 or a variant thereof; and (ii) a VL having an amino acid sequence of SEQ ID NO:157 or a variant thereof; or
(i) a VH having an amino acid sequence of SEQ ID NO:86 or a variant thereof; and (ii) a VL having an amino acid sequence of SEQ ID NO:158 or a variant thereof; or
(i) a VH having an amino acid sequence of SEQ ID NO:87 or a variant thereof; and (ii) a VL having an amino acid sequence of SEQ ID NO: 159 or a variant thereof; or
(i) a VH having an amino acid sequence of SEQ ID NO:88 or a variant thereof; and (ii) a VL having an amino acid sequence of SEQ ID NO: 160 or a variant thereof; or
(i) a VH having an amino acid sequence of SEQ ID NO:89 or a variant thereof; and (ii) a VL having an amino acid sequence of SEQ ID NO: 161 or a variant thereof; or
(i) a VH having an amino acid sequence of SEQ ID NO:90 or a variant thereof; and (ii) a VL having an amino acid sequence of SEQ ID NO: 162 or a variant thereof; or
(i) a VH having an amino acid sequence of SEQ ID NO:91 or a variant thereof; and (ii) a VL having an amino acid sequence of SEQ ID NO:163 or a variant thereof; or
(i) a VH having an amino acid sequence of SEQ ID NO:92 or a variant thereof; and (ii) a VL having an amino acid sequence of SEQ ID NO:164 or a variant thereof; or
(i) a VH having an amino acid sequence of SEQ ID NO:93 or a variant thereof; and (ii) a VL having an amino acid sequence of SEQ ID NO:165 or a variant thereof.

### B. CD3 Epsilon (CD3e) Binding Domains

The anti-TSLPR x anti-CD3e antibodies provided herein include at least one CD3 epsilon (CD3e) binding domain. Any suitable CD3e binding domain can be included in the anti-TSLPR x anti-CD3e antibodies provided herein. In embodiments, the CD3e binding domain is capable of binding to human CD3e (e.g., N-terminus ECD of human CD3e).

As will be appreciated by those in the art, suitable CD3e binding domains can comprise a set of 6 CDRs as depicted in the sequence listing and FIGS. 24A-E, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 24A-E, and the sequence listing (see Table 2). Suitable CD3e binding domains can also include the entire VH and VL sequences as depicted in these sequences and figures, used as scFvs or as Fab domains. Additional suitable CD3e binding domains and related vhCDR1-3, vlCDR1-3, VH, and VL sequences that can be used in the subject anti-TSLPR x anti-CD3e antibodies include those disclosed in U.S. Pat. No. 7,635,472 (see, e.g., SEQ ID NOs: 65 and 67), U.S. Pat. No. 9,975,949 (see, e.g., SEQ ID NOs: 57 and 58), U.S. Pat. No. 9,657,102 (see, e.g., SEQ ID NOs: 1250, 1266, 1282, 1298, and 1314 (VH), and SEQ ID NOs: 1258, 1274, 1290, 1306, and 1322 (VL)), U.S. Pat. No. 9,969,809 (see, e.g., SEQ ID NOs: 112 and 38), which are all incorporated by reference, particularly in pertinent parts relating to anti-CD3e antibodies, CD3e binding domains, and related vhCDR1-3, vlCDR1-3, and VH/VL sequences. In some embodiments, suitable CD3e binding domains for use in anti-TSLPR x anti-CD3e antibody is capable of binding to the extracellular domain of human CD3e (e.g., amino acids 1-15 of human CD3e).

In one embodiment, the CD3e binding domain of the anti-TSLPR x anti-CD3e antibody includes the 6 CDRs (i.e., vhCDR1-3 and vlCDR1-3) of a CD3e binding domain described herein, including the Figures and sequence listing. In some embodiments, the anti-TSLPR x anti-CD3e antibody includes the 6 CDRs (i.e., vhCDR1-3 and vlCDR1-3) of a CD3e binding domain depicted in FIGS. 24A-E. In exemplary embodiments, the CD3e binding domain is one of the following CD3e binding domains [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E).

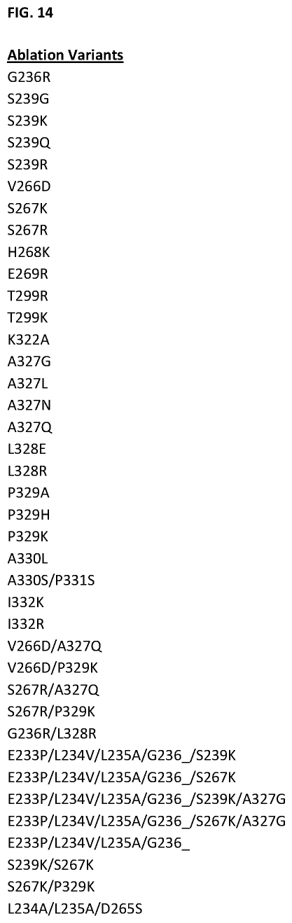

In addition to the parental CDR sets disclosed in the figures and sequence listing that form an binding domain to CD3e, provided herein are variant CD3e binding domains having CDRs that include at least one modification of the CD3e binding domain CDRs disclosed herein. In one embodiment, the CD3e binding domain includes a set of 6 CDRs with 1, 2, 3, 4, 5, 6, 7, 8, 9, 10 amino acid modifications as compared to the 6 CDRs of a CD3e binding domain described herein, including the figures and sequence listing. In exemplary embodiments, the CD3e binding domain of the anti-TSLPR x anti-CD3e antibody includes a set of 6 CDRs with 1, 2, 3, 4, 5, 6, 7, 8, 9, 10 amino acid modifications as compared to the 6 CDRs of one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). In certain embodiments, the variant CD3e binding domain is capable of binding CD3e antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD3e binding domain is capable of binding human CD3e antigen (see FIG. 14).

In one embodiment, the CD3e binding domain of the anti-TSLPR x anti-CD3e antibody includes 6 CDRs that are at least 90, 95, 97, 98 or 99% identical to the 6 CDRs of a CD3e binding domain as described herein, including the figures and sequence listing. In exemplary embodiments, the CD3e binding domain includes 6 CDRs that are at least 90, 95, 97, 98 or 99% identical to the 6 CDRs of one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). In certain embodiments, the CD3e binding domain is capable of binding to CD3e antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD3e binding domain is capable of binding human CD3e antigen (see FIG. 14).

In another exemplary embodiment, the CD3e binding domain of the anti-TSLPR x anti-CD3e antibody include the variable heavy (VH) domain and variable light (VL) domain of any one of the CD3e binding domains described herein, including the figures and sequence listing. In exemplary embodiments, the CD3e binding domain is one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E).

In addition to the parental CD3e variable heavy and variable light domains disclosed herein, provided herein are CD3e binding domains that include a variable heavy domain and/or a variable light domain that are variants of a CD3e binding domain VH and VL domain disclosed herein. In one embodiment, the variant VH domain and/or VL domain has from 1, 2, 3, 4, 5, 6, 7, 8, 9 or 10 amino acid changes from a VH and/or VL domain of a CD3e binding domain described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). In some embodiments, the changes are in a VH domain. In some embodiments, the changes are in a VL domain. In some embodiments, the changes are in a VH and VL domain. In some embodiments, the one or more amino acid changes are in the VH and/or VL framework regions (FR1, FR2, FR3, and/or FR4). In some embodiments, the one or more amino acid change(s) are in one or more CDRs. In certain embodiments, the CD3e binding domain of the anti-TSLPR x anti-CD3e antibody is capable of binding to CD3e, as measured at least one of a Biacore, surface plasmon resonance (SPR), flow cytometry, and/or BLI (biolayer interferometry, e.g., Octet assay) assay, with the latter finding particular use in many embodiments. In particular embodiments, the CD3e binding domain is capable of binding human CD3e antigen.

In one embodiment, the variant VH and/or VL domain is at least 90, 95, 97, 98 or 99% identical to the VH and/or VL of a CD3e binding domain as described herein, including the figures and sequence listing. In exemplary embodiments, the variant VH and/or VL domain is at least 90, 95, 97, 98 or 99% identical to the VH and/or VL of one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). In some embodiments, the CD3e binding domain includes a VH that is at least 90, 95, 97, 98 or 99% identical to the VH domain of one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). In some embodiments, the CD3e binding domain includes a VL that is at least 90, 95, 97, 98 or 99% identical to the VL domain of one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). In some embodiments, the CD3e binding domain includes a VH and a VL that is at least 90, 95, 97, 98 or 99% identical to a VH domain and a VL domain of one of the following CD3e binding domains: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). In certain embodiments, the CD3e binding domain of the anti-TSLPR x anti-CD3e antibody is capable of binding to the CD3e, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD3e binding domain is capable of binding human CD3e antigen.

In some embodiments, the anti-TSLPR x anti-CD3e antibody is a bivalent antibody (e.g., 1+1 Fab-scFv-Fc format antibody) that includes one CD3e binding domain. In other embodiments, the anti-TSLPR x anti-CD3e antibody is a trivalent antibody (e.g., 2+1 mAb-scFv and 2+1 Fab2-scFv-Fc format antibodies) that includes two CD3e binding domains.

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10⁻²⁰ amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272 (16): 10678-10684; Rosok et al., 1996, J. Biol. Chem. 271 (37): 22611-22618; Rader et al., 1998, Proc. Natl. Acad. Sci. USA 95: 8910-8915; Krauss et al., 2003, Protein Engineering 16 (10): 753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Heterodimeric Antibodies

In exemplary embodiments, the anti-TSLPR x anti-CD3e antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

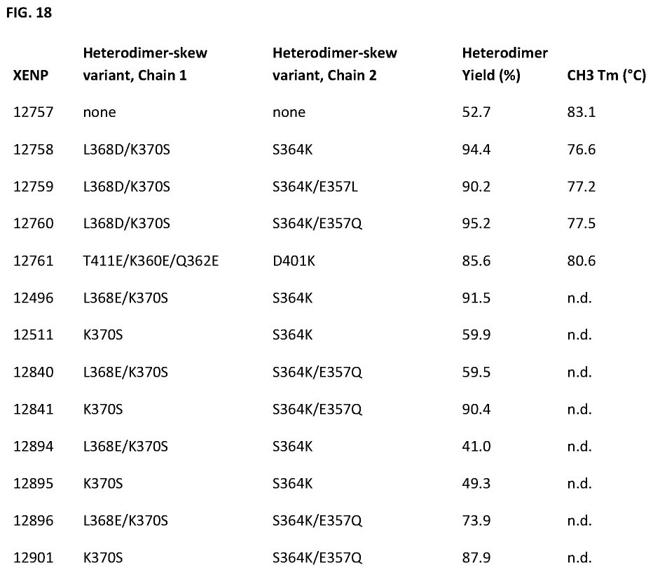

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 12 and 18.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9 (7): 617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285 (25): 19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A: 50% heterodimer A/B: 25% homodimer B/B). Exemplary heterodimerization “skew” variants are depicted in FIG. 1. Such “skew” variants include, but are not limited to: S364K/E357Q: L368D/K370S; L368D/K370S: S364K; L368E/K370S: S364K; T411T/E360E/Q362E: D401K; L368D/K370S: S364K/E357L; K370S: S364K/E357Q (EU numbering).

In exemplary embodiments, the heterodimeric antibody includes a S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C) or T366S/L368A/Y407V/Y354C: T366W/S349C “skew” variant amino acid substitution set (EU numbering). In an exemplary embodiment, the heterodimeric antibody includes a “S364K/E357Q: L368D/K370S” amino acid substitution set. In terms of nomenclature, the pair “S364K/E357Q: L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, Fc heterodimerization domains that result in heterodimerization of two different Fc domains comprise amino acid variants in the Fc domain including, but not limited to, these pairs: S364K/E357Q: L368D/K370S; L368D/K370S: S364K; L368E/K370S: S364K; T411T/E360E/Q362E: D401K; L368D/K370S: S364K/E357L; K370S: S364K/E357Q; or a T366S/L368A/Y407V: T366W (optionally including a bridging disulfide, T366S/L368A/Y407V/Y349C: T366W/S354C or T366S/L368A/Y407V/Y354C: T366W/S349C) “skew” variant amino acid substitution set (EU numbering), as well as others in FIG. 1. In general, these heterodimerization Fc variants are made in human IgG1, IgG2 or IgG4 backbones.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

### 2. Purification Variants

In some embodiments, the heterodimeric antibody includes purification variants that advantageously allow for the separation of heterodimeric proteins (e.g., anti-TSLPR x anti-CD3e bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, and the “2+1 Fab2-scFv-Fc” format, allows separation on the basis of size. As described above, it is also possible to “skew” the formation of heterodimers over homodimers using skew variants. Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab2-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

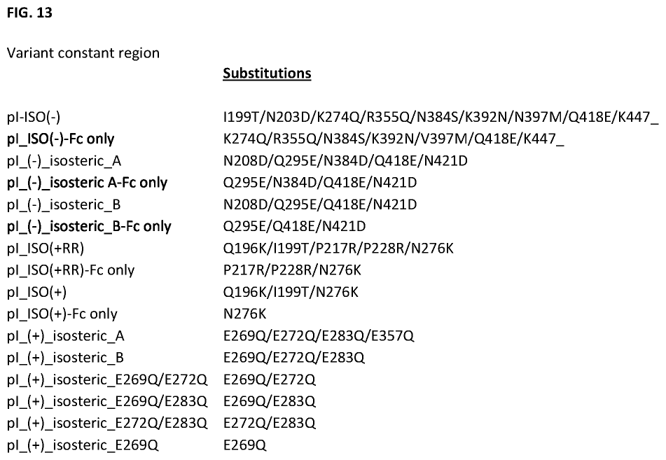

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine). A number of these variants are shown in the FIGS. 12 and 13.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, and 2+1 Fab2-scFv-Fc formats, the starting pI of the scFv (1+1 Fab-scFv-Fc, 2+1 Fab2-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 12 and 13, and FIG. 30 of US Publ. App. No. 2016/0355608, all of which are herein incorporated by reference in its entirety and specifically for the disclosure of pI variants. Preferred combinations of pI variants are shown in FIGS. 12 and 13. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO:238). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

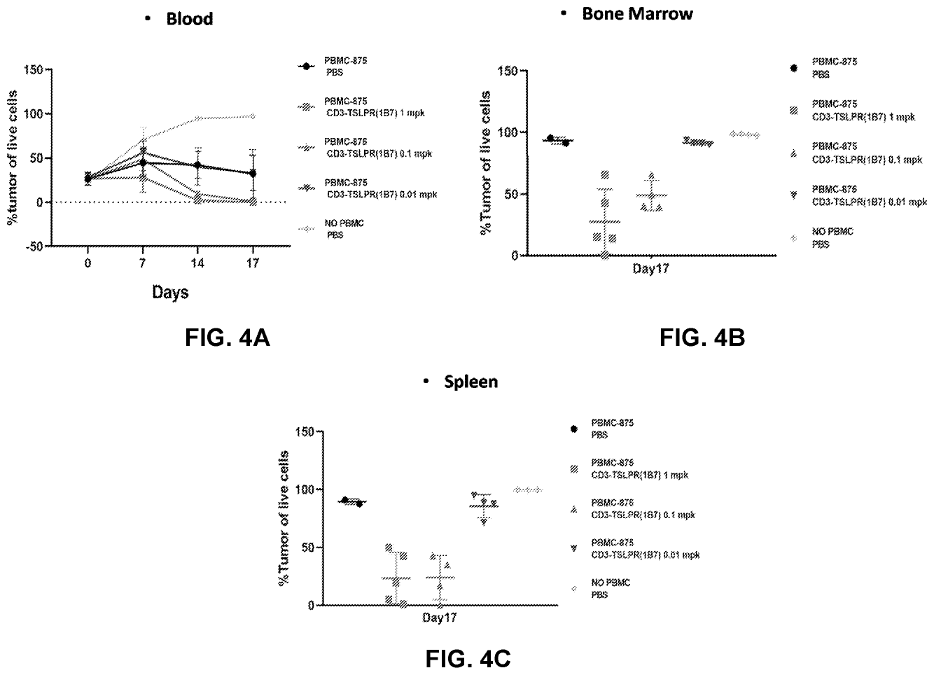

Accordingly, in some embodiments, one monomer has a set of substitutions from FIG. 4 and the other monomer has a charged linker (either in the form of a charged scFv linker because that monomer comprises an scFv or a charged domain linker, as the format dictates, which can be selected from those depicted in FIG. 5).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-TSLPR x anti-CD3e antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). See, e.g., US2010331527, which is incorporated by reference in its entirety, and specifically for pertinent disclosures relating to Fc domain modifications to reduce Protein A binding.

### 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

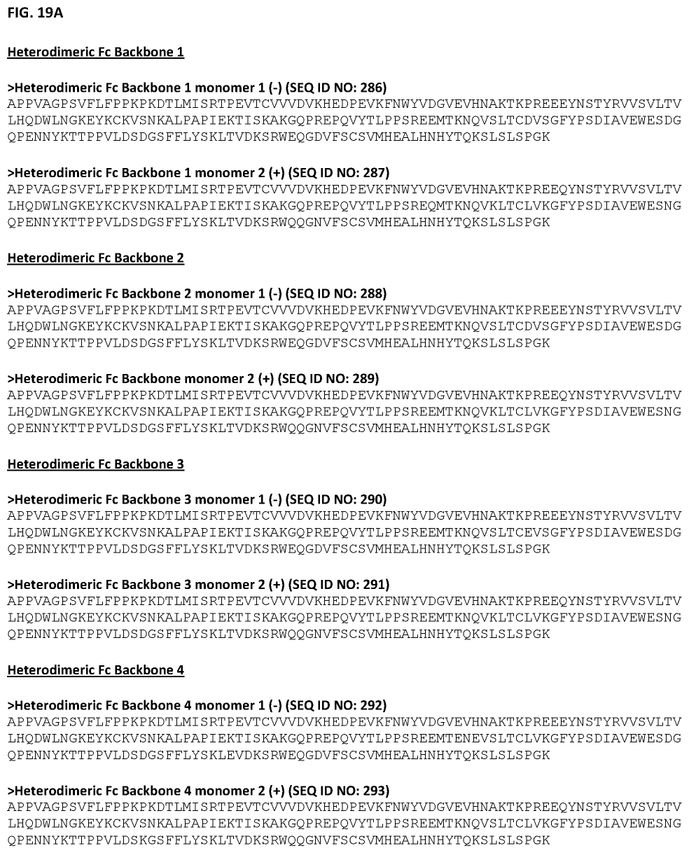

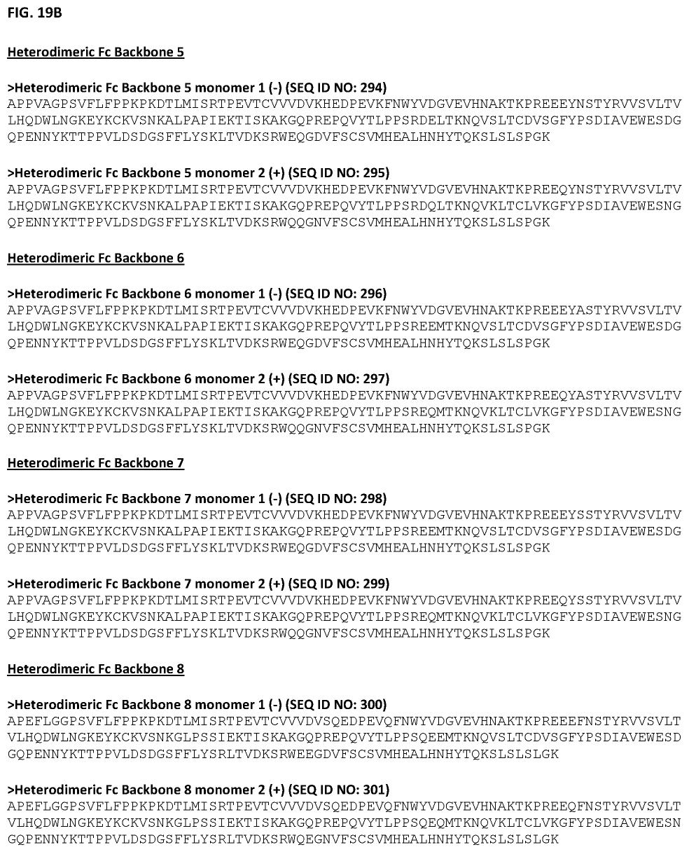

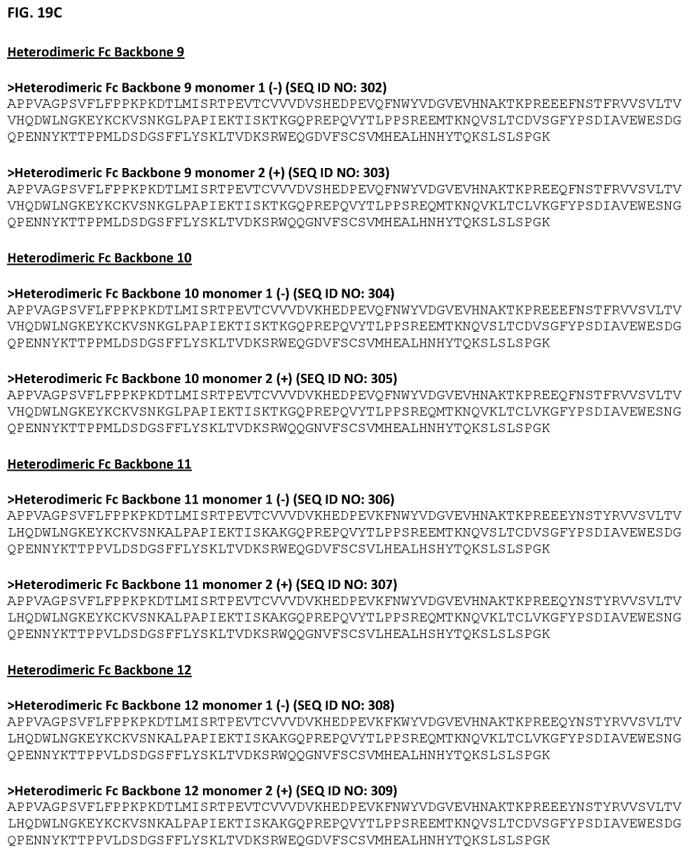

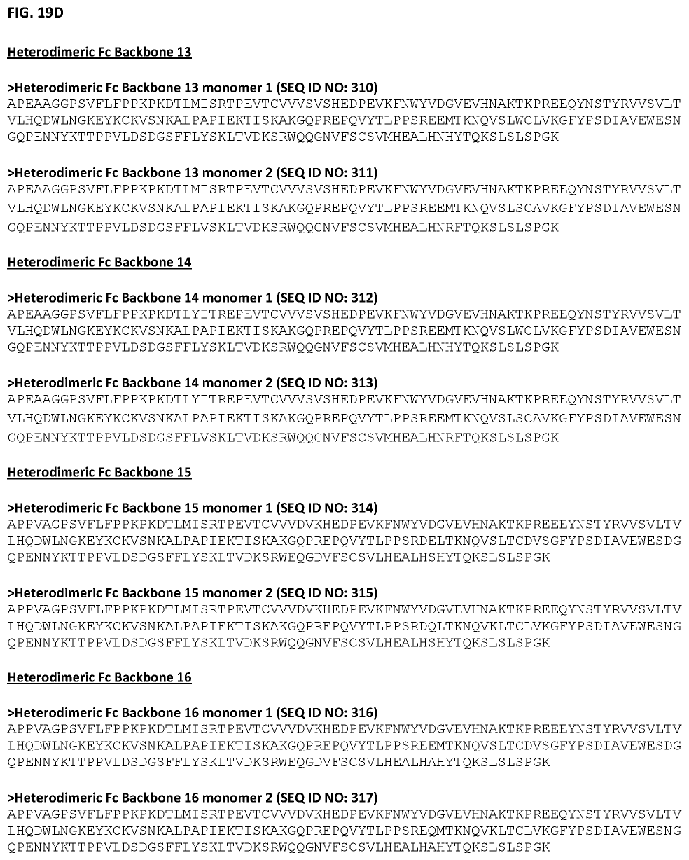

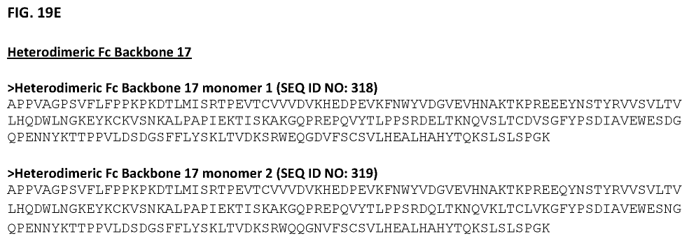

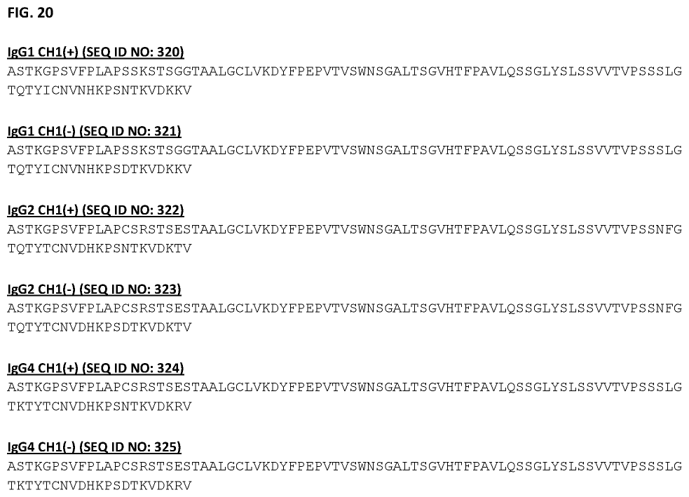

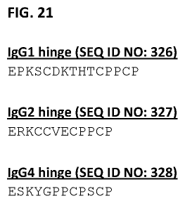

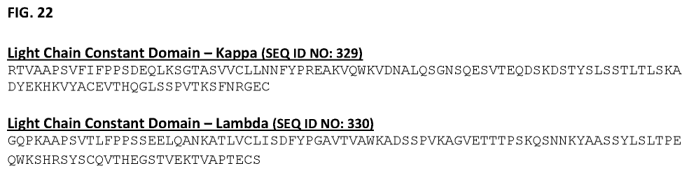

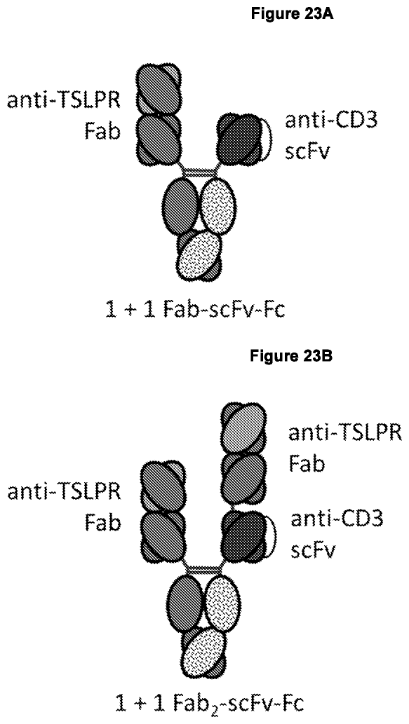

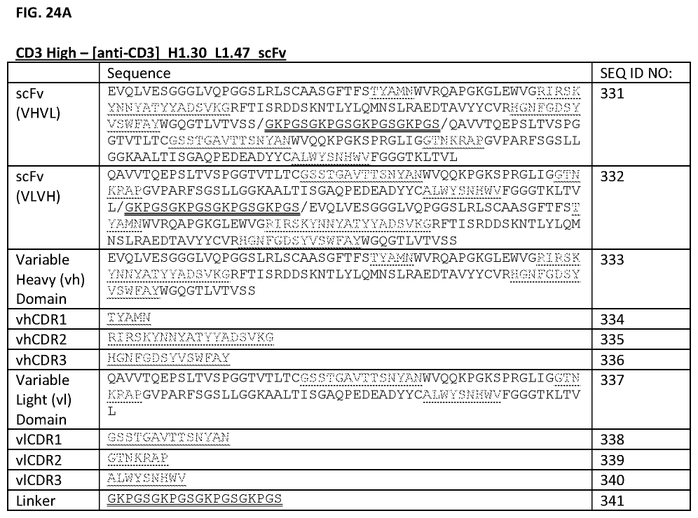

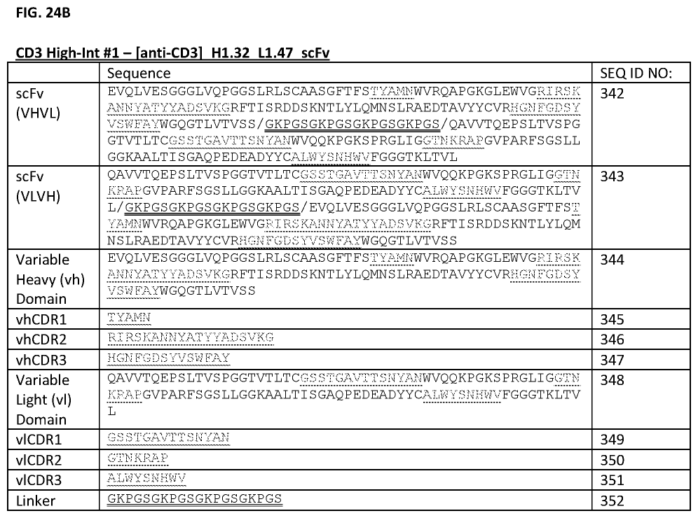

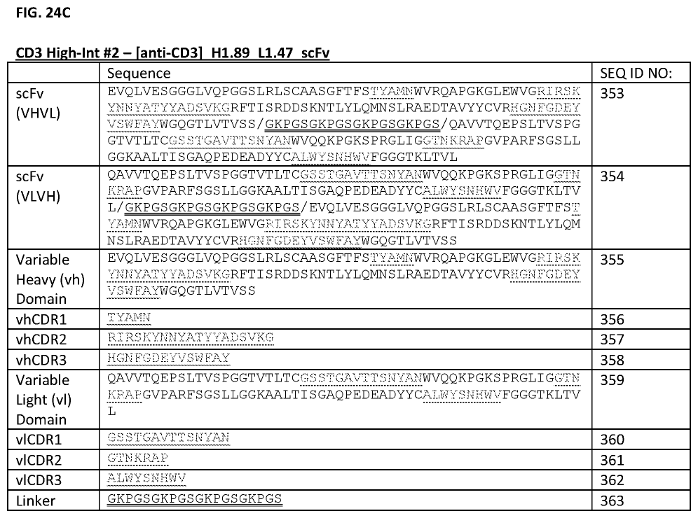

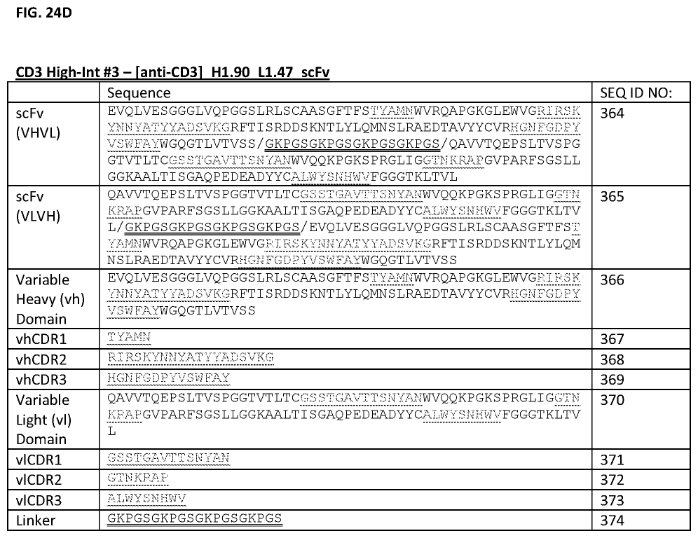

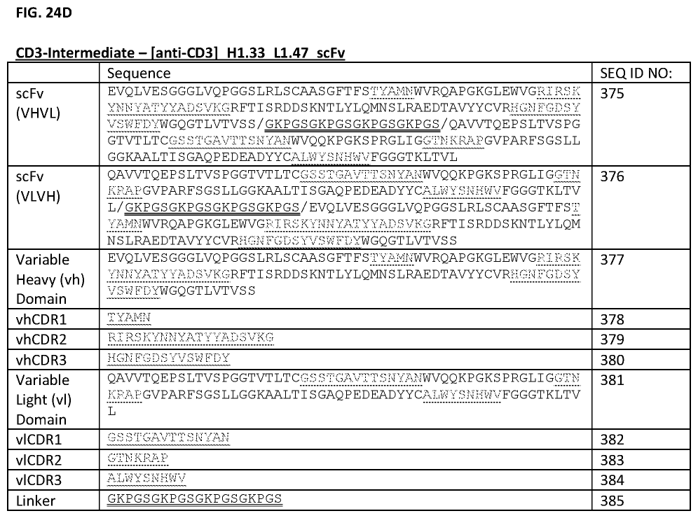

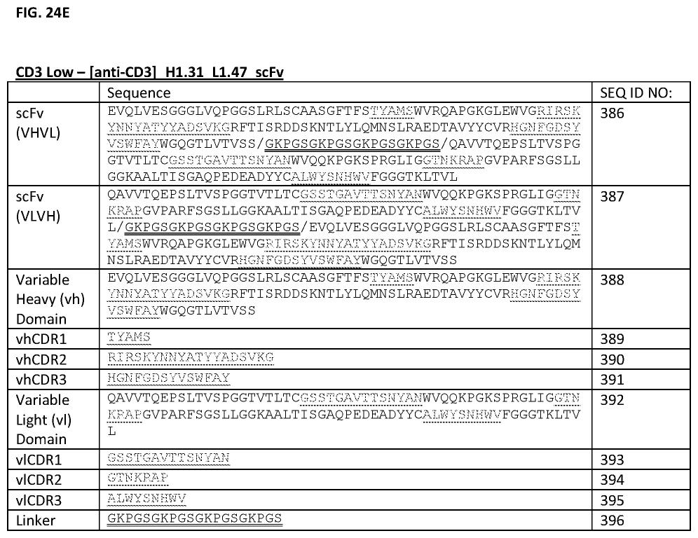

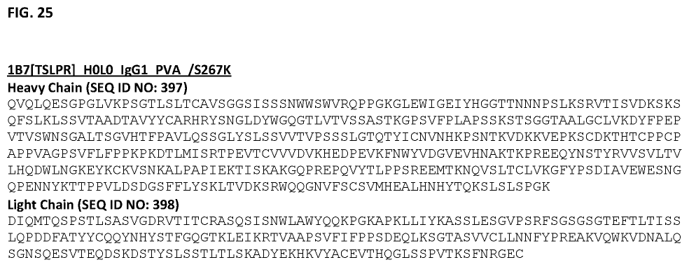

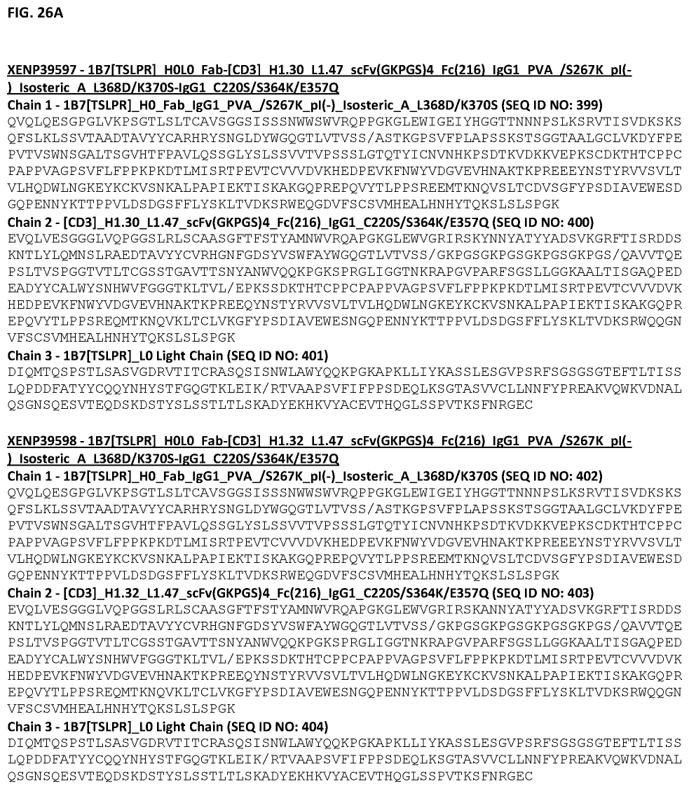

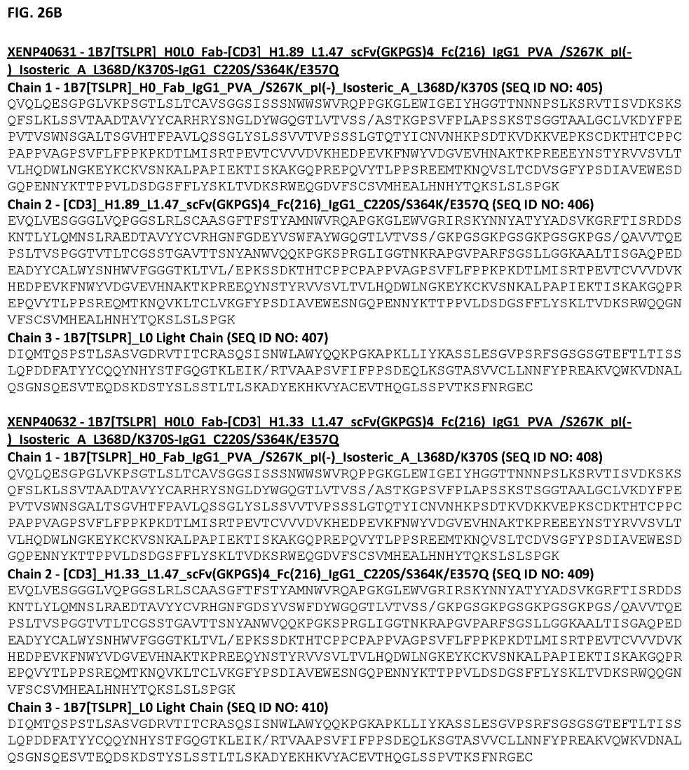

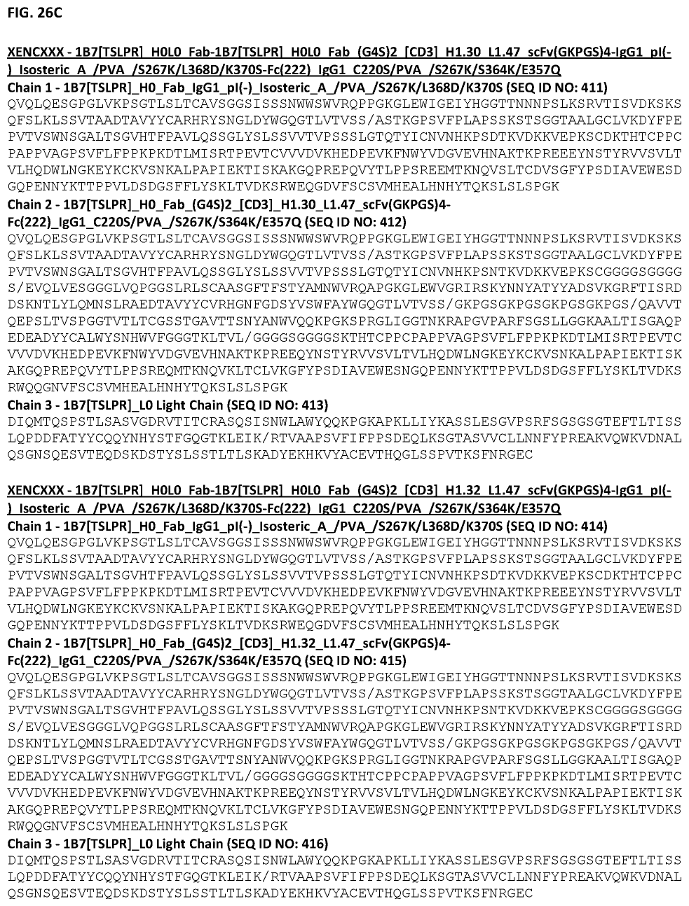

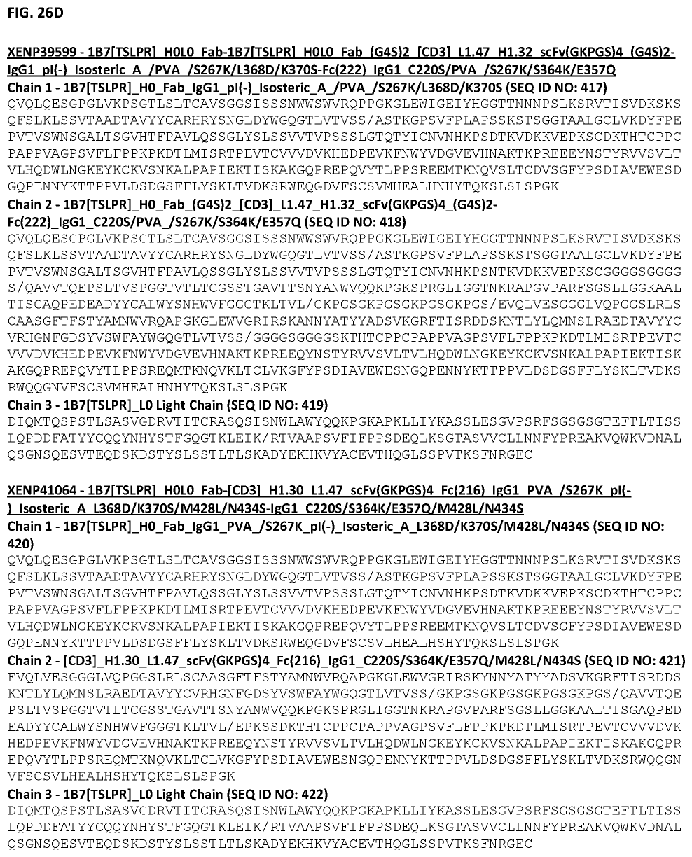

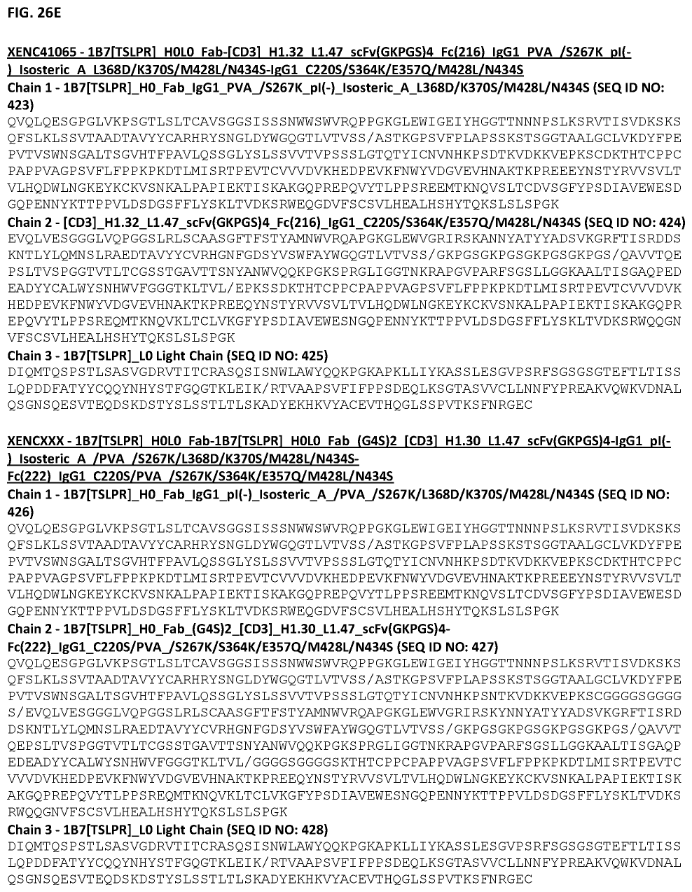

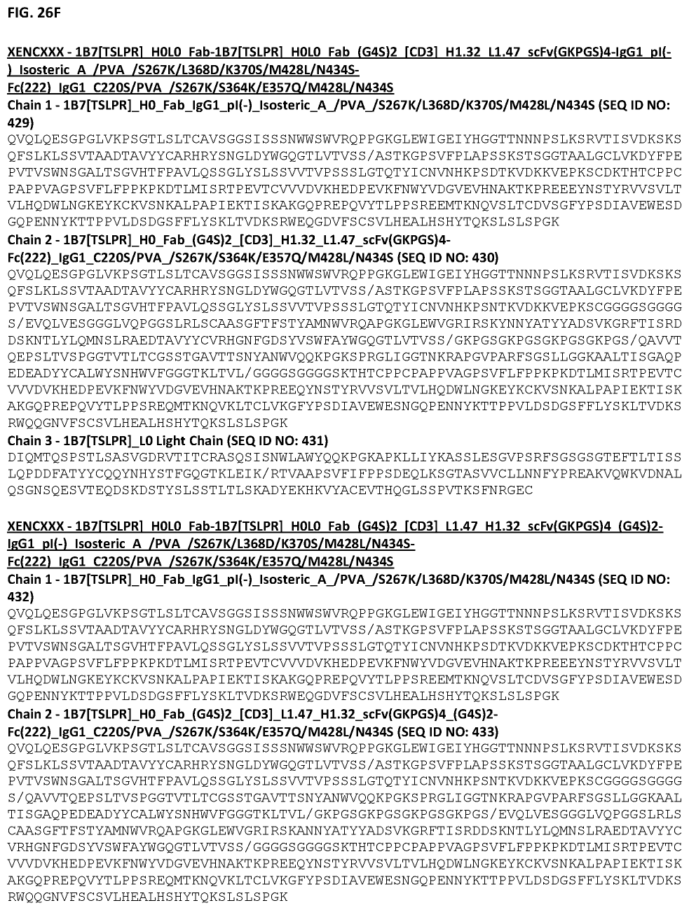

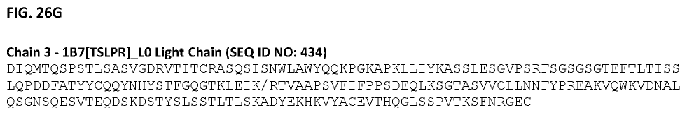

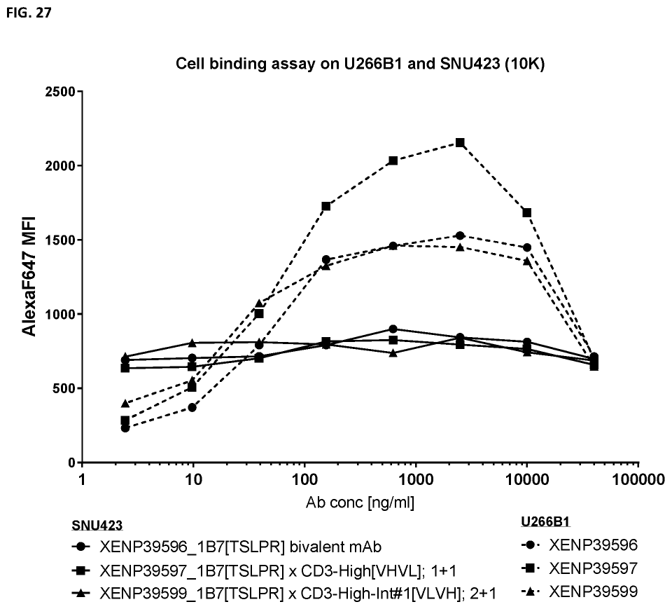

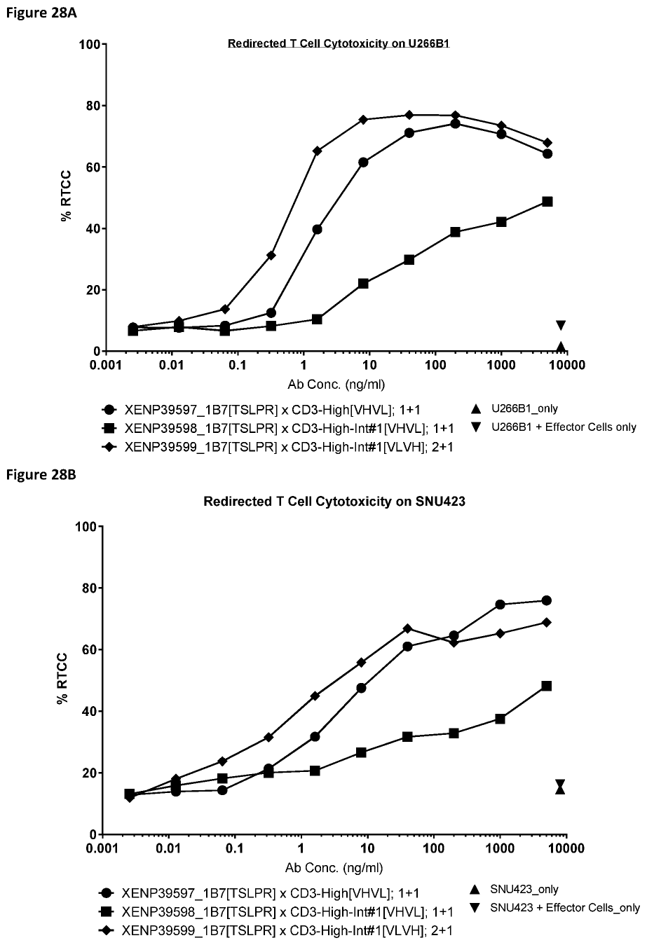

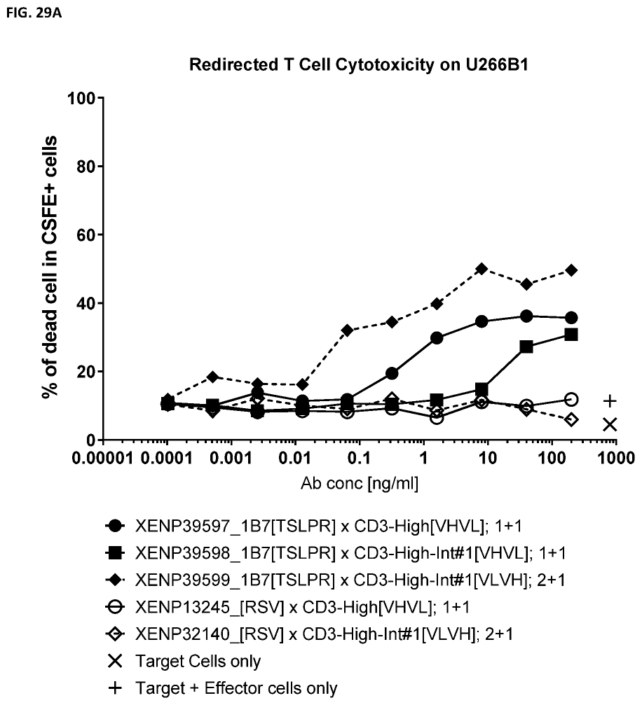

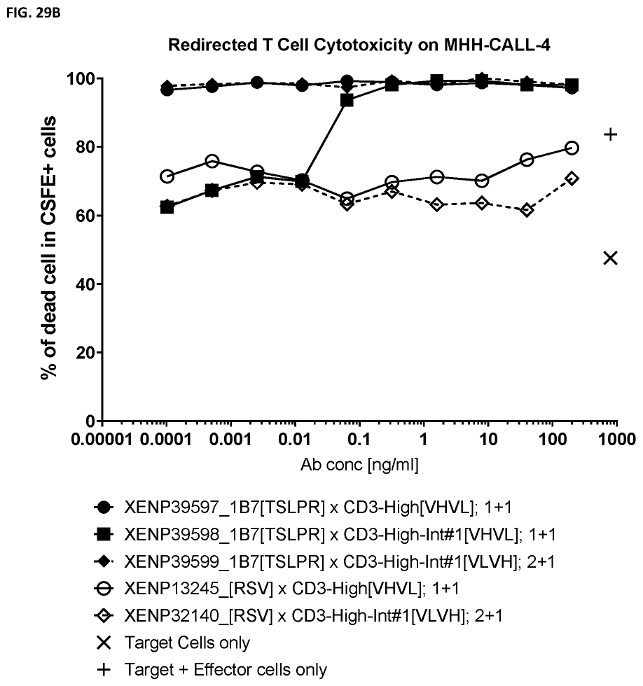

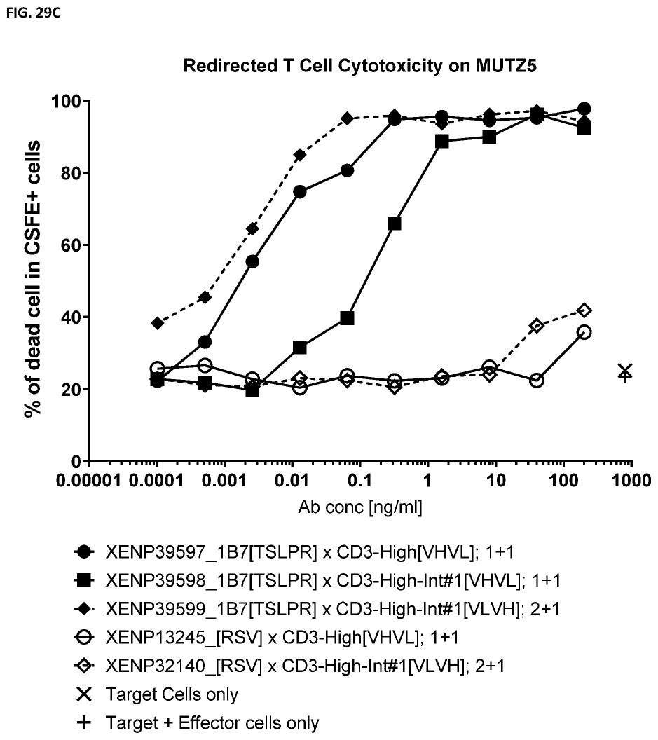

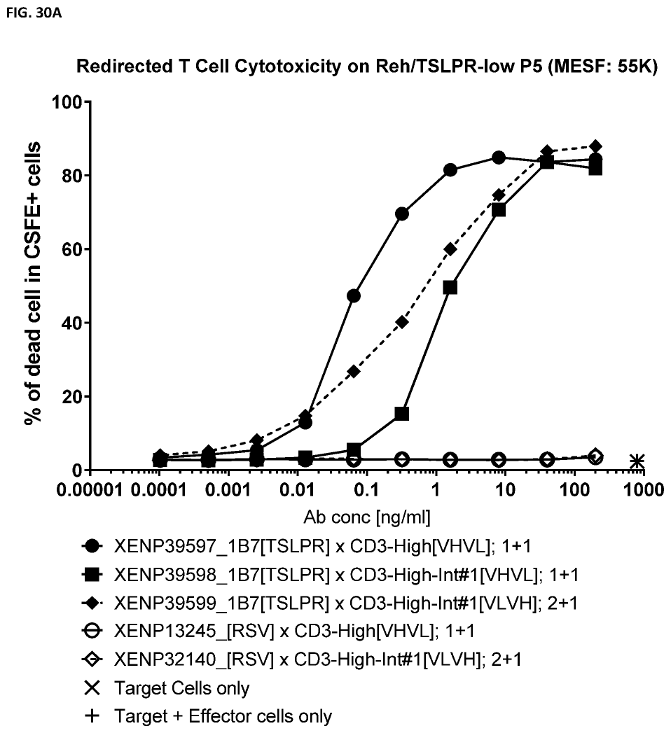

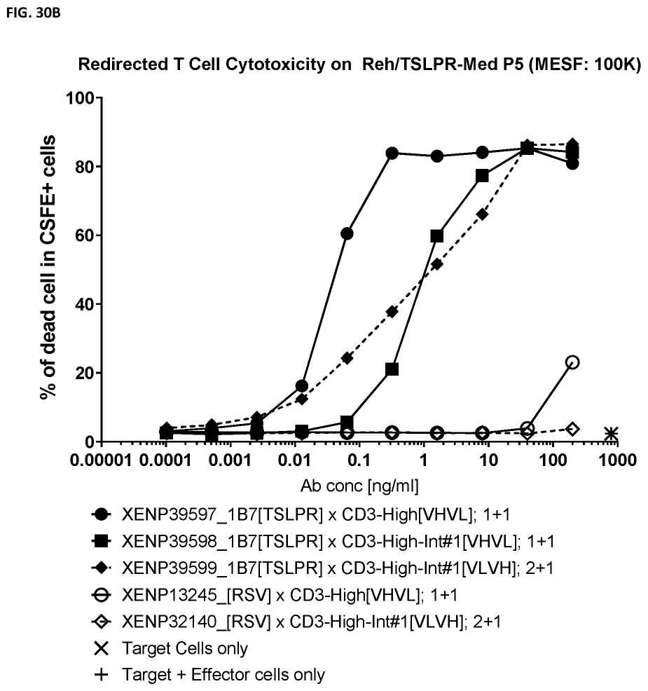

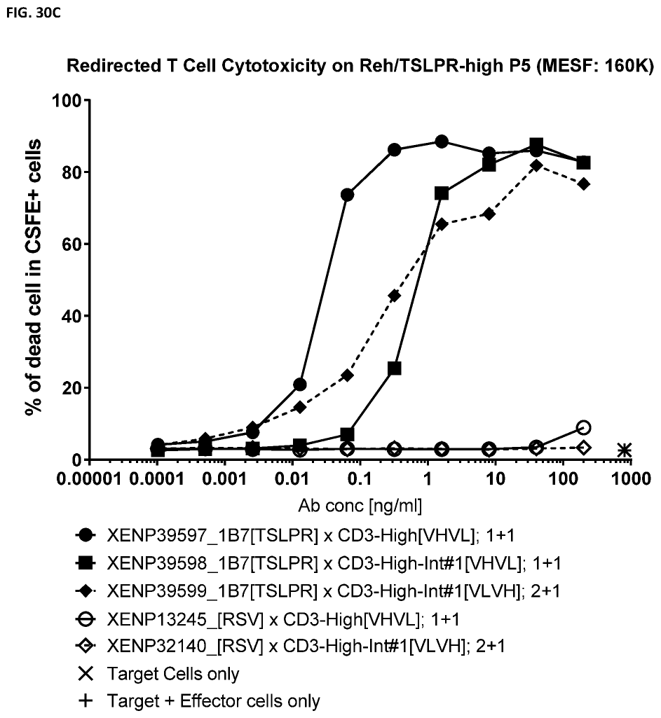

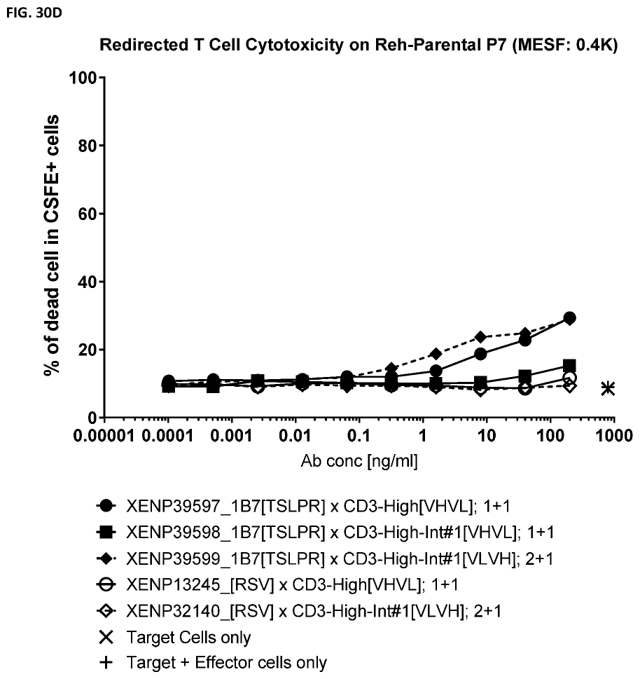

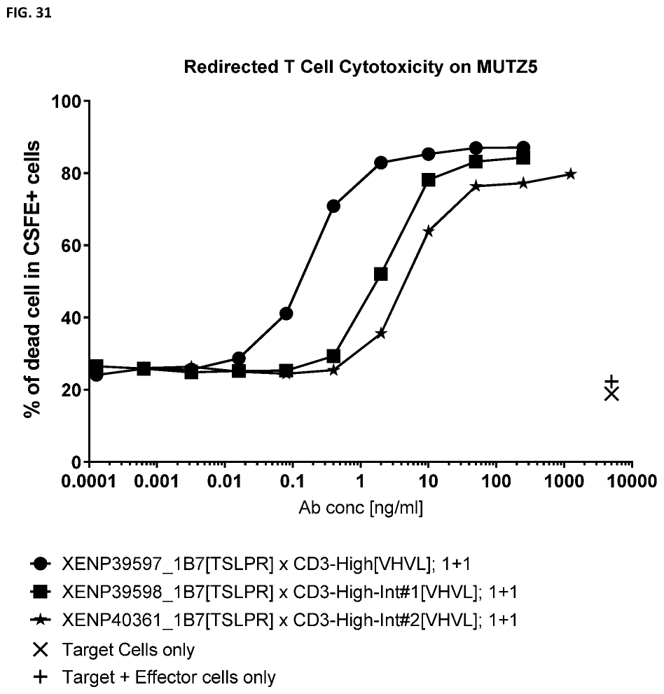

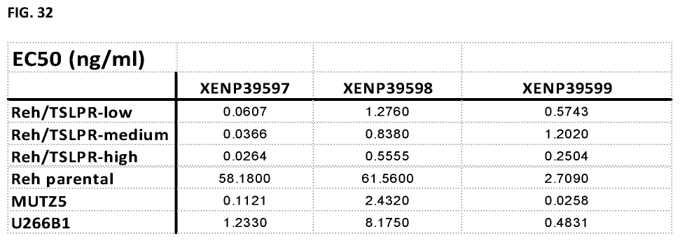

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18 (12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23 (5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. application Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 2591, 428L/434S, 2591/308F, 4361/428L, 4361 or V/434S, 436V/428L, 2591/308F/428L, and M252Y/S254T/T256E. Such modification may be included in one or both Fc domains of the subject antibody.

### 2. Ablation Variants

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD3 epsilon (CD3e) monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 14, and each can be independently and optionally included or excluded, with aspects utilizing ablation variants selected from the group consisting of: L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or purification variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

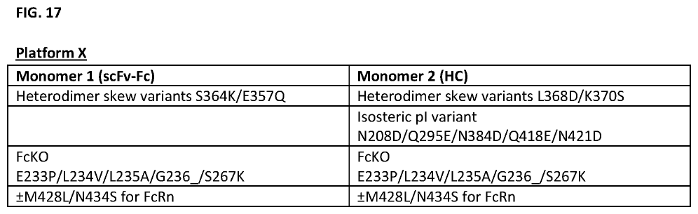

Exemplary combination of variants that are included in some embodiments of the heterodimeric 1+1 Fab-scFv-Fc, and 2+1 Fab2-scFv-Fc format antibodies are included in FIG. 4. In some embodiments, the heterodimeric antibody includes a combination of variants as depicted in FIG. 17.

### G. Useful Antibody Formats

As will be appreciated by those in the art and discussed more fully below, the heterodimeric bispecific antibodies provided herein can take on a wide variety of configurations, as show, for example, in FIG. 23. As will be appreciated by those in the art, the heterodimeric formats of the antibodies described herein can have different valencies as well as be bispecific. That is, heterodimeric antibodies of the antibodies described herein can be bivalent and bispecific, wherein one target tumor antigen (e.g. CD3e) is bound by one binding domain and the other target tumor antigen (e.g. TSLPR is bound by a second binding domain. The heterodimeric antibodies can also be trivalent and bispecific, wherein the first antigen is bound by two binding domains and the second antigen by a second binding domain. In embodiments, it is preferable that the CD3e is bound only monovalently, to reduce potential side effects.

The antibodies described herein utilize anti-CD3e antigen binding domains in combination with anti-CD3e binding domains. As will be appreciated by those in the art, any collection of anti-CD3e CDRs, anti-CD3 variable light and variable heavy domains, Fabs and scFvs as depicted in any of the Figures can be used (see, e.g., FIG. 24). Similarly, any of the TSLPR binding domains can be used, whether CDRs, variable light and variable heavy domains, Fabs and scFvs as disclosed herein can be used, optionally and independently combined in any combination.

### 1. 1+1 Fab-scFv-Fc Format

One heterodimeric antibody format that finds particular use in subject anti-TSLPR x anti-CD3e antibodies provided herein is the “1+1 Fab-scFv-Fc” or “bottle opener” format as shown in FIG. 23A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv”, as defined below) and a second Fc domain. The scFv includes a second variable heavy domain (VH2) and a second variable light domain (VL2), wherein the VH2 is attached to the VL2 using an scFv linker that can be charged (see, e.g., FIG. 15). The scFv is attached to the heavy chain using a domain linker (see, e.g., FIG. 16). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a TSLPR binding domain and the other binding domain is a CD3 epsilon (CD3e) binding domain. In some embodiments where the 1+1 Fab-scFv-Fc, it is the scFv that binds to the CD3e, and the Fab that binds TSLPR. Exemplary anti-TSLPR x anti-CD3e bispecific antibodies in the 1+1 Fab-scFv-Fc format are depicted in FIG. 26.

In some embodiments, the first and second Fc domains of the 1+1 Fab-scFv-Fc format antibody are variant Fc domains that include heterodimerization skew variants (e.g., a set of amino acid substitutions as shown in FIGS. 12 and 18). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 14). In some embodiments, each of the first and second variant Fc domains include ablation variants E233P/L234V/L235A/G236/S267K, wherein numbering is according to EU numbering.

In some embodiments, the constant domain (CH1-hinge-CH2-CH3) of the first monomer includes pI variants (including those shown in FIG. 13). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody includes a combination of amino acid modifications as depicted in FIG. 17. In such embodiments, the CH1-hinge-CH2-CH3 of the first monomer comprises amino acid variants L368D/K370S/N208D/Q295E/N384D/Q418E/N421D/E233P/L234V/L235A/G236del/S267K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 15). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody, the first Fc domain includes heterodimerization skew variants L368D/K370S and the second Fc domain includes heterodimerization skew variants S364K/E357Q; each of the first and second Fc domains include ablation variants E233P/L234V/L235A/G236/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker. In some embodiments, the 1+1 Fab-scFv-Fc format antibody provided herein includes FcRn variants M428L/N434S, wherein numbering is according to EU numbering. In some embodiments, the scFv of the 1+1 Fab-scFv-Fc format antibody provided herein includes a charged scFv linker (including those shown in FIG. 15).

Any suitable CD3e binding domain can be included in subject 1+1 Fab-scFv-Fc format antibody, including any of the CD3e binding domains provided herein. In some embodiments, the CD3e binding domain is one of the following CD3e binding domains or a variant thereof: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). Additional suitable CD3e binding domains and related vhCDR1-3, vlCDR1-3, VH, and VL sequences that can be used in the subject anti-TSLPR x anti-CD3e 1+1 Fab-scFv-Fc format antibodies include those disclosed in U.S. Pat. No. 7,635,472 (see, e.g., SEQ ID NOs: 65 and 67), U.S. Pat. No. 9,975,949 (see, e.g., SEQ ID NOs: 57 and 58), U.S. Pat. No. 9,657,102 (see, e.g., SEQ ID NOs: 1250, 1266, 1282, 1298, and 1314 (VH), and SEQ ID NOs: 1258, 1274, 1290, 1306, and 1322 (VL)), U.S. Pat. No. 9,969,809 (see, e.g., SEQ ID NOs: 112 and 38), which are all incorporated by reference, particularly in pertinent parts relating to anti-CD3e antibodies, CD3e binding domains, and related vhCDR1-3, vlCDR1-3, and VH/VL sequences.

In some embodiments of the 1+1 Fab-scFv-Fc format, the CD3e binding domain has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

In some embodiments of the 1+1 Fab-scFv-Fc format, the CD3e binding domain has a VH and VL domain selected from the following:
(i) a VH having an amino acid sequence of SEQ ID NO:333 or a variant thereof; and (ii) a VL having an amino acid sequence of SEQ ID NO:337 or a variant thereof; or
(i) a VH having an amino acid sequence of SEQ ID NO:344 or a variant thereof; and (ii) a VL having an amino acid sequence of SEQ ID NO:348 or a variant thereof; or
(i) a VH having an amino acid sequence of SEQ ID NO:355 or a variant thereof; and (ii) a VL having an amino acid sequence of SEQ ID NO:359 or a variant thereof; or
(i) a VH having an amino acid sequence of SEQ ID NO:366 or a variant thereof; and (ii) a VL having an amino acid sequence of SEQ ID NO:370 or a variant thereof; or
(i) a VH having an amino acid sequence of SEQ ID NO:377 or a variant thereof; and (ii) a VL having an amino acid sequence of SEQ ID NO:381 or a variant thereof; or
(i) a VH having an amino acid sequence of SEQ ID NO:388 or a variant thereof; and (ii) a VL having an amino acid sequence of SEQ ID NO:392 or a variant thereof.

Any suitable TSLPR binding domain can be included in subject 1+1 Fab-scFv-Fc format antibody, including any of the TSLPR binding domains provided herein. In some embodiments, the TSLPR binding domain is one of the following TSLPR binding domains or a variant thereof: Clone 1B7, Clone 1C3, Clone 1A9, Clone 2E4, Clone 2H8, Clone 3E5, Clone 1A10, Clone 1B9, Clone 1D10, Clone 1F6, Clone 1G3, Clone 3D8, Clone 10C4, Clone 2A4, Clone 2B2, Clone 2D11, Clone 2E6, Clone 2F1, Clone 2F5, Clone 2H1, and Clone 2H4. Additional suitable TSLPR binding domains and related vhCDR1-3, vlCDR1-3, and VH/VL sequences that can be used in the subject anti-TSLPR x anti-CD3e 1+1 Fab-scFv-Fc format antibody include those disclosed in U.S. Pat. No. 8,475,793 (See, e.g., FIGS. 1-3, Table 2 and Table 3, and hu13H5, h70E8, and h54C11), U.S. Pat. No. 8,097,705 (see, e.g., FIG. 5), and US2012/0020988 (see, e.g., VH domains of SEQ ID NOs: 9 and 16, and VL domains of SEQ ID NOs: 11, 14, 18, and 19), which are all incorporated by reference, particularly in pertinent parts relating to anti-TSLPR antibodies, TSLPR binding domains, and related vhCDR1-3, vlCDR1-3, and VH/VL sequences.

In some embodiments of the 1+1 Fab-scFv-Fc format, the TSLPR binding domain has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:11 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:12 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:148 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 149 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 78 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 150 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:151 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 152 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 153 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 155 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:156 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 158 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:159 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 160 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:161 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:162 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:163 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:164 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof.

In some embodiments of the 1+1 Fab-scFv-Fc format, the TSLPR binding domain has a VH and VL domain selected from the following:
(i) a VH having an amino acid sequence of SEQ ID NO:7 or a variant thereof; and (ii) a VL having an amino acid sequence of SEQ ID NO: 10 or a variant thereof; or
(i) a VH having an amino acid sequence of SEQ ID NO:8 or a variant thereof; and (ii) a VL having an amino acid sequence of SEQ ID NO:11 or a variant thereof; or
(i) a VH having an amino acid sequence of SEQ ID NO:9 or a variant thereof; and (ii) a VL having an amino acid sequence of SEQ ID NO:12 or a variant thereof; or
(i) a VH having an amino acid sequence of SEQ ID NO:76 or a variant thereof; and (ii) a VL having an amino acid sequence of SEQ ID NO: 148 or a variant thereof; or
(i) a VH having an amino acid sequence of SEQ ID NO:77 or a variant thereof; and (ii) a VL having an amino acid sequence of SEQ ID NO: 149 or a variant thereof; or
(i) a VH having an amino acid sequence of SEQ ID NO:78 or a variant thereof; and (ii) a VL having an amino acid sequence of SEQ ID NO:150 or a variant thereof; or
(i) a VH having an amino acid sequence of SEQ ID NO:79 or a variant thereof; and (ii) a VL having an amino acid sequence of SEQ ID NO:151 or a variant thereof; or
(i) a VH having an amino acid sequence of SEQ ID NO:80 or a variant thereof; and (ii) a VL having an amino acid sequence of SEQ ID NO: 152 or a variant thereof; or
(i) a VH having an amino acid sequence of SEQ ID NO:81 or a variant thereof; and (ii) a VL having an amino acid sequence of SEQ ID NO:153 or a variant thereof; or
(i) a VH having an amino acid sequence of SEQ ID NO:82 or a variant thereof; and (ii) a VL having an amino acid sequence of SEQ ID NO:154 or a variant thereof; or
(i) a VH having an amino acid sequence of SEQ ID NO:83 or a variant thereof; and (ii) a VL having an amino acid sequence of SEQ ID NO:155 or a variant thereof; or
(i) a VH having an amino acid sequence of SEQ ID NO:84 or a variant thereof; and (ii) a VL having an amino acid sequence of SEQ ID NO: 156 or a variant thereof; or
(i) a VH having an amino acid sequence of SEQ ID NO:85 or a variant thereof; and (ii) a VL having an amino acid sequence of SEQ ID NO: 157 or a variant thereof; or
(i) a VH having an amino acid sequence of SEQ ID NO:86 or a variant thereof; and (ii) a VL having an amino acid sequence of SEQ ID NO:158 or a variant thereof; or
(i) a VH having an amino acid sequence of SEQ ID NO:87 or a variant thereof; and (ii) a VL having an amino acid sequence of SEQ ID NO: 159 or a variant thereof; or
(i) a VH having an amino acid sequence of SEQ ID NO:88 or a variant thereof, and (ii) a VL having an amino acid sequence of SEQ ID NO: 160 or a variant thereof; or
(i) a VH having an amino acid sequence of SEQ ID NO:89 or a variant thereof; and (ii) a VL having an amino acid sequence of SEQ ID NO:161 or a variant thereof; or
(i) a VH having an amino acid sequence of SEQ ID NO:90 or a variant thereof; and (ii) a VL having an amino acid sequence of SEQ ID NO: 162 or a variant thereof; or
(i) a VH having an amino acid sequence of SEQ ID NO:91 or a variant thereof; and (ii) a VL having an amino acid sequence of SEQ ID NO:163 or a variant thereof; or
(i) a VH having an amino acid sequence of SEQ ID NO:92 or a variant thereof; and (ii) a VL having an amino acid sequence of SEQ ID NO:164 or a variant thereof; or
(i) a VH having an amino acid sequence of SEQ ID NO:93 or a variant thereof; and (ii) a VL having an amino acid sequence of SEQ ID NO:165 or a variant thereof.

FIG. 19 shows some exemplary Fc domain sequences that are useful in the 1+1 Fab-scFv-Fc format antibodies. The “monomer 1” sequences depicted in FIG. 19 typically refer to the Fc domain of the “Fab-Fc heavy chain” and the “monomer 2” sequences refer to the Fc domain of the “scFv-Fc heavy chain.” In addition, FIGS. 20 and 21 provide exemplary CH1-hinge domains, CH1 domains, and hinge domains that can be included in the first or second monomer of the 1+1 Fab-scFv-Fc format. Further, FIG. 22 provides useful CL sequences that can be used with this format.

### 2. 2+1 Fab2-scFv-Fc Format

One heterodimeric antibody format that finds particular use in subject anti-TSLPR x anti-CD3e antibodies provided herein is the 2+1 Fab2-scFv-Fc format (also referred to as “central-scFv format”) shown in FIG. 23B. This antibody format includes three antigen binding domains: two Fab portions and an scFv that is inserted between the VH-CH1 and CH2-CH3 regions of one of the monomers. In some embodiments of this format, the Fab portions each bind TSLPR and the “extra” scFv domain binds CD3e. In some embodiments, the 2+1 Fab2-scFv-Fc format antibody is a trivalent antibody.

In some embodiments of the 2+1 Fab2-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 16. This embodiment further utilizes a common light chain that includes a variable light domain (VL1) and a constant light domain (CL). The common light chain associates with the VH1-CH1 of the first and second monomers to form two identical Fabs. In some embodiments, the identical Fabs each bind TSLPR. As for many of the embodiments herein, these constructs can include skew variants, pI variants, ablation variants, additional Fc variants, etc. as desired and described herein.

In some embodiments, the first and second Fc domains of the 2+1 Fab2-scFv-Fc format antibody are variant Fc domains that include heterodimerization skew variants (e.g., a set of amino acid substitutions as shown in FIGS. 12 and 18). Particularly useful heterodimerization skew variants include S364K/E357Q: L368D/K370S; L368D/K370S: S364K; L368E/K370S: S364K; T411T/E360E/Q362E: D401K; L368D/K370S: S364K/E357L; K370S: S364K/E357Q; T366S/L368A/Y407V: T366W and T366S/L368A/Y407V/Y349C: T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 14). In some embodiments, each of the first and second variant Fc domains include ablation variants E233P/L234V/L235A/G236/S267K, wherein numbering is according to EU numbering.

In some embodiments, the constant domain (CH1-hinge-CH2-CH3) of the first monomer includes pI variants (including those shown in FIG. 13). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab2-scFv-Fc format antibody provided herein includes a charged scFv linker (including those shown in FIG. 15). In some embodiments, the 2+1 Fab2-scFv-Fc format antibody provided herein includes FcRn variants M428L/N434S, wherein numbering is according to EU numbering.

In exemplary embodiments of the 2+1 Fab2-scFv-Fc format antibody, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker. In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-scFv-Fc format antibody is a CD3 epsilon (CD3e) binding and the VH1 of the first and second monomer and the VL1 of the common light chain each form a binding domain that binds TSLPR. Any suitable CD3e binding domain can be included in subject 2+1 Fab2-scFv-Fc format antibody, including any of the CD3e binding domains provided herein. In some embodiments, the CD3e binding domain is one of the following CD3e binding domains or a variant thereof: [anti-CD3]_H1.30_L1.47, [anti-CD3]_H1.32_L1.47, [anti-CD3]_H1.89_L1.47, [anti-CD3]_H1.90_L1.47, [anti-CD3]_H1.33_L1.47, and [anti-CD3]_H1.31_L1.47 (FIGS. 24A-E). Additional suitable CD3e binding domains and related vhCDR1-3, vlCDR1-3, VH, and VL sequences that can be used in the subject anti-TSLPR x anti-CD3e 2+1 Fab2-scFv-Fc format antibodies include those disclosed in U.S. Pat. No. 7,635,472 (see, e.g., SEQ ID NOs: 65 and 67), U.S. Pat. No. 9,975,949 (see, e.g., SEQ ID NOs: 57 and 58), U.S. Pat. No. 9,657,102 (see, e.g., SEQ ID NOs: 1250, 1266, 1282, 1298, and 1314 (VH), and SEQ ID NOs: 1258, 1274, 1290, 1306, and 1322 (VL)), U.S. Pat. No. 9,969,809 (see, e.g., SEQ ID NOs: 112 and 38), which are all incorporated by reference, particularly in pertinent parts relating to anti-CD3e antibodies, CD3e binding domains, and related vhCDR1-3, vlCDR1-3, and VH/VL sequences.
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

In some embodiments of the 2+1 Fab2-scFv-Fc format, the CD3e binding domain has a VH and VL domain selected from the following:
(i) a VH having an amino acid sequence of SEQ ID NO:333 or a variant thereof; and (ii) a VL having an amino acid sequence of SEQ ID NO:337 or a variant thereof; or
(i) a VH having an amino acid sequence of SEQ ID NO:344 or a variant thereof; and (ii) a VL having an amino acid sequence of SEQ ID NO:348 or a variant thereof; or
(i) a VH having an amino acid sequence of SEQ ID NO:355 or a variant thereof; and (ii) a VL having an amino acid sequence of SEQ ID NO:359 or a variant thereof; or
(i) a VH having an amino acid sequence of SEQ ID NO:366 or a variant thereof; and (ii) a VL having an amino acid sequence of SEQ ID NO:370 or a variant thereof; or
(i) a VH having an amino acid sequence of SEQ ID NO:377 or a variant thereof; and (ii) a VL having an amino acid sequence of SEQ ID NO:381 or a variant thereof; or
(i) a VH having an amino acid sequence of SEQ ID NO:388 or a variant thereof; and (ii) a VL having an amino acid sequence of SEQ ID NO:392 or a variant thereof.

In some embodiments, the VH1 of the first and second monomer and the VL1 of the common light chain of the 2+1 Fab2-scFv-Fc format antibody each form a binding domain that binds TSLPR. In some embodiments, the TSLPR binding domain is one of the following TSLPR binding domains or a variant thereof: Clone 1B7, Clone 1C3, Clone 1A9, Clone 2E4, Clone 2H8, Clone 3E5, Clone 1A10, Clone 1B9, Clone 1D10, Clone 1F6, Clone 1G3, Clone 3D8, Clone 10C4, Clone 2A4, Clone 2B2, Clone 2D11, Clone 2E6, Clone 2F1, Clone 2F5, Clone 2H1, and Clone 2H4. Additional suitable TSLPR binding domains and related vhCDR1-3, vlCDR1-3, and VH/VL sequences that can be used in the subject anti-TSLPR x anti-CD3e 2+1 Fab2-scFv-Fc format antibody include those disclosed in U.S. Pat. No. 8,475,793 (See, e.g., FIGS. 1-3, Table 2 and Table 3, and hu13H5, h70E8, and h54C11), U.S. Pat. No. 8,097,705 (see, e.g., FIG. 5), and US2012/0020988 (see, e.g., VH domains of SEQ ID NOs: 9 and 16, and VL domains of SEQ ID NOs: 11, 14, 18, and 19), which are all incorporated by reference, particularly in pertinent parts relating to anti-TSLPR antibodies, TSLPR binding domains, and related vhCDR1-3, vlCDR1-3, and VH/VL sequences.

In some embodiments of 2+1 Fab2-scFv-Fc format, the TSLPR binding domains each include a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:11 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 12 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 148 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 149 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 150 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO: 79 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:151 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 152 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 153 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:154 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:155 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 156 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 158 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 160 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:161 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 163 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:164 or a variant thereof; or
(i) a VH comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof.

In some embodiments of 2+1 Fab2-scFv-Fc format, the TSLPR binding domains each include a VH and VL domain selected from the following:
(i) a VH having an amino acid sequence of SEQ ID NO:7 or a variant thereof; and (ii) a VL having an amino acid sequence of SEQ ID NO: 10 or a variant thereof; or
(i) a VH having an amino acid sequence of SEQ ID NO:8 or a variant thereof; and (ii) a VL having an amino acid sequence of SEQ ID NO:11 or a variant thereof; or
(i) a VH having an amino acid sequence of SEQ ID NO:9 or a variant thereof; and (ii) a VL having an amino acid sequence of SEQ ID NO:12 or a variant thereof; or
(i) a VH having an amino acid sequence of SEQ ID NO:76 or a variant thereof; and (ii) a VL having an amino acid sequence of SEQ ID NO: 148 or a variant thereof; or
(i) a VH having an amino acid sequence of SEQ ID NO:77 or a variant thereof; and (ii) a VL having an amino acid sequence of SEQ ID NO: 149 or a variant thereof; or
(i) a VH having an amino acid sequence of SEQ ID NO:78 or a variant thereof; and (ii) a VL having an amino acid sequence of SEQ ID NO:150 or a variant thereof; or
(i) a VH having an amino acid sequence of SEQ ID NO:79 or a variant thereof; and (ii) a VL having an amino acid sequence of SEQ ID NO:151 or a variant thereof; or
(i) a VH having an amino acid sequence of SEQ ID NO:80 or a variant thereof; and (ii) a VL having an amino acid sequence of SEQ ID NO:152 or a variant thereof; or
(i) a VH having an amino acid sequence of SEQ ID NO:81 or a variant thereof; and (ii) a VL having an amino acid sequence of SEQ ID NO:153 or a variant thereof; or
(i) a VH having an amino acid sequence of SEQ ID NO:82 or a variant thereof; and (ii) a VL having an amino acid sequence of SEQ ID NO:154 or a variant thereof; or
(i) a VH having an amino acid sequence of SEQ ID NO:83 or a variant thereof; and (ii) a VL having an amino acid sequence of SEQ ID NO: 155 or a variant thereof; or
(i) a VH having an amino acid sequence of SEQ ID NO:84 or a variant thereof; and (ii) a VL having an amino acid sequence of SEQ ID NO:156 or a variant thereof; or
(i) a VH having an amino acid sequence of SEQ ID NO:85 or a variant thereof; and (ii) a VL having an amino acid sequence of SEQ ID NO: 157 or a variant thereof; or
(i) a VH having an amino acid sequence of SEQ ID NO:86 or a variant thereof; and (ii) a VL having an amino acid sequence of SEQ ID NO:158 or a variant thereof; or
(i) a VH having an amino acid sequence of SEQ ID NO:87 or a variant thereof; and (ii) a VL having an amino acid sequence of SEQ ID NO: 159 or a variant thereof; or
(i) a VH having an amino acid sequence of SEQ ID NO:88 or a variant thereof; and (ii) a VL having an amino acid sequence of SEQ ID NO: 160 or a variant thereof; or
(i) a VH having an amino acid sequence of SEQ ID NO:89 or a variant thereof; and (ii) a VL having an amino acid sequence of SEQ ID NO:161 or a variant thereof; or
(i) a VH having an amino acid sequence of SEQ ID NO:90 or a variant thereof; and (ii) a VL having an amino acid sequence of SEQ ID NO: 162 or a variant thereof; or
(i) a VH having an amino acid sequence of SEQ ID NO:91 or a variant thereof; and (ii) a VL having an amino acid sequence of SEQ ID NO: 163 or a variant thereof; or
(i) a VH having an amino acid sequence of SEQ ID NO:92 or a variant thereof; and (ii) a VL having an amino acid sequence of SEQ ID NO:164 or a variant thereof; or
(i) a VH having an amino acid sequence of SEQ ID NO:93 or a variant thereof; and (ii) a VL having an amino acid sequence of SEQ ID NO:165 or a variant thereof.

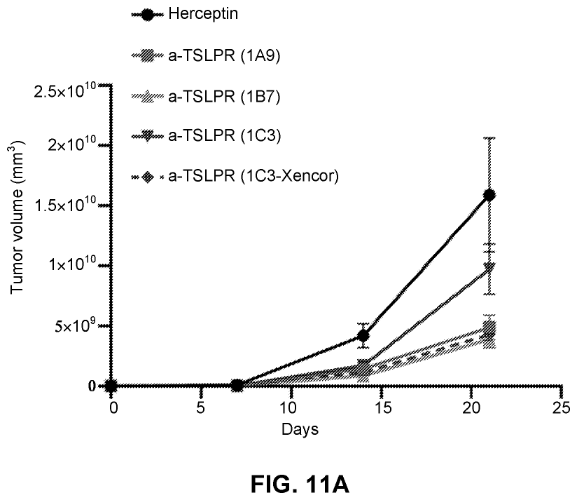

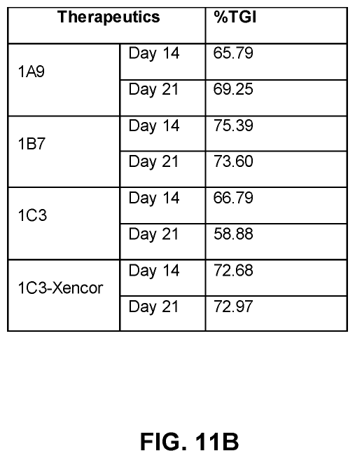

FIG. 19 shows some exemplary Fc domain sequences that are useful with the 2+1 Fab2-scFv-Fc format. The “monomer 1” sequences depicted in FIG. 11 typically refer to the Fc domain of the “Fab-Fc heavy chain” and the “monomer 2” sequences refer to the Fc domain of the “Fab-scFv-Fc heavy chain.” In addition, FIGS. 20 and 21 provide exemplary CH1-hinge domains, CH1 domains, and hinge domains that can be included in the first or second monomer of the 2+1 Fab₂-scFv-Fc format. Further, FIG. 22 provides useful CL sequences that can be used with this format. Exemplary anti-TSLPR x anti-CD3e bispecific antibodies in the 2+1 Fab₂-scFv-Fc format are depicted in FIG. 26.

### VI. Nucleic Acids

In another aspect, provided herein are nucleic acid compositions encoding the anti-TSLPR antibodies, and antigen-binding fragments thereof provided herein. A nucleic acid composition may refer to one or multiple polynucleotides.

As will be appreciated by those in the art, the nucleic acid compositions will depend on the format and scaffold of the heterodimeric protein. Thus, for example, when the format requires three amino acid sequences, such as for the 1+1 Fab-scFv-Fc or 2+1 Fab₂-scFv-Fc formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The antibodies provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VII. Biological and Biochemical Functionality of the Anti-TSLPR x Anti-CD3e Antibodies

Generally, the anti-TSLPR antibodies (e.g., anti-TSLPR x anti-CD3e), and antigen-binding fragments thereof described herein are administered to patients with a TSLPR-associated cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### A. Antibody Compositions for In Vivo Administration

Embodiments of the invention relate to a pharmaceutical composition comprising any one of the anti-TSLPR x anti-CD3e antibodies described herein and a pharmaceutically acceptable carrier. Formulations of the anti-TSLPR x anti-CD3e antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

### VIII. Treatments

Once made, the anti-TSLPR antibodies (e.g., anti-TSLPR x anti-CD3e), and antigen-binding fragments thereof described herein find use in a number of oncology applications. In embodiments, the composition are useful for the treatment of a TSLPR-associated cancer. In some embodiments, the TSLPR-associated cancer is leukemia. In some embodiments, the leukemia is an Acute Lymphoblastic Leukemia, a B-cell Acute Lymphoblastic Leukemia, or a Philadelphia chromosome-like B Cell Acute Lymphoblastic Leukemia. In embodiments, the cancer overexpresses TSLPR. In embodiments, the anti-TSLPR antibodies (e.g., anti-TSLPR x anti-CD3e), and antigen-binding fragments thereof disclosed herein are used to treat allergic reactions. In embodiments, the anti-TSLPR antibodies (e.g., anti-TSLPR x anti-CD3e), and antigen-binding fragments thereof disclosed herein are used to treat asthma. In embodiments, the anti-TSLPR antibodies (e.g., anti-TSLPR x anti-CD3e), and antigen-binding fragments thereof disclosed herein partially block TSLPR-ligand interactions, thereby effectively reducing TSLPR signaling in immune reactions.

### IX. Antibody Compositions for In Vivo Administration

Formulations of the antibodies used in accordance with the antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### X. Administrative Modalities

The antibodies provided herein administered to a subject, in accord with known methods, such as intravenous administration as a bolus or by continuous infusion over a period of time.

### XI. Treatment Modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

### EXAMPLES

### Example 1: TSLPR Expression in Pre-all Samples

Blood samples were obtained from pre-B-ALL patients and cell surface TSLPR and CD19 was labeled with fluorescent antibodies. Expression levels were determined by flow cytometry and confirmed TSLPR and CD19 expression in all ALL patient samples (FIG. 1A-I).

### Example 2: Generation of Monospecific Human Anti-TSLPR Antibodies

Human transgenic mice from Harbour were immunized with human TSLPR protein and single B-cell cloning and expression technology in CHO cells was used to generate antibodies with specific binding activity for TSLPR. Binding affinities for the human TSLPR-binding antibodies were confirmed using SPR/BIAcore. Multiple anti-TSLPR clones were evaluated for their ability to induce activity in antibody-dependent cellular phagocytosis (ADCP) and antibody-dependent cell-mediated cytotoxicity (ADCC) assays.

For standard immunization, purified human TSLPR protein and adjuvant were injected biweekly over a course of a 6-week immunization schedule. Injections were either subcutaneous or intraperitoneal and single B cells were cloned.

For repetitive immunization at multiple sites (RIMMS), animals were repetitively immunized in short intervals at multiple sites, e.g., in proximity to axillary, brachial, superficial inguinal, and popliteal lymph nodes, and lymph nodes at the multiple sites were harvested and used to prepare hybridomas.

Binding affinities for the human TSLPR-binding antibodies were confirmed using SPR/BIAcore. Best binding antibodies were selected and their CDR sequences were cloned into human VH and VL germline variable domains. The CDR sequences and germline variable domains of VH and VL are shown in Tables 9 and 10 below.

TABLE 9
Exemplary VH CDR Sequences and Germline for
TSLPR Antibodies
TSLPR

| | VH | | | | |
|---|---|---|---|---|---|
| | Se- | | | | |
| | quence | VH | | | |
| | SEQ ID | germ- | VH | VH | VH |
| Clone | NO | line | CDR1 | CDR2 | CDR3 |
| 2E4 | 76 | IGHV3- | FTFSSYAMN | GISGSGGSTY | AKDQRITMV |
| | | 23 | SEQ ID | YADSVKG | RGVIG |
| | | | NO: 94 | SEQ ID | SEQ ID |
| | | | | NO: 95 | NO: 96 |
| 2H8 | 77 | IGHV3- | FTFSSHAMN | AISGSGGSTY | ARGHNSGWP |
| | | 23 | SEQ ID | YADSVKG | Y |
| | | | NO: 97 | SEQ ID | SEQ ID |
| | | | | NO: 98 | NO: 99 |
| 3E5 | 78 | IGHV3- | FTFSSNAMS | AIRGRGGSTY | AKDRIVVAG |
| | | 23 | SEQ ID | YADSVKG | PFDH |
| | | | NO: 100 | SEQ ID | SEQ ID |
| | | | | NO: 101 | NO: 102 |
| 1A10 | 79 | IGHV4- | GSISTYYWN | SIYYSGTTNY | ARDQGYYHG |
| | | 59 | SEQ ID | NPSLKS | SGSYFFDY |
| | | | NO: 103 | SEQ ID | SEQ ID |
| | | | | NO: 104 | NO: 105 |
| 1B9 | 80 | IGHV4- | GSIRSSNWWS | EIYHGGTTNN | ARHRYSSGL |
| | | 4 | SEQ ID | NPSLKS | DY |
| | | | NO: 106 | SEQ ID | SEQ ID |
| | | | | NO: 107 | NO: 108 |
| 1D10 | 81 | IGHV4- | GSISRYYWS | YIFYNKNTNY | ARYIAAIYT |
| | | 59 | SEQ ID | NPSLKS | DLYYFDY |
| | | | NO: 109 | SEQ ID | SEQ ID |
| | | | | NO: 110 | NO: 111 |
| 1F6 | 82 | IGHV1- | GTVSSYAIS | GITPIFGTAK | ARGGIAAAG |
| | | 69 | SEQ ID | YAQKFQG | TFDY |
| | | | NO: 112 | SEQ ID | SEQ ID |
| | | | | NO: 113 | NO: 114 |
| 1G3 | 83 | IGHV3- | FTFSSYGMH | VMWYDGRNKY | ARDRIMVRG |
| | | 33 | SEQ ID | YADSVKG | VIIPHVFDI |
| | | | NO: 115 | SEQ ID | SEQ ID |
| | | | | NO: 116 | NO: 117 |
| 3D8 | 84 | IGHV3- | FTFSSYVMH | VIWYAGNKKY | AREYSSSWH |
| | | 30 | SEQ ID | YADSVKG | YYGMDV |
| | | | NO: 118 | SEQ ID | SEQ ID |
| | | | | NO: 119 | NO: 120 |
| 10C4 | 85 | IGHV4- | GSINSNNWWS | DIYHSGSTNY | ARRRWLQHF |
| | | 4 | SEQ ID | NPSLKS | DF |
| | | | NO: 121 | SEQ ID | SEQ ID |
| | | | | NO: 122 | NO: 123 |
| 2A4 | 86 | IGHV1- | YTFTGYYMH | RFNLHSGVTN | ARERQWLVQ |
| | | 2 | SEQ ID | YAQKFQG | DYFDY |
| | | | NO: 124 | SEQ ID | SEQ I |
| | | | | NO: 125 | DNO: 126 |
| 2B2 | 87 | IGHV3- | FTFSNYGMH | FIWYDGSHRY | AKGRGGLDW |
| | | 33 | SEQ ID | YADSVKG | YFDL |
| | | | NO: 127 | SEQ ID | SEQ ID |
| | | | | NO: 128 | NO: 129 |
| 2D11 | 88 | IGHV4- | YSISSGYYWG | RIYHSGTTSY | ARNWGGNWF |
| | | 38 | SEQ ID | NPSLKR | DP |
| | | | NO: 130 | SEQ ID | SEQ ID |
| | | | | NO: 131 | NO: 132 |
| 2E6 | 89 | IGHV3- | FTFGSYVMT | TISASGGSTY | AKDKWGKGF |
| | | 23 | SEQ ID | YADSVKG | DI |
| | | | NO: 133 | SEQ ID | SEQ ID |
| | | | | NO: 134 | NO: 135 |
| 2F1 | 90 | IGHV4- | GSISRYYWS | YIFYNKNTNY | ARYIAAIYT |
| | | 59 | SEQ ID | NPSLKS | DLYYFDY |
| | | | NO: 136 | SEQ ID | SEQ ID |
| | | | | NO: 137 | NO: 138 |
| 2F5 | 91 | IGHV3- | FTFSSHPMN | SISGSGGSTY | AKGHNSGWK |
| | | 23 | SEQ ID | YADFVKG | Y |
| | | | NO: 139 | SEQ ID | SEQ ID |
| | | | | NO: 140 | NO: 141 |
| 2H1 | 92 | IGHV3- | FTFSRYGMH | AISASGGSTY | AKDKWGYAF |
| | | 23 | SEQ ID | YADSVKG | DI |
| | | | NO: 142 | SEQ ID | SEQ ID |
| | | | | NO: 143 | NO: 144 |
| 2H4 | 93 | IGHV1- | GTFNSYAIS | GITPIFGTAK | ARPGIIVAG |
| | | 69 | SEQ ID | YAQKFQG | PFDY |
| | | | NO: 145 | SEQ ID | SEQ ID |
| | | | | NO: 146 | NO: 147 |

TABLE 10
Exemplary VL CDR Sequences and Germline for
TSLPR Antibodies

| | VL | | | | |
|---|---|---|---|---|---|
| | Se- | | | | |
| | quence | VL | | | |
| | SEQ ID | germ- | VL | VL | VL |
| Clone | NO | line | CDR1 | CDR2 | CDR3 |
| 2E4 | 148 | IGKV1- | RASQSISSWLA | KASSLES | QQYVGLIT |
| | | 5 | SEQ ID | SEQ ID | SEQ ID |
| | | | NO: 166 | NO: 167 | NO: 168 |
| 2H8 | 149 | IGKV1- | RASQSISIYLN | AASSLQS | QQSYSTPLT |
| | | 39 | SEQ ID | SEQ ID | SEQ ID |
| | | | NO: 169 | NO: 170 | NO: 171 |
| 3E5 | 150 | IGKV1- | RASQGISSYLA | AASTLQS | QQLNRYPLT |
| | | 9 | SEQ ID | SEQ ID | SEQ ID |
| | | | NO: 172 | NO: 173 | NO: 174 |
| 1A10 | 151 | IGKV3- | RASQSVSSHLA | GASTRAT | QQYNNWPLT |
| | | 15 | SEQ ID | SEQ ID | SEQ ID |
| | | | NO: 175 | NO: 176 | NO: 177 |
| 1B9 | 152 | IGKV1- | RGSHSISNWLG | KASSLKS | QQYNHYST |
| | | 5 | SEQ ID | SEQ ID | SEQ ID |
| | | | NO: 178 | NO: 179 | NO: 180 |
| 1D10 | 153 | IGKV1- | RASQGISSYLA | AASTLQS | QQLNSYPLT |
| | | 9 | SEQ ID | SEQ ID | SEQ ID |
| | | | NO: 181 | NO: 182 | NO: 183 |
| 1F6 | 154 | IGKV1- | RASQSISSWLA | KASSLES | QQYNSYSWT |
| | | 5 | SEQ ID | SEQ ID | SEQ ID |
| | | | NO: 184 | NO: 185 | NO: 186 |
| 1G3 | 155 | IGKV3- | RASQSVSSNLG | GASTRAT | QEYNNWWT |
| | | 15 | SEQ ID | SEQ ID | SEQ ID |
| | | | NO: 187 | NO: 188 | NO: 189 |
| 3D8 | 156 | IGKV1- | RASQGISSYLA | AASTLQS | QQLNSYPLT |
| | | 9 | SEQ ID | SEQ ID | SEQ ID |
| | | | NO: 190 | NO: 191 | NO: 192 |
| 10C4 | 157 | IGKV1- | RASQSITNWLA | KASSLES | QQYNIYPLA |
| | | 5 | SEQ ID | SEQ ID | SEQ ID |
| | | | NO: 193 | NO: 194 | NO: 195 |
| 2A4 | 158 | IGKV1- | RASQSISNYLN | AASSLQS | QQSYSIPPT |
| | | 39 | SEQ ID | SEQ ID | SEQ ID |
| | | | NO: 196 | NO: 197 | NO: 198 |
| 2B2 | 159 | IGKV1- | RASQSISSWLA | KASSLES | LQYNSYSYT |
| | | 5 | SEQ ID | SEQ ID | SEQ ID |
| | | | NO: 199 | NO: 200 | NO: 201 |
| 2D11 | 160 | IGKV1- | RASQGISNYLA | AASTLQS | QKYNSART |
| | | 27 | SEQ ID | SEQ ID | SEQ ID |
| | | | NO: 202 | NO: 203 | NO: 204 |
| 2E6 | 161 | IGKV1- | RASQGISSYLA | AASTLQS | QQLNSYPPT |
| | | 9 | SEQ ID | SEQ ID | SEQ ID |
| | | | NO: 205 | NO: 206 | NO: 207 |
| 2F1 | 162 | IGKV1- | RASQGISSYLA | AASTLqS | QQLNSYPLT |
| | | 9 | SEQ ID | SEQ ID | SEQ ID |
| | | | NO: 208 | NO: 209 | NO: 210 |
| 2F5 | 163 | IGKV1- | RASQGISSYLA | AASTLqS | QQLNSYPPT |
| | | 9 | SEQ ID | SEQ ID | SEQ ID |
| | | | NO: 211 | NO: 212 | NO: 213 |
| 2H1 | 164 | IGKV1- | RASQSISNWLA | KASSLES | LQYNHYST |
| | | 5 | SEQ ID | SEQ ID | SEQ ID |
| | | | NO: 214 | NO: 215 | NO: 216 |
| 2H4 | 165 | IGKV1- | RASQSISSWLA | KASSLES | QQYNSYSWT |
| | | 5 | SEQ ID | SEQ ID | SEQ ID |
| | | | NO: 217 | NO: 218 | NO: 219 |

### Example 3: In Vitro Activity of Monospecific TSLPR Antibodies

### Affinity Assays

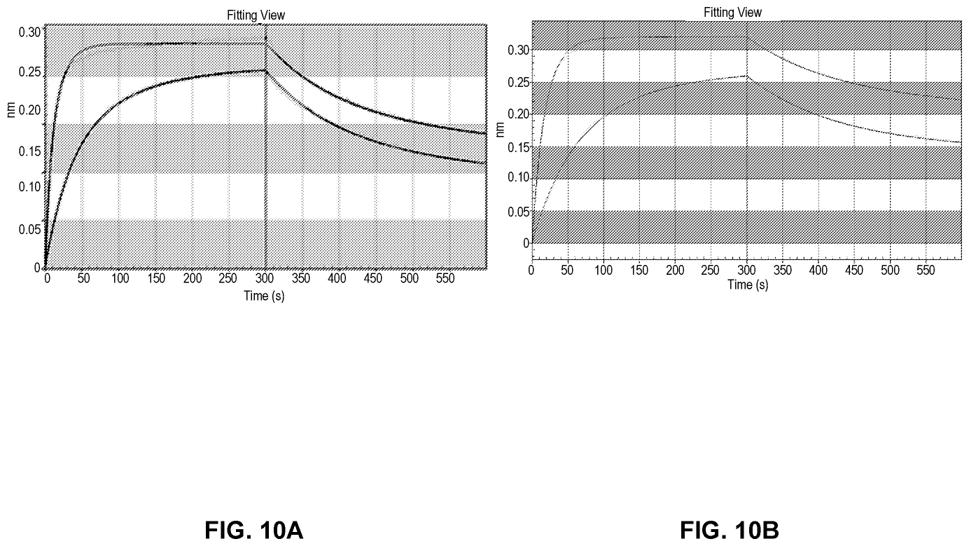

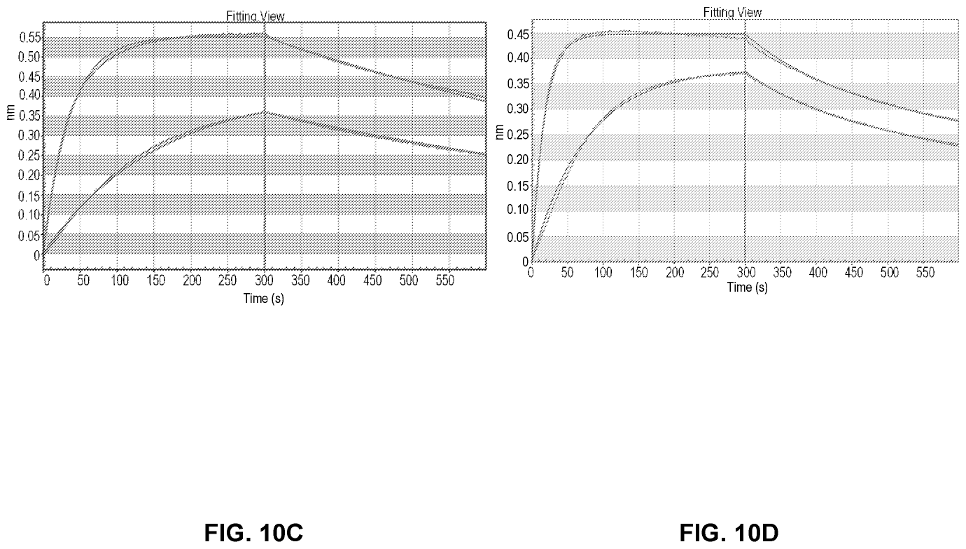

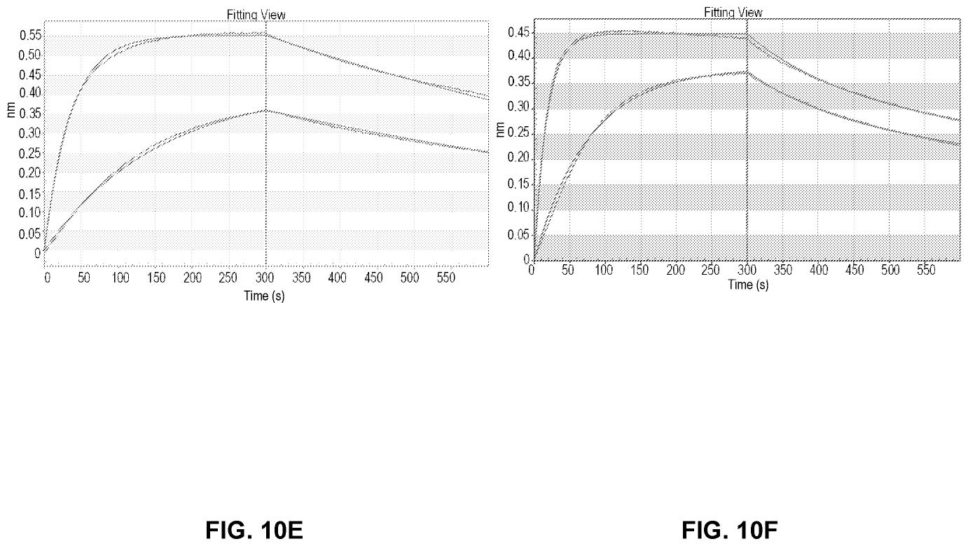

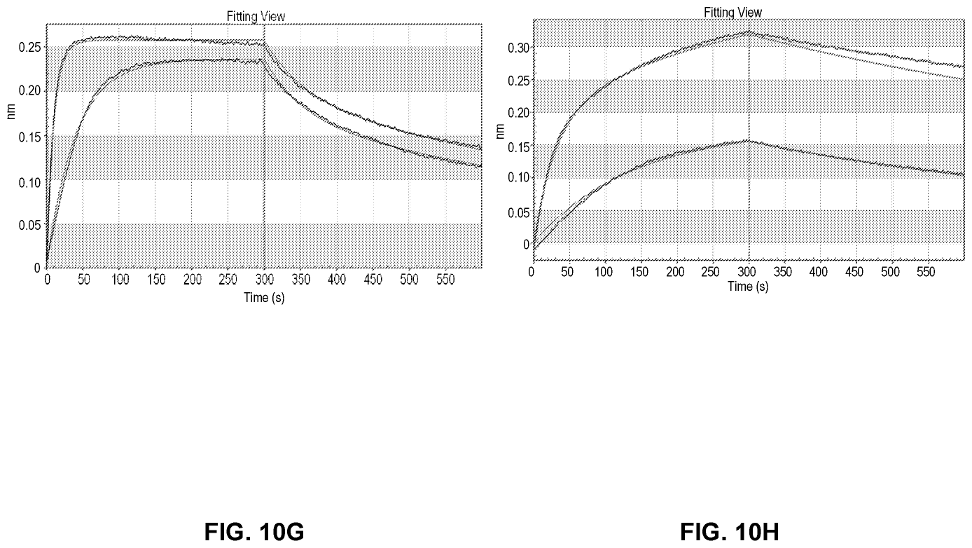

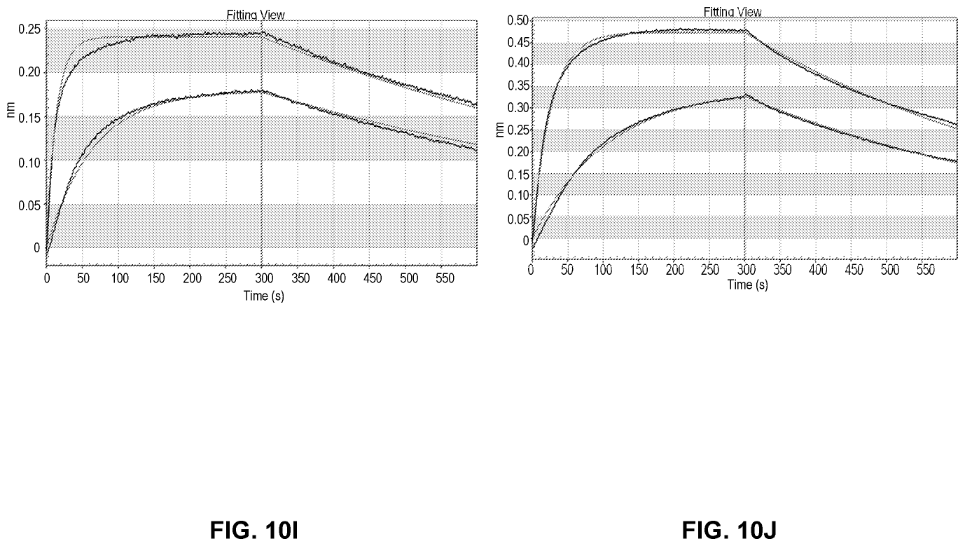

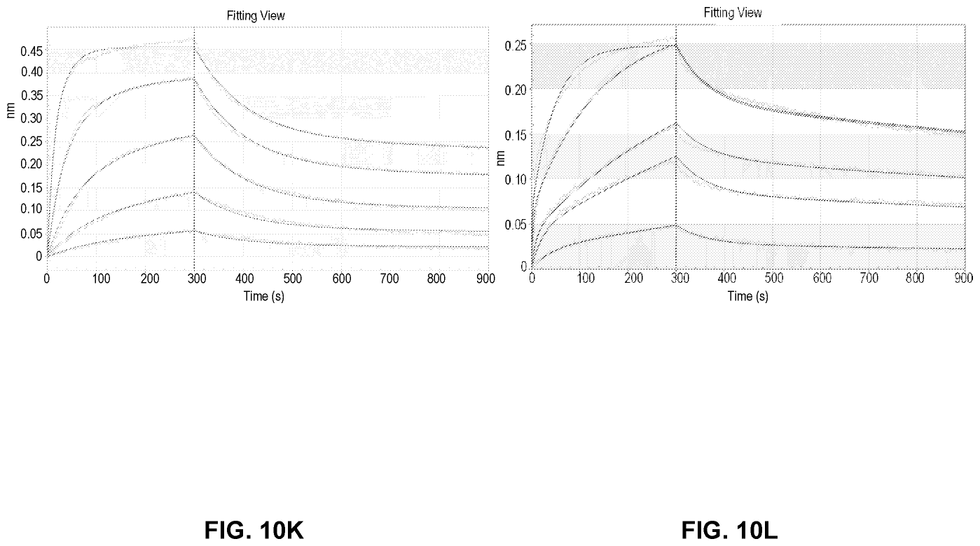

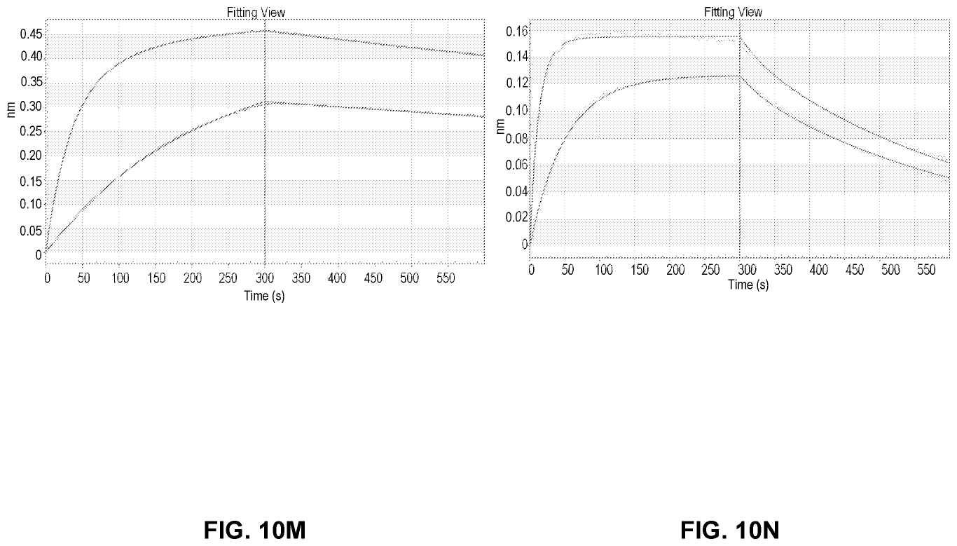

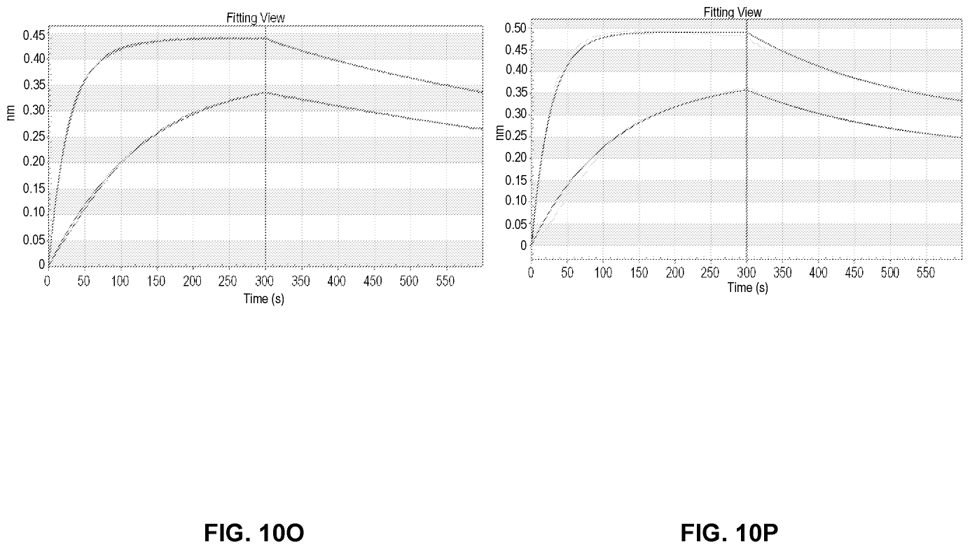

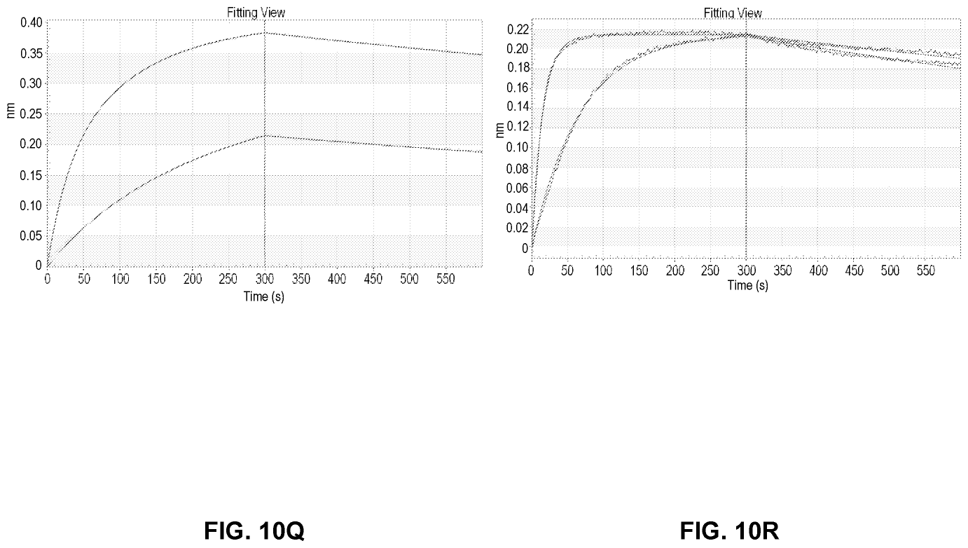

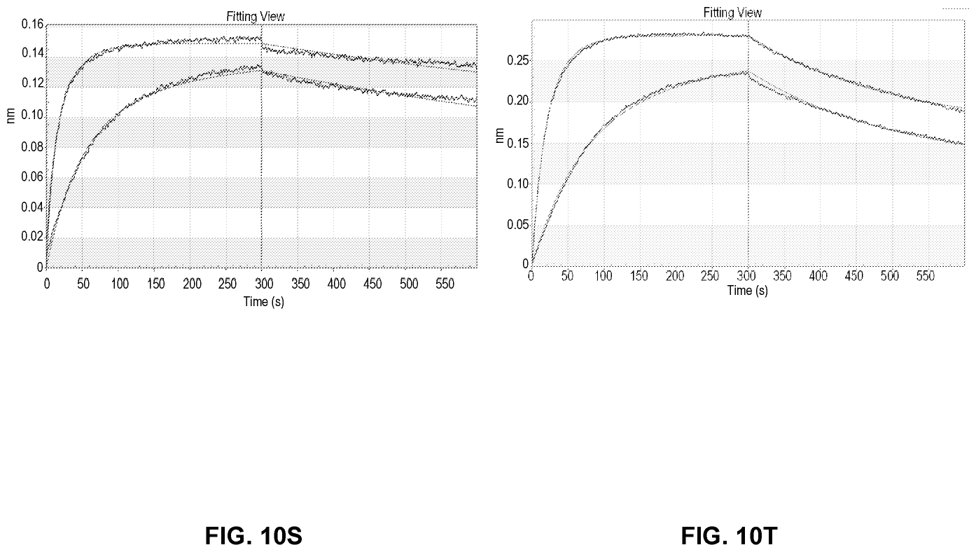

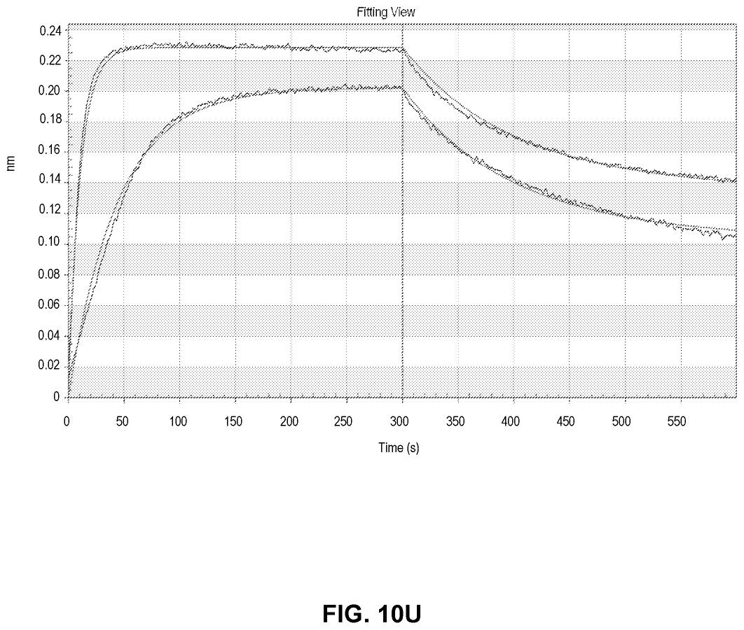

A ForteBio prefunctionalized AHQ (anti-human IgG quantitation sensor) was used to determine affinities of the anti-TSLPR antibodies disclosed in Tables 9 and 10. TSLPR antibody clones whose VH and VL sequences are disclosed in Tables 9 and 10 were bound to the AHQ sensor and his-tagged TSLPR (TSLPR-HIS) was provided in solution. For competition studies, TSLP was also added in solution. The anti-TSLPR antibodies were serially diluted with an HBS-EP solution (GE Healthcare), and 100 μL of the solution was added to the flow path at a flow rate of 50 μUmin. Binding rate constant (kon), dissociation rate constant (koff), and dissociation constant (KD) of the anti-TSLPR antibody clones were calculated using data analysis software (BIA Evaluation). ForteBio binding profiles for TSLPR clones are shown in FIGS. 10A-10U.

Table 11 provides the dissociation constants (K_{D}) and rate constants of association (kon) and dissociation (koff) for each of the TSLPR clones with VH and VL sequences provided in Tables 9 and 10.

In competition studies, TSLPR antibody bound sensors were incubated with TSLPR-HIS and TSLP in solution and blockers and partial blockers were identified.

All TSLPR clones tested demonstrated high affinity binding to TSLPR as indicated by low K_{D} values (Table 11). Furthermore, several TSLPR clones demonstrated tighter binding to TSLPR (i.e., lower K_{D} values) in the presence of TSLP indicating blocking activity towards the ligand-occupied TSLP receptor (see, e.g., clones 1A9.2, 1G3, 2A4, 2B2, 2H4 and 1C3 in Table 11).

TABLE 11
Functional Characterization of TSLPR Antibodies
TSLPR-

| | | | | Response of | TSLP |
|---|---|---|---|---|---|
| | IgG K_{D} | kon | koff | TSLPR-HIS | Response |
| Clone | (M) | (1/Ms) | (1/s) | to mAbs (nM) | (nM) |
| 2E4 | 1.01E−09 | 2.92E+05 | 2.94E−04 | 0.2399 | 0.3732 |
| 2H8 | 8.06E−10 | 2.37E+05 | 1.91E−04 | 0.3255 | 0.5008 |
| 3E5 | 6.81E−09 | 2.04E+05 | 1.39E−03 | 0.3690 | 0.5467 |
| 1A9 | 4.45E−09 | 1.73E+05 | 7.69E−04 | 0.5808 | 0.2142 |
| | | | | | Blocker |
| 1A10 | 8.90E−09 | 1.33E+05 | 1.19E−03 | 0.5579 | 0.6189 |
| 1B7 | 2.82E−09 | 2.80E+05 | 7.90E−04 | 0.4395 | 0.3331 |
| 1B9 | 2.33E−09 | 5.45E+05 | 1.27E−03 | 0.2534 | 0.3536 |
| 1D10 | <1.0E−12 | 1.62E+04 | <1.0E−07 | 0.3215 | 0.5440 |
| 1F6 | 3.90E−09 | 3.51E+05 | 1.37E−03 | 0.2499 | 0.3541 |
| 1G3 | 1.17E−08 | 1.79E+05 | 2.10E−03 | 0.4782 | 0.2135 |
| | | | | | Blocker |
| 3D8 | 1.16E−09 | 1.16E+05 | 1.34E−04 | | |
| 10C4 | 4.86E−09 | 6.88E+04 | 3.34E−04 | | |
| 2A4 | 3.07E−09 | 1.06E+05 | 3.26E−04 | | −3.7% |
| | | | | | Blocker |
| 2B2 | 8.07E−09 | 4.01E+05 | 3.24E−03 | | 21.2% |
| | | | | | Blocker |
| 2D11 | 5.29E−09 | 1.67E+05 | 8.81E−04 | | 83.2% |
| 2E6 | 7.63E−09 | 1.84E+05 | 1.40E−03 | | 82.6% |
| 2F1 | 4.33E−09 | 7.68E+04 | 3.32E−04 | | 204% |
| 2F5 | 1.28E−09 | 3.51E+05 | 4.50E−04 | | 120% |
| 2H1 | 1.29E−09 | 3.55E+06 | 4.59E−04 | | 80% |
| 2H4 | 5.91E−09 | 2.54E+05 | 1.50E−03 | | 32.2% |
| | | | | | Partial |
| | | | | | Blocker |
| 1C3 | 1.10E−09 | 2.44E+05 | 2.69E−04 | | 44% |
| | | | | | Blocker |

### ADCC Assays

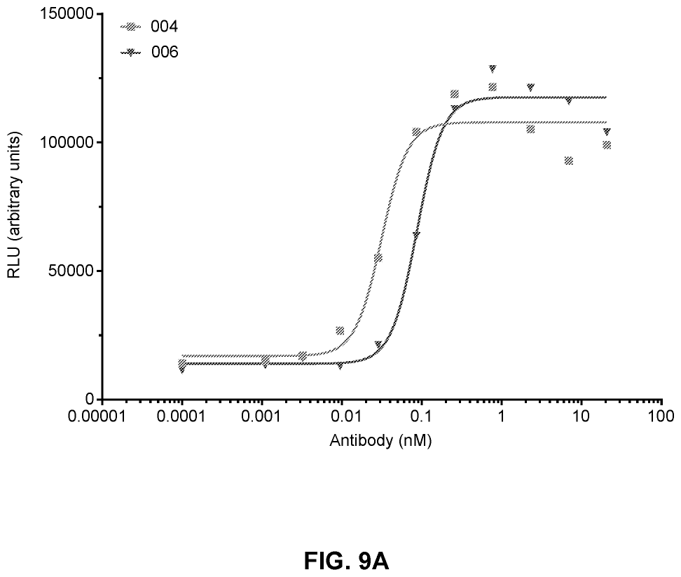

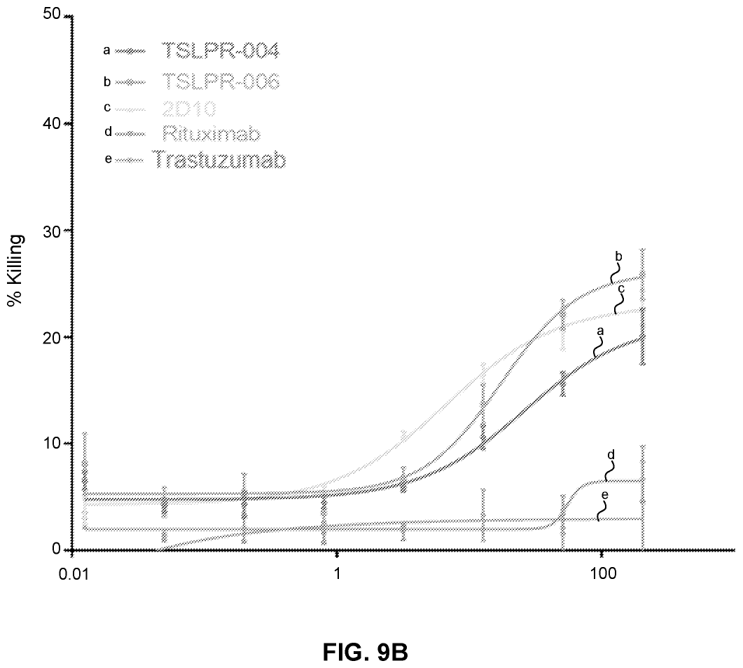

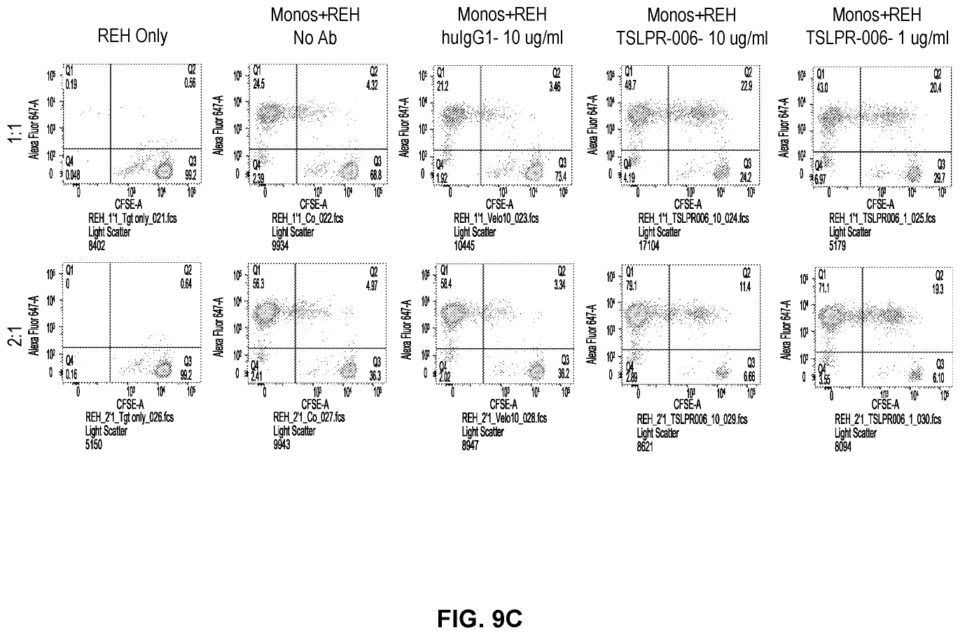

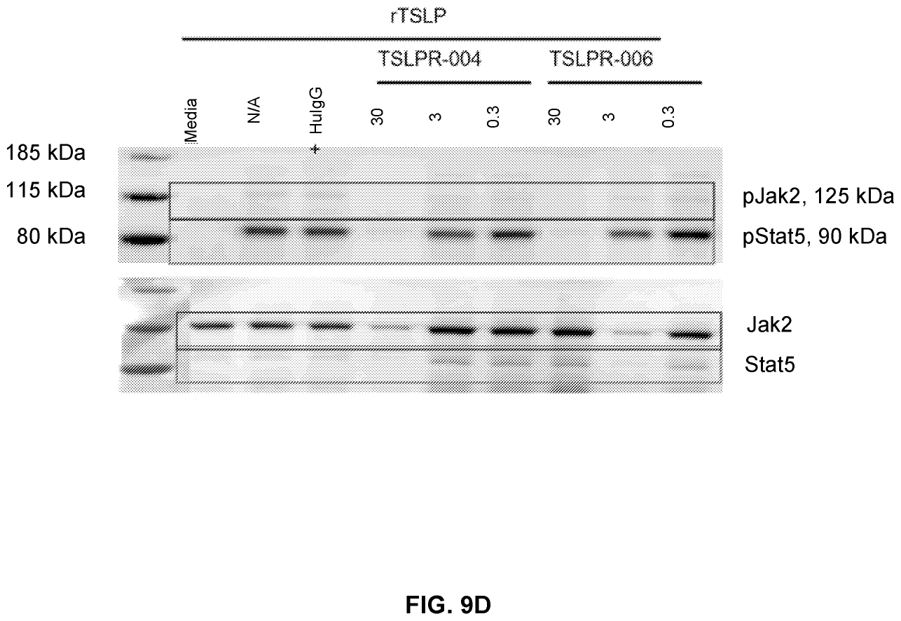

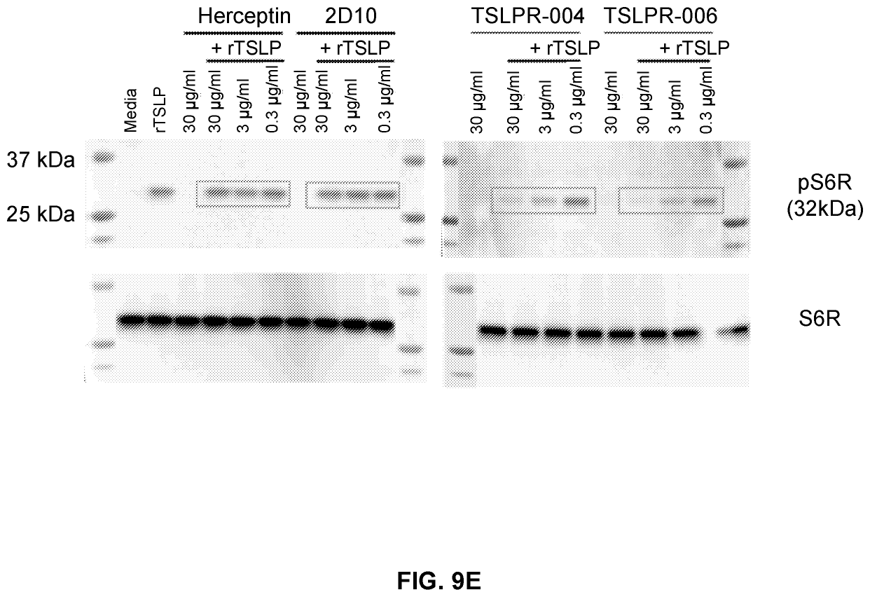

Multiple anti-TSLPR clones were screened for their ability to induce activity in a Promega ADCC assay. The clones were added at the indicated concentrations to TSLPR-expressing REH cells (receptor #as high as 32,000 sites per cell) before adding reporter cells at an effector:target (E:T) ratio of 10:1 and cultured overnight. The assay was performed as recommended by the manufacturer (Promega, Madison, WI). Two antibodies, TSLPR-004 and TSLPR-006, induced strong signals at an EC₅₀ of 0.03 and 0.08 nM, respectively, indicating strong ADCC activity (FIG. 9A).

Further, a primary ADCC assay was performed using a Calcein labeling method. TSLPR-expressing REH cells were labeled with Calcein (10 μM; Sigma Aldrich, St. Louis, MO) for 30 mins at 37° C. and used as targets. The cells were washed and allowed to rest in complete RPMI medium for two hours before washing them twice and counting. Using 96 well plates, 5000 labeled REH-TSLPR cells were added per well followed by addition of TSLPR-004, TSLPR-006, 2D10, Rituximab, and Trastazumab antibodies at varying amounts and incubating for 1 hr at 4° C. Herceptin and Rituximab were used as negative controls while 2D10 clone (anti-TSLPR antibody) was used as a positive control. PBMCs were added at a effector:target ratio (E:T) of 30:1 and the plates were incubated at 37° C. for 4 hrs. Fluorescence signals in the supernatants were measured to determine cell lysis. Data showed modest killing of REH TSLPR⁺ cells by all three anti-TSLPR clones tested that induced around 20% cell death in 4 hrs at the highest dose. On the other hand, no cell killing was observed with Rituximab or Trastuzumab antibodies, suggesting specific killing of TSLPR-expressing REH cells by anti-TSLPR antibodies (FIG. 9B).

### Primary ADCP Assays

CD14⁺ and CD16⁺ monocytes were isolated from human PBMCs by negative selection using the EasySep™ Human Monocyte Enrichment Kit without CD16 Depletion (Stem Cell Technology, Vancouver, Canada), plated in X-VIVO™ 15 media (Lonza, Basel Switzerland) supplemented with 10% FBS and 25 ng/ml recombinant human M-CSF (R&D Systems, Minneapolis, MN), and allowed to differentiate for seven days in a standard tissue culture flask. The media and M-CSF were refreshed at day 4. Cells were detached non-enzymatically for use as effectors in an in vitro ADCP assay using TSLPR-expressing REH cells as targets. Target cells were labeled with 2 μM CFSE (Life Technologies, Carlsbad, CA), washed, suspended in warm RPMI containing 10% FBS, then incubated with an isotype control at 10 μg/ml or TSLPR-006 antibodies at 1 and 10 μg/ml for 20 minutes at room temperature. Differentiated monocytes were added at effector:target ratios of 1:1 or 2:1, with targets held constant at 0.2 million per well in a 48-well plate. The ADCP assay was incubated for two hours at 37° C. Afterwards, cell suspensions were harvested and washed in FACs buffer containing 2% FBS, normal goat serum, and normal rabbit serum (all from Millipore Sigma, St. Louis, MO). Subsequently, samples were FcR blocked using Fc Receptor Binding Inhibitor (Invitrogen/Thermo Fisher) for 15 minutes at 4° C. and stained with anti-huCD11b Alexa 647 for 30 minutes at 4° C. Samples were washed and prepared for readout on an LSR II Fortessa (BD Biosciences, San Jose, CA). Cells were analyzed to determine tumor cells alone (CFSE⁺, CD11b⁺), macrophages alone (CFSE⁺, CD11b⁺), and phagocytosed tumor cells (CFSE⁺, CD11b⁺). Percent phagocytosis was determined using the following equation: (phagocytosed tumor cells)/(phagocytosed tumor cells+tumor cells alone)×100%. Results showed that TSLPR-006 antibodies significantly increased target phagocytosis in all conditions tested (Monos+REH TSLPR-006 10 μg/ml and 1 μg/ml) while REH cells alone (REH only) and REH cell/monocyte mixtures with only media (Monos+REH No AB) or an unrelated human IgG1 antibody (Monos+REH huIgG1-10 μg/ml) showed only basal levels of phagocytosis (FIG. 9 C).

Previous analyses using Octet® Detection Systems for Biomolecular Interaction Analysis had suggested that clones TSLPR-004 and TSLPR-006 were capable of blocking TSLPR interaction with its ligand, at least partially. To confirm this finding at the cellular level, clone TSLPR-004 and TSLPR-006 antibodies were used to block TSLP-mediated signaling through TSLPR. TSLPR-expressing REH cell lines were coated with anti-TSLPR antibodies 004 and 006 before stimulating the cells with recombinant TSLP. Specifically, one million TSLPR-expressing REH cells were plated per well of a 48-well plate in 1 ml of serum-free RPMI medium and cultured overnight. The next day, anti-TSLPR or isotype control antibodies were added at varying concentrations for 30 minutes on ice. Afterwards, recombinant TSLP (R&D Systems, Minneapolis, MN) was added at 25 ng/ml to the relevant wells and incubated at 37° C. for 60 minutes. Subsequently, cell suspensions were collected, pelleted, and lysed in 50 μL of phosphatase-inhibitor containing lysis buffer. The lysates were quantitated using a BCA assay (Pierce/Thermo Fisher) and normalized to 1 mg/ml with 4× sample buffer containing 2-mercaptoethanol. Lysates were run in reduced format on 4-12% Bis-Tris gels (Invitrogen) at 20 μg of total protein per lane, transferred using the iBlot2 dry transfer system (Invitrogen), then blocked using 5% non-fat dry milk in Tris-buffered saline containing 0.05% Tween-20. Cell lysates were tested by Western blot using TSLPR downstream signaling components: pJak2, pStat5 and pS6R. Primary antibody incubations with anti-phospho Jak2 (Tyr1007), Stat5 (Tyr694), or S6R (Ser235/236) or total forms of the same (Cell Signaling Technology, Danvers, MA) were performed overnight at 4° C. with gentle rocking, using the manufacturer's recommended dilutions. Washes were performed with Tris-buffered saline containing 0.05% Tween-20 and anti-species appropriate HRP-conjugated secondary antibodies (Cell Signaling Technology) were added for a 60-minute incubation with gentle rocking at room temperature. Subsequent to further washes, the blots were developed using ECL Prime Western Blotting Detection Reagent (Cytiva Amersham, Marlborough, MA) for approximately 60 seconds and imaged using the c600 Imaging System (Azure Biosystems, Dublin, CA). Data showed that TSLP treatment induced phosphorylation of Jak2, Stat5 (FIG. 9 D) and pS6R (FIG. 9 E) and that the phosphorylation was inhibited by TSLPR-004 and TSLPR-006 antibodies in a dose dependent manner but not by control antibody 2D10. These data confirmed that TSLPR-004 and TSLPR-006 antibodies were capable of partially blocking the interaction of TSLP with its receptor and inhibiting TSLPR downstream signaling.

### Example 4: Generation of Bispecific TSLPR X CD3 Antibodies

Bispecific TSLPR x CD3 antibodies that contain two binding moieties, one that binds the cancer/leukemia-associated antigen TSLPR on B-ALL cells and the other that binds CD3 on T cells were generated. An IgG format was chosen for long half-life in vivo, its ready production in CMC, and its overall safety profile for use as therapeutic agent in humans. Specifically, the bispecific TSLPR x CD3 antibodies were generated in an Fab format for the TSLPR binding site and a scFv-Fc format for the CD3 binding site and were constructed as follows.

Human transgenic mice from Harbour were immunized with human TSLPR protein and single B-cell cloning and expression technology in CHO cells was used to generate antibodies with specific binding activity for TSLPR. Binding affinities for the human TSLPR-binding antibodies were determined using BIAcore and antibodies with TSLPR binding affinities with a Kd of about 2.872 nM for human TSLPR and a Kd of about 2.03 nM for cynomolgus TSLPR and partial TSLP binding blocking activity were selected for further development of bispecific antibodies (1B7 antibody). The VL and VH sequences of the anti-TSLPR moieties of the anti-human TSLPR antibodies were synthesized as gBlocks (Integrated DNA Technologies).

VL and VH sequences of an anti-CD3 epsilon domain-binding moiety of an anti-human CD3 antibody were also synthesized as gBlocks (Integrated DNA Technologies) and a (Gly₄Ser)₃ linker encoding sequence was used to fuse the VL and VH of the CD3 binding moiety by overlap extension PCR.

The TSLPR binding and CD3 binding moieties were engineered into TSLPR x CD3 bispecific antibodies using heterodimer Fc technology. The segment encoding the Fc domain, derived from human IgG1, was further modified to contain an N297A mutation to prevent glycosylation in the CH2 domain, and either glutamic acid, a serine, or an aspartic acid mutation in one CH3 domain and a glutamine, a lysine, a leucine, or an asparagine mutation in the opposing CH3 domain were introduced to allow for optimal heterodimerization.

The purity of the prepared TSLPR x CD3 antibodies was determined using Ultra Performance Liquid Chromatography (UPLC™) and a purity of 95% was detected (FIG. 2C). Mass spectrometry identified the whole bispecific TSLPR x CD3 molecule (FIG. 2D).

The sequences of the heavy and light chain variable regions of the bispecific TSLPR x CD3 antibodies generated are listed below, with CDR sequences identified above in Tables 3-8.

1B7 bispecific TSLPR binding site heavy
chain sequence:
(SEQ ID NO: 31)
QVQLQESGPGLVKPSGTLSLTCAVSGGSISSSNWWSWVRQPPGKGLE
WIGEIYHGGTTNNNPSLKSRVTISVDKSKSQFSLKLSSVTAADTAVYYCA
RHRYSNGLDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSDTKVDKKV//EPKSCDKTHTCPPCPAPPVAGPSVFLFPP
KPKDTLMISRTPEVTCVVVDVKHEDPEVKFNWYVDGVEVHNAKTKPREE/
EYNSTYRVVSVLTVLHQDWLNGKEYKCKVSNKALPAPIEKTISKAKGQPR
EPQVYTLPPSREEMTKNQVSLTCDVSGFYPSDIAVEWESDGQPENNYKTT
PPVLDSDGSFFLYSKLTVDKSRWEQGDVFSCSVMHEALHNHYTQKSLSLS
PG
1B7 bispecific TSLPR binding site light
chain sequence:
(SEQ ID NO: 32)
DIQMTQSPSTLSASVGDRVTITCRASQSISNWLAWYQQKPGKAPKLLI
YKASSLESGVPSRFSGSGSGTEFTLTISSLQPDDFATYYCQQYNHYSTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.
Bispecific CD3 binding site scFv sequence:
(SEQ ID NO: 33)
EVQLVESGGGLVQPGGSLRLSCAASGFTFSTYAMNWVRQAPGKGLE
WVGRIRSKYNNYATYYADSVKGRFTISRDDSKNTLYLQMNSLRAEDTAVY
YCVRHGNFGDSYVSWFAYWGQGTLVTVSS/GKPGSGKPGSGKPGSGKPGS
QAVVTQEPSLTVSPGGTVTLTCGSSTGAVTTSNYANWVQQKPGKSPRGLI
GGTNKRAPGVPARFSGSLLGGKAALTISGAQPEDEADYYCALWYSNHWVF
GGGTKLTVL//EPKSSDKTHTCPPCPAPPVAGPSVFLFPPKPKDTLMISR
TPEVTCVVVDVKHEDPEVKFNWYVDGVEVHNAKTKPREEQYNSTYRVVSV
LTVLHQDWLNGKEYKCKVSNKALPAPIEKTISKAKGQPREPQVYTLPPSR
EQMTKNQVKLTCLVKGFYPSDIAVEWESNGQPENNYKTTPPVLDSDGSFF
LYSKLTVDKSRWQQGNVFSCSVMHEALHNHYTQKSLSLSPGK

Nucleic acid sequences for heavy chain variable domains are listed below.

TSLPR-4 VH DNA (1A9)
(SEQ ID NO: 34)
ATGAAACACCTGTGGTTCTTCCTCCTGCTGGTGGCAGCTCCCAGAT
GGGTCCTGCCCCAGGTGCAGCTGCAGGAGTCGGGCCCAGGACTGGTGAAG
CCTTCGGAGACCCTGTCCCTCACCTGCGCTGTCTCTGGTTACTCCATCAG
CAGTGGTTACTACTGGGGCTGGATCCGGCAGCCCCCAGGGAAGGGGCTGG
AGTGGATTGGGAGGATCTATCATAGTGGGACCACCTCCTACAACCCGTCC
CTCAAGAGACGAGTCACCATTTCAGTTGACACGTCCAAGAATCAGTTCTC
CCTGAAACTGAGCTCTGTGACCGCGACAGACACGGCCGTGTATTACTGTG
CGAGAAACTGGGGAGGGAACTGGTTCGACCCCTGGGGCCAGGGAACCCTG
GTCACCGTCTCGAGT
TSLPR-6 VH DNA (1B7)
(SEQ ID NO: 35)
ATGAAACACCTGTGGTTCTTCCTCCTCCTGGTGGCAGCTCCCAGAT
GGGTCCTGTCCCAGGTGCAGCTGCAGGAGTCGGGCCCAGGACTGGTGAAG
CCTTCGGGGACCCTGTCCCTCACCTGCGCTGTCTCTGGTGGCTCCATCAG
CAGTAGTAACTGGTGGAGTTGGGTCCGCCAGCCCCCAGGGAAGGGGCTGG
AGTGGATTGGGGAAATCTATCATGGTGGGACCACCAACAACAACCCGTCC
CTCAAGAGTCGAGTCACCATATCAGTAGACAAGTCCAAGAGCCAGTTCTC
CCTGAAACTGAGCTCTGTGACCGCCGCGGACACGGCCGTGTATTACTGTG
CGAGACACCGGTATAGTAATGGCCTTGACTACTGGGGCCAGGGAACCCTG
GTCACCGTCTCGAGT
TSLPR-21 VH DNA (1C3)
(SEQ ID NO: 36)
ATGGACTGGACCTGGAGGATCCTCTTCTTGGTGACAGCAGCCACAG
GAGCCCACTCCCAGGTGCAACTGGTGCAGTCTGGGGCTGAGGTGAAGAAG
CCTGGGGCCTCGGTGAAGGTCTCCTGCAAGGCTTCTGGATACACCTTCAG
CGGCTATTATATGCATTGGGTGCGACAGGCCCCTGGACAAGGGCTTGAGT
GGATGGGAAGGTTCAACCTTCACGGAGGTGTCGCAGACTATGCACAGAAG
TTTCAGGGCAGGGTCACCATGACCAGGGACACGTCCATCAGCACAGCCTA
CATGGAGCTGAGCAGGCTGCGATCTGACGACACGGCCGTGTATTACTGTG
TGAGAGAACGGCAGTGGCTGGTACAGGACTACTTTGACTACTGGGGCCAG
GGAACCCTGGTCACCGTCTCGAGT

Nucleic acid sequences for light chain variable domains are listed below.

TSLPR-4 VL DNA (1A9)
(SEQ ID NO: 37)
ATGGAAGCCCCAGCGCAGCTTCTCTTCCTCCTGCTACTCTGGCTCCC
AGATACCACCGGAGAAATTGTGTTGACACAGTCTCCAGCCACCCTGTCTT
TGTCTCCAGGGGAAAGAGCCACCCTCTCCTGCAGGGCCAGTCAGAGTGTT
AGCAGCTACTTAGCCTGGTACCAACAGAAACCTGGCCAGGCTCCCAGGCT
CCTCATCTATGATGCATCCAACAGGGCCACTGGCATCCCAGCCAGGTTCA
GTGGCAGTGGGTCTGGGACAGACTTCACTCTCACCATCAGCAGCCTAGAG
CCTGAAGATTTTGCAGTTTATTACTGTCAGCAGCGTAGCACCTGGCTCAC
TTTCGGCGGAGGGACCAAAGTGGATATCAAACGTACGGTGGCCGCTCCA
TSLPR-6 VL DNA (1B7)
(SEQ ID NO: 38)
ATGAGGCTGCCAGCACAGCTGCTGGGACTGCTGATGCTGTGGGTGA
GCGGCAGCTCCGGCGACATCCAGATGACCCAGTCCCCTTCTACACTGAGC
GCCTCCGTGGGCGATAGGGTGACCATCACATGCAGAGCCTCTCAGAGCAT
CTCCAACTGGCTGGCCTGGTACCAGCAGAAGCCAGGCAAGGCCCCCAAGC
TGCTGATCTATAAGGCCTCTAGCCTGGAGTCTGGCGTGCCTAGCAGGTTC
TCTGGAAGCGGATCCGGAACCGAGTTTACCCTGACAATCTCCTCTCTGCA
GCCAGACGATTTCGCCACATACTATTGCCAGCAGTACAATCACTATTCCA
CCTTTGGCCAGGGCACAAAGCTGGAGATCAAGCGTACGGTGGCCGCCCCA
TSLPR-21 VL DNA (1C3)
(SEQ ID NO: 39)
ATGGACATGAGGGTCCCCGCTCAGCTCCTGGGGCTCCTGCTACTCT
GGCTCCGAGGTGCCAGATGTGACATCCAGATGACCCAGTCTCCATCTTCC
CTGTCTGCATCTGTAGGAGACAGAGTCACCATCACTTGCCGGGCAAGTCA
GAGCATTAGCAGGTCTTTAAATTGGTATCAGCAGAAACCAGGGAAAGCCC
CTCAGCTCCTGATCTATGCTGCCTCCAGTTTGCAAAGTGGGGTCCCATCA
AGGTTCAGTGGCAGTGGATCTGGGACAGATTTCACTCTCACCATCAGCAG
TCTGGAACCTGAAGATTTTGCGACTTACCACTGTCAACAGAGTTACAGTA
TTTCGGTCACTTTCGGCGGAGGGACCAAAGTGGATATCAAACGTACGGTG
GCCGCTCCA

Amino acid sequences for heavy chain variable domains are listed below.

TSLPR-4 VH Protein (1A9)
(SEQ ID NO: 9)
QVQLQESGPGLVKPSETLSLTCAVSGYSISSGYYWGWIRQPPGKGLEW
IGRIYHSGTTSYNPSLKRRVTISVDTSKNQFSLKLSSVTATDTAVYYCAR
NWGGNWFDPWGQGTLVTVSS
TSLPR-6 VH Protein (1B7)
(SEQ ID NO: 7)
QVQLQESGPGLVKPSGTLSLTCAVSGGSISSSNWWSWVRQPPGKGLE
WIGEIYHGGTTNNNPSLKSRVTISVDKSKSQFSLKLSSVTAADTAVYYCA
RHRYSNGLDYWGQGTLVTVSS.
TSLPR-21 VH Protein (1C3)
(SEQ ID NO: 8)
QVQLVQSGAEVKKPGASVKVSCKASGYTFSGYYMHWVRQAPGQGLE
WMGRFNLHGGVADYAQKFQGRVTMTRTSISTAYMELSRLRSDDTAVYYCV
RERQWLVQDYFDYWGQGTLVTVSS

Amino acid sequences for light chain variable domains are listed below.

TSLPR-4 VL Protein (1A9)
(SEQ ID NO: 12)
EIVLTQSPATLSLSPGERATLSCRASQSVSSYLAWYQQKPGQAPRLLIY
DASNRATGIPARFSGSGSGTDFTLTISSLEPEDFAVYYCQQRSTWLTFGG
GTKVDIKRTVAAP.
TSLPR-6 VL Protein (1B7)
(SEQ ID NO: 10)
DIQMTQSPSTLSASVGDRVTITCRASQSISNWLAWYQQKPGKAPKLLI
YKASSLESGVPSRFSGSGSGTEFTLTISSLQPDDFATYYCQQYNHYSTFG
QGTKLEIKRTVAAP
TSLPR-21 VL Protein (1C3)
(SEQ ID NO: 11)
DIQMTQSPSSLSASVGDRVTITCRASQSISRSLNWYQQKPGKAPQLLIY
AASSLQSGVPSRFSGSGSGTDFTLTISSLEPEDFATYHCQQSYSISVTFG
GGTKVDIKRTVAAP

### Example 5: Characterization of Binding Activity of the Bispecific TSLPR X CD3 Antibodies

SPR analysis of bispecific TSLPR x CD3 antibodies was performed using BIAcore™ 2000 (GE Healthcare). Respective human TSLPR proteins or human CD3-Fc fusion proteins were immobilized on the surface of a sensor chip CMS using a Capture Kit and an Amine Coupling Kit (GE Healthcare). The bispecific TSLPR x CD3 antibodies obtained in Example 2 were serially diluted with an HBS-EP solution (GE Healthcare), and 100 μL of the solution was added to the flow path at a flow rate of 50 μUmin. By this measurement system, binding rate constant (kon), dissociation rate constant (k_{dis}), and dissociation constant (KD) of the TSLPR x CD3 antibodies and human TSLPR or human CD3 were calculated using data analysis software (BIA Evaluation) (FIGS. 2A and 2B). The BIAcore™ studies documented that the TSLPR x CD3 antibodies had binding functions for both, human TSLPR (FIG. 2A) and human CD3 (FIG. 2B).

### Example 6: Cytotoxicity Assays

The bispecific TSLPR x CD3 antibodies with the best cytotoxicity were selected for further characterization. Efficacy of TSLPR x CD3 bispecific antibodies to induce ALL Blast Lysis in Primary ALL Patient Samples was tested. ALL blasts were incubated with PBMC and different concentrations of monospecific TSLPR antibodies and bispecific TSLPR x CD3 antibodies. Efficient cell lysis was only observed in the presence of TSLPR x CD3 bispecific antibodies but not in the presence of TSLPR monospecific antibodies (FIG. 2E).

### Example 7: In Vivo Efficacy of TSLPR Bispecific Antibodies in Patient-Derived Xenograft (PDX)

### Animal, Dosing, and Tumor/Leukemia Cell Accumulation Measurement

The study protocol was approved by MDACC after review by the Animal Care and Use Committee of the AAALAC-certified institution. Single dose PK of anti-TSLPR x CD3 was studied in adult, female NSG (the Jackson Laboratory) mice as well as PBMC humanized NSG mice. For the PK in NSG mice, animals were randomized into treatment groups, and were ip injected with the bispecific antibody at 0.1, 0.3 and 1 mg/kg (n=5). For PK in humanized NSG mice, 1×10⁷ PBMC were iv injected into the mice 10 days prior to randomization for dosing. Blood was collected at indicated time points post injection through serial sampling and processed to plasma for bioanalytical and PK analyses. Concentrations of anti-TSLPR x CD3 in plasma samples were measured using antigen coated ECL based ELISA (MSD, MD) as described. PK analyses were performed according to standard non-compartmental analysis using WinNonlin package (v. 6.3, Pharsight, Certara Company).

NOD.Cg-Prkdcscid Il2rgtm 1Wjl/SzJ (NSG) mice (the Jackson Laboratory) were intravenously (IV) injected with 5000 U937-A2-Luc (Luciferase), 5M EM2-Luc and 1×10⁵ ML2-Luc human AML/CML cells. Luciferase-labeled patient-derived ALL blasts and PBMC were from two different donors (PBMC-296, FIG. 3 left panels, and PBMC-879, FIG. 3, right panels). Tumor/leukemia engraftment was assessed by imaging using a Xenogen Imaging System (PerkinElmer). Once the tumor/leukemia blast accumulations established, 10M PBMC per mouse were IV injected for humanization. Three to ten days after PBMC injection, mice were randomized into control and treatment groups and then either treated with control or anti-TSLPR x CD3 antibody at different doses via intraperitoneal (IP) injection weekly for three weeks.

### T Cell Dynamic, Activation, and Phenotyping

Mice were bled weekly through tail vein to monitor the CD3⁺T cell dynamic changes. Briefly, mouse blood was processed to single cell suspension and stain with Ghost dye (BV510, Tonbo Biosciences), mCD45 (Invitrogen), hCD45, hCD3, hCD4, hCD8, hCD69, hCD62L hCD45RO and hCD45RA (Biolegend). T cells state is determined by cell surface phenotypic markers: T memory stem cells (TSCM) CD45RO-CD62L⁺, Central memory T cells (TCM) CD45RO⁺CD62L⁺, effector memory T cells (TEM) CD45RO⁺CD62L⁻, terminal differentiate effector T cells (TEMRA) CD45RA⁺CD62L⁻. Data were acquired on an LSR Fortessa flow cytometer (BD Biosciences) and analyzed using FlowJo software Ver. Win64-10.6.1.

Three to ten days after PBMC injection (for humanization), mice were randomized into control and treatment groups and then either treated with control or with 1 mg/kg of TSLPR x CD3 bispecific antibody intraperitoneal (IP) once a week for three weeks. Bioluminescence imaging showed significantly less ALL cell bioluminescence signal after 21 days in mice injected with TSLPR x CD3 bispecific antibodies compared to mice injected with PBS (control) (FIG. 3).

Mice were also injected with patient-derived luciferase-labeled ALL blasts and donor PBMC (PBMC-875), treated with 0.01, 0.1, and 1 mg/kg of TSLPR x CD3 antibody and the percentage of live patient-derived ALL blasts was measured in the blood, bone marrow, and spleen of xenografted mice. Control mice were injected with PBS only or PBMC-875 cells and PBS. The percentage of ALL blasts was efficiently reduced over time in the blood of mice treated with 0.1 and 1 mg/kg of TSLPR x CD3 bispecific antibody (FIG. 4A). The percentage of ALL blasts was also reduced in the bone marrow and spleen at day 17 after treatment with 0.1 and 1 mg/kg of TSLPR x CD3 bispecific antibody (FIGS. 4B and 4C). Importantly, ALL blast growth inhibition in the bone marrow did not affect hematopoiesis and mice treated with TSLPR x CD3 bispecific antibody maintained normal hematopoiesis. T cell populations were also determined in the blood, bone marrow, and spleen of xenografted mice. Mice treated with 1 mg/kg TSLPR x CD3 bispecific antibody showed increases of blood CD3⁺ T cells from about 20% to about 70% from day 7 to day 17 after antibody treatment (FIG. 5A). CD3⁺ T cells in mice treated with 0.1 and 1 mg/kg also increased in the bone marrow (FIG. 5B) and spleen (FIG. 5C) compared to control mice injected with PBMC and PBS. These results demonstrated effective T cell expansion in human ALL xenografted mice treated with TSLPR x CD3 bispecific antibody.

### Example 8: Toxicokinetic Studies in Non-Human Primates

Female Macaca fascicularis were dosed by biweekly infusion with 0.3, 1, and 3 mg/kg of TSLPR x CD3 bispecific antibody and clinical study reports, toxicokinetics and neurological and behavioral evaluations were obtained. The serum levels of TSLPR x CD3 bispecific antibody pre-dose and 4 and 24 h after injection were determined and demonstrated that the Cₘₐₓ was substantially higher than the in vitro efficacious dose (FIG. 6). Further, a shorter half-life was observed in Non-Human Primates compared to mice, which could potentially be related to target mediated antibody clearance.

### Example 9: Cytokine Release Studies in Non-Human Primates

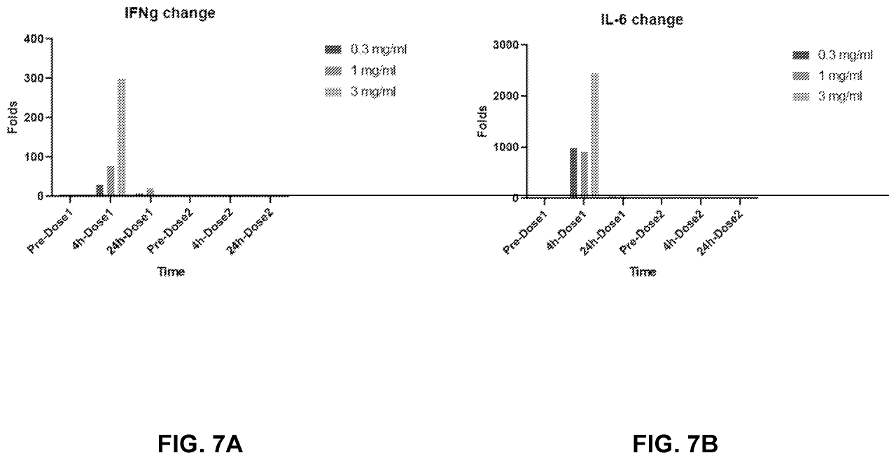

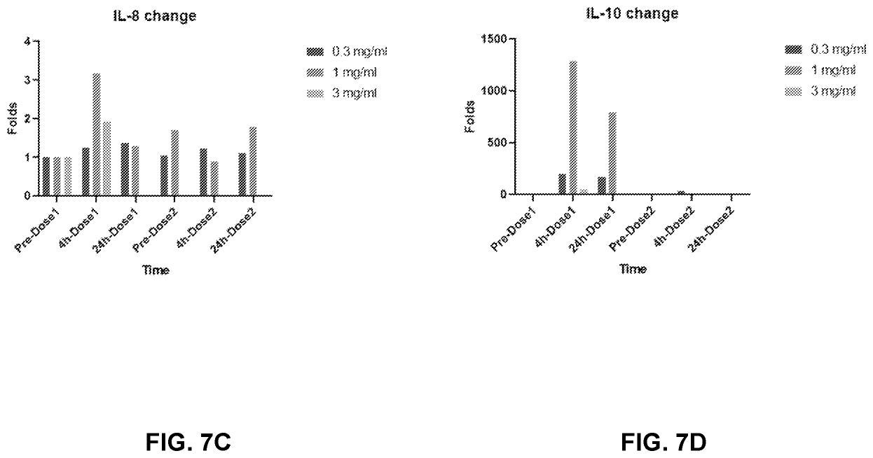

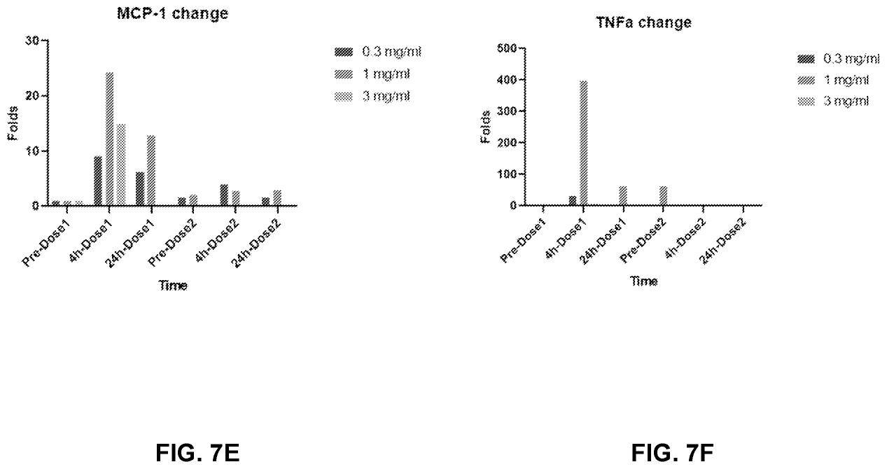

Cytokines were measured in the blood of Non-Human Primates treated with 0.3, 1, and 3 mg/kg of TSLPR x CD3 antibody pre-dose and 4 and 24 h after antibody injection. IFN-γ and IL-6 showed a substantial increase 4 h after injection of the highest dose (3 mg/kg) but not thereafter or after a second antibody dose (FIGS. 7A and 7B). IL-8 showed moderate increases at 1 mg/kg 4 h after first antibody dose (FIG. 7C), and IL-10 showed moderate increases at 1 mg/kg 4 h and 24 h after first antibody dose (FIG. 7D). MCP-1 showed increases at all three doses 4 h after first antibody injection and moderate increases at 0.3 and 1 mg/kg 24 h after first injection (FIG. 7E). TNF-α showed a substantial increase only at the 1 mg/kg dose 4 h after first antibody injection (FIG. 7F). Therefore, bispecific TSLPR x CD3 antibody in Non-Human Primates showed minimal or limited adverse events at 0.3 mg/kg, tolerable treatment associated toxicity at 1 mg/kg, and intolerable toxicity at 3 mg/kg. The maximum tolerated dose was determined to be ≤1 mg/kg.

### Example 10: Pharmacokinetics Studies in Mice

The study protocol was approved by MDACC after review by the Animal Care and Use Committee of the AAALAC-certified institution. Single dose PK of anti-TSLPR x anti-CD3 was studied in adult, female NSG (the Jackson Laboratory) mice as well as PBMC humanized NSG mice. For the PK in NSG mice, animals were randomized into treatment groups, and were ip. injected with the bispecific antibody at 0.1, 0.3 and 1 mg/kg (n=5). For PK in humanized NSG mice, 1×10⁷ PBMC were iv injected into the mice 10 days prior to randomization for dosing. Blood samples were collected at indicated time points post injection through serial sampling and processed to plasma for bioanalytical and PK analyses. Concentrations of anti-TSLPR x CD3 in plasma samples were measured using antigen coated ECL based ELISA (MSD, MD). PK analyses were performed according to standard non-compartmental analysis using WinNonlin package (v. 6.3, Pharsight, Certara Company).

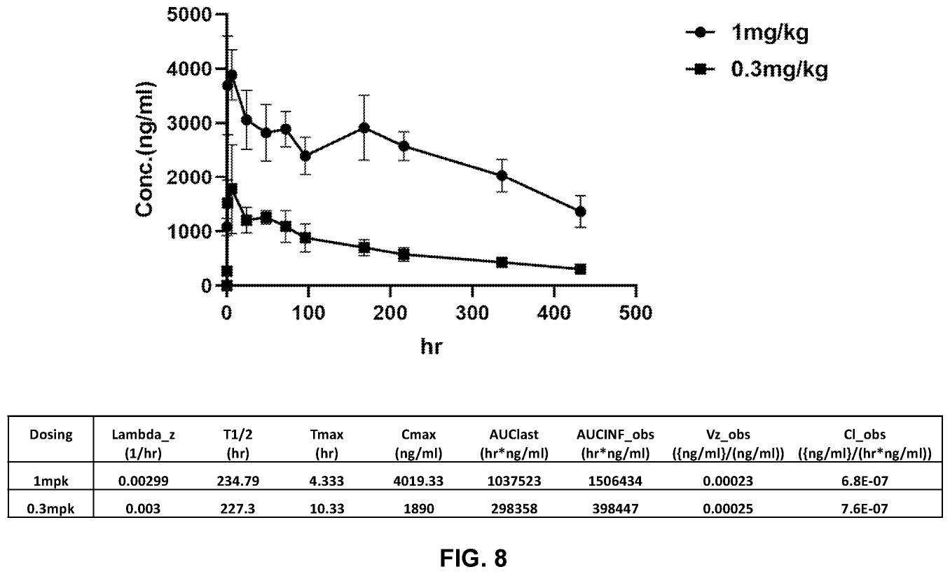

The PK studies revealed regular IgG pharmacokinetics of bispecific TSLPR x CD3 antibody (FIG. 8).

### Example 11: Engineering Additional Anti-TSLPR X Anti-CD3 Bispecific Antibodies

The above described TSLPR x CD3 bsAb (hereon designated as XENP39597) was produced in the 1+1 Fab-scFv-Fc format (cartoon schematic depicted in FIG. 23A) that comprises a single-chain Fv (“scFv”) covalently attached to a first heterodimeric Fc domain, a heavy chain variable region (VH) covalently attached to a complementary second heterodimeric Fc domain, and a light chain (LC) transfected separately so that a Fab domain is formed with the variable heavy domain.

Another format is the 2+1 Fab₂-scFv-Fc format (cartoon schematic depicted in FIG. 23B) that comprises a VH domain covalently attached to a CH1 domain covalently attached to an scFv covalently attached to a first heterodimeric Fc domain (VH-CH1-scFv-Fc), a VH domain covalently attached to a complementary second heterodimeric Fc domain, and a LC transfected separately so that Fab domains are formed with the VH domains.

While the above described TSLPR x CD3 bsAb utilized clone 1B7 variable regions, additional TSLPR binding domains can be used including those described in Tables 9 and 10. Further, while the above described TSLPR x CD3 bsAb utilized the CD3-High [VHVL] (a.k.a. [anti-CD3]_H1.30_L1.47_scFv, sequences for which are depicted in Figure X), additional CD3 binding domains including those described in FIG. 24 can be used.

Additionally, the TSLPR x CD3 bispecific antibodies of the invention may be engineered with M428L/N434S Fc variants for increased serum half-life.

DNA encoding chains of the TSLPR x CD3 bsAbs were generated by standard gene synthesis followed by isothermal cloning (Gibson assembly) or subcloning into a pTT5 expression vector containing fusion partners (e.g. domain linkers as depicted in FIG. 16 and/or backbones as depicted in FIGS. 19-22). DNA was transfected into HEK293E cells for expression. Sequences for illustrative TSLPR x CD3 bsAbs in the 1+1 Fab-scFv-Fc format and in the 2+1 Fab₂-scFv-Fc format and having various TSLPR and CD3 binding domains are depicted in FIGS. 24, Tables 3-10 and SEQ ID NOs: 1-228 and 331-396.

### Example 12: Characterizing Additional Anti-TSLPR X Anti-CD3 Bispecific Antibodies

The additional anti-TSLPR x anti-CD3 bispecific antibodies engineered with alternative CD3 binding domains and/or in the 2+1 Fab₂-scFv-Fc format (sequences for which are depicted in FIG. 26) were characterized and compared to the above described TSLPR x CD3 bsAb in the 1+1 Fab-scFv-Fc format comprising clone 1B7 and CD3-High [VHVL] XENP39597.

First, cell binding of XENP39597, XENP39599 (2+1; CD3-High-Int #1[VLVH]), and XENP39596 (bivalent mAb) to TSLPR″ cell lines were investigated. The antibodies were incubated at the indicated concentrations with 10,000 U266B1 and SNU423 cells on ice for an hour. Samples were then washed and stained with AlexaF647-labeled anti-Fc to detect binding. Data are depicted in FIG. 27.

Next, efficacy of the additional TSLPR x CD3 bispecific antibodies to induce TSLPR⁺ cell lysis (redirected T cell cytotoxicity or RTCC) was investigated in a number of experiments. In a first set of experiments, 20K U266B1 or SNU423 cells and incubated with 200K T cells (isolated from PBMC) with various concentrations of TSLPR x CD3 bispecific antibodies at 37° C. for 48 hours (RTCC data depicted in FIG. 28). In another set of experiments, 20K U266B1, MHH-CALL-4, or MUTZ5 cells were incubated with human PBMCs (effector:target 1:1) with various with various concentrations of TSLPR x CD3 bispecific antibodies at 37° C. for 24 hours (RTCC data depicted in FIG. 29). In another experiment investigating RTCC on TSLPR™ cells having various TSLPR cell densities, Reh cells (0.4K antigen density) were transfected to express various TSLPR densities (55K, 100K, and 160K) (in comparison to MUTZ5 having 10K antigen density) and incubated with T cells isolated from human PBMCs (1:1 effector:target) at 37° C. for 48 hours (RTCC data depicted in FIG. 30). In another experiment investigating the in vitro efficacy of XENP40361 having the lower affinity CD3-High-Int #2 binding domain, 50K MUTZ5 were incubated with T cells isolated from PBMCs (1:1 effector:target ratio) at 37° C. for 48 hours (RTCC data depicted in FIG. 31). In each of these experiments, % RTCC was determined by labelling target cells with CFSE Cell Proliferation Kit (Invitrogen) and determining % of dead CFSE+ cells using Live/Dead Fixable Blue Dead Cell Stain (Invitrogen). Antigen densities were estimated by FACS using fluorescently-labeled beads as advised by the QuickCal protocol (Bangs Laboratories, Inc., Fishers, IN). 50,000 cells per well and MESF beads were combined with A647-conjugated anti-TSLPR mAb for 30 minutes at 4° C. Cells were then washed and fixed in 1% PFA. Flow cytometry was performed to determine antibody binding, and antigen density calculations were carried out using QuickCal® V.2.3. software (Bangs Laboratories, Inc., Fishers, IN). To better compare XENP39597, XENP39598, and XENP39599, RTCC EC50 on various cell lines from the various experiments are depicted in the table in FIG. 32.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

What is claimed is:

1. A bispecific antibody comprising:
a) a thymic stromal lymphopoietin receptor (TSLPR) binding domain comprising i) a first variable heavy domain (VH1), and ii) a first variable light domain (VL1); and
b) a CD3 epsilon (CD3e) binding domain comprising i) a second variable heavy domain (VH2), and ii) a second variable light domain (VL2).

2. The bispecific antibody according to claim 1, wherein VH1 and VL1 are selected from one of the following:
(1) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(2) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:11 or a variant thereof; or
(3) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:12 or a variant thereof; or
(4) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:148 or a variant thereof; or
(5) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 149 or a variant thereof; or
(6) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 150 or a variant thereof; or
(7) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:151 or a variant thereof; or
(8) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:152 or a variant thereof; or
(9) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:153 or a variant thereof; or
(10) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154 or a variant thereof; or
(11) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 155 or a variant thereof; or
(12) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 156 or a variant thereof; or
(13) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(14) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:158 or a variant thereof; or
(15) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(16) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 160 or a variant thereof; or
(17) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:161 or a variant thereof; or
(18) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 162 or a variant thereof; or
(19) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 163 or a variant thereof; or
(20) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 164 or a variant thereof; or
(21) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof.

3. The bispecific antibody according to claim 2, wherein VH1 and VL1 are selected from the following:
(1) (i) a VH1 having an amino acid sequence of SEQ ID NO:7 or a variant thereof; and (ii) a VL1 having an amino acid sequence of SEQ ID NO:10 or a variant thereof; or
(2) (i) a VH1 having an amino acid sequence of SEQ ID NO:8 or a variant thereof; and (ii) a VL1 having an amino acid sequence of SEQ ID NO:11 or a variant thereof; or
(3) (i) a VH1 having an amino acid sequence of SEQ ID NO:9 or a variant thereof; and (ii) a VL1 having an amino acid sequence of SEQ ID NO: 12 or a variant thereof; or
(4) (i) a VH1 having an amino acid sequence of SEQ ID NO:76 or a variant thereof; and (ii) a VL1 having an amino acid sequence of SEQ ID NO:148 or a variant thereof; or
(5) (i) a VH1 having an amino acid sequence of SEQ ID NO:77 or a variant thereof; and (ii) a VL1 having an amino acid sequence of SEQ ID NO:149 or a variant thereof; or
(6) (i) a VH1 having an amino acid sequence of SEQ ID NO:78 or a variant thereof; and (ii) a VL1 having an amino acid sequence of SEQ ID NO:150 or a variant thereof; or
(7) (i) a VH1 having an amino acid sequence of SEQ ID NO:79 or a variant thereof; and (ii) a VL1 having an amino acid sequence of SEQ ID NO:151 or a variant thereof; or
(8) (i) a VH1 having an amino acid sequence of SEQ ID NO:80 or a variant thereof; and (ii) a VL1 having an amino acid sequence of SEQ ID NO: 152 or a variant thereof; or
(9) (i) a VH1 having an amino acid sequence of SEQ ID NO:81 or a variant thereof; and (ii) a VL1 having an amino acid sequence of SEQ ID NO:153 or a variant thereof; or
(10) (i) a VH1 having an amino acid sequence of SEQ ID NO:82 or a variant thereof; and (ii) a VL1 having an amino acid sequence of SEQ ID NO:154 or a variant thereof; or
(11) (i) a VH1 having an amino acid sequence of SEQ ID NO:83 or a variant thereof; and (ii) a VL1 having an amino acid sequence of SEQ ID NO:155 or a variant thereof; or
(12) (i) a VH1 having an amino acid sequence of SEQ ID NO:84 or a variant thereof; and (ii) a VL1 having an amino acid sequence of SEQ ID NO:156 or a variant thereof; or
(13) (i) a VH1 having an amino acid sequence of SEQ ID NO:85 or a variant thereof; and (ii) a VL1 having an amino acid sequence of SEQ ID NO:157 or a variant thereof; or
(14) (i) a VH1 having an amino acid sequence of SEQ ID NO:86 or a variant thereof; and (ii) a VL1 having an amino acid sequence of SEQ ID NO:158 or a variant thereof; or
(15) (i) a VH1 having an amino acid sequence of SEQ ID NO:87 or a variant thereof; and (ii) a VL1 having an amino acid sequence of SEQ ID NO:159 or a variant thereof; or
(16) (i) a VH1 having an amino acid sequence of SEQ ID NO:88 or a variant thereof; and (ii) a VL1 having an amino acid sequence of SEQ ID NO: 160 or a variant thereof; or
(17) (i) a VH1 having an amino acid sequence of SEQ ID NO:89 or a variant thereof; and (ii) a VL1 having an amino acid sequence of SEQ ID NO: 161 or a variant thereof; or
(18) (i) a VH1 having an amino acid sequence of SEQ ID NO:90 or a variant thereof; and (ii) a VL1 having an amino acid sequence of SEQ ID NO:162 or a variant thereof; or
(19) (i) a VH1 having an amino acid sequence of SEQ ID NO:91 or a variant thereof; and (ii) a VL1 having an amino acid sequence of SEQ ID NO: 163 or a variant thereof; or
(20) (i) a VH1 having an amino acid sequence of SEQ ID NO:92 or a variant thereof; and (ii) a VL1 having an amino acid sequence of SEQ ID NO:164 or a variant thereof; or
(21) (i) a VH1 having an amino acid sequence of SEQ ID NO:93 or a variant thereof; and (ii) a VL1 having an amino acid sequence of SEQ ID NO:165 or a variant thereof.

4. The bispecific antibody according to any one of claims 1 to 3, wherein VH2 and VL2 are selected from the following:
(1) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(2) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(3) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(4) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(5) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(6) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

5. The bispecific antibody according to claim 4, wherein VH2 and VL2 are selected from the following:
(1) (i) a VH2 having an amino acid sequence of SEQ ID NO:333 or a variant thereof; and (ii) a VL2 having an amino acid sequence of SEQ ID NO:337 or a variant thereof; or
(2) (i) a VH2 having an amino acid sequence of SEQ ID NO:344 or a variant thereof; and (ii) a VL2 having an amino acid sequence of SEQ ID NO:348 or a variant thereof; or
(3) (i) a VH2 having an amino acid sequence of SEQ ID NO:355 or a variant thereof; and (ii) a VL2 having an amino acid sequence of SEQ ID NO:359 or a variant thereof; or
(4) (i) a VH2 having an amino acid sequence of SEQ ID NO:366 or a variant thereof; and (ii) a VL2 having an amino acid sequence of SEQ ID NO:370 or a variant thereof; or
(5) (i) a VH2 having an amino acid sequence of SEQ ID NO:377 or a variant thereof; and (ii) a VL2 having an amino acid sequence of SEQ ID NO:381 or a variant thereof; or
(6) (i) a VH2 having an amino acid sequence of SEQ ID NO:388 or a variant thereof; and (ii) a VL2 having an amino acid sequence of SEQ ID NO:392 or a variant thereof.

6. The bispecific antibody according to any one of claims 1 to 5, wherein bispecific antibody further comprises a first Fc domain and a second Fc domain.

7. The bispecific antibody according to claim 6, wherein the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W:T366S/L368A/Y407V, wherein numbering is according to EU numbering.

8. The bispecific antibody according to claim 7, wherein the first and second Fc domains comprise heterodimerization skew variants S364K/E357Q: L368D/K370S, wherein numbering is according to EU numbering.

9. The bispecific antibody according to any one of claims 6 to 8, wherein the first and second Fc domains each comprise one or more ablation variants.

10. The bispecific antibody according to claim 9, wherein the one or more ablation variants comprise E233P/L234V/L235A/G236del/S267K, wherein numbering is according to EU numbering.

11. The bispecific antibody according to any of claims 6 to 10, wherein one of the first or second monomer further comprises one or more pI variants.

12. The bispecific antibody according to claim 11, wherein the pI variants N208D/Q295E/N384D/Q418E/N421D, wherein numbering is according to EU numbering.

13. A heterodimeric antibody comprising:
a) a first monomer comprising:
i) a single chain variable fragment (scFv); and
ii) a first Fc domain, wherein the scFv is covalently attached to the N-terminus of the first Fc domain using a domain linker;
b) a second monomer comprising, from N-terminal to C-terminal, a VH1-CH1-hinge-CH2-CH3, wherein VH1 is a first variable heavy domain and CH2-CH3 is a second Fc domain; and
c) a light chain comprising, from N-terminal to C-terminal, VL1-CL, wherein VL1 is a first variable light domain and CL is a constant light domain,
wherein the scFv comprises a second VH domain (VH2), a scFv linker, and a second variable light domain (VL2),
wherein the VH1 and the VL1 together form a first antigen binding domain (ABD) and the VH2 and the VL2 together form a second ABD, and
wherein one of the first ABD and second ABD is a thymic stromal lymphopoietin receptor (TSLPR) binding domain and the other of the first ABD and second ABD is a CD3 epsilon (CD3e) binding domain.

14. The heterodimeric antibody according to claim 13, wherein the scFv comprises, from N-terminal to C-terminal, VH2-scFv linker-VL2.

15. The heterodimeric antibody according to claim 13, wherein the scFv comprises, from N-terminal to C-terminal, VL2-scFv linker-VH2.

16. The heterodimeric antibody according to any one of claims 13 to 15, wherein the first ABD is the TSLPR binding domain and the second ABD is the CD3e binding domain.

17. The heterodimeric antibody according to claim 16, wherein VH1 and VL1 are selected from one of the following:
(1) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(2) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 11 or a variant thereof; or
(3) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 12 or a variant thereof; or
(4) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 148 or a variant thereof; or
(5) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 149 or a variant thereof; or
(6) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 150 or a variant thereof; or
(7) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 151 or a variant thereof; or
(8) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:152 or a variant thereof; or
(9) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:153 or a variant thereof; or
(10) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154 or a variant thereof; or
(11) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:155 or a variant thereof; or
(12) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:156 or a variant thereof; or
(13) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(14) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:158 or a variant thereof; or
(15) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(16) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 160 or a variant thereof; or
(17) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 161 or a variant thereof; or
(18) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:162 or a variant thereof; or
(19) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:163 or a variant thereof; or
(20) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:164 or a variant thereof; or
(21) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof.

18. The heterodimeric antibody according to claim 17, wherein VH1 and VL1 are selected from the following:
(1) (i) a VH1 having an amino acid sequence of SEQ ID NO:7 or a variant thereof; and (ii) a VL1 having an amino acid sequence of SEQ ID NO:10 or a variant thereof; or
(2) (i) a VH1 having an amino acid sequence of SEQ ID NO:8 or a variant thereof; and (ii) a VL1 having an amino acid sequence of SEQ ID NO:11 or a variant thereof; or
(3) (i) a VH1 having an amino acid sequence of SEQ ID NO:9 or a variant thereof; and (ii) a VL1 having an amino acid sequence of SEQ ID NO:12 or a variant thereof; or
(4) (i) a VH1 having an amino acid sequence of SEQ ID NO:76 or a variant thereof; and (ii) a VL1 having an amino acid sequence of SEQ ID NO:148 or a variant thereof; or
(5) (i) a VH1 having an amino acid sequence of SEQ ID NO:77 or a variant thereof; and (ii) a VL1 having an amino acid sequence of SEQ ID NO:149 or a variant thereof; or
(6) (i) a VH1 having an amino acid sequence of SEQ ID NO:78 or a variant thereof; and (ii) a VL1 having an amino acid sequence of SEQ ID NO: 150 or a variant thereof; or
(7) (i) a VH1 having an amino acid sequence of SEQ ID NO:79 or a variant thereof; and (ii) a VL1 having an amino acid sequence of SEQ ID NO:151 or a variant thereof; or
(8) (i) a VH1 having an amino acid sequence of SEQ ID NO:80 or a variant thereof; and (ii) a VL1 having an amino acid sequence of SEQ ID NO:152 or a variant thereof; or
(9) (i) a VH1 having an amino acid sequence of SEQ ID NO:81 or a variant thereof; and (ii) a VL1 having an amino acid sequence of SEQ ID NO:153 or a variant thereof; or
(10) (i) a VH1 having an amino acid sequence of SEQ ID NO:82 or a variant thereof; and (ii) a VL1 having an amino acid sequence of SEQ ID NO:154 or a variant thereof; or
(11) (i) a VH1 having an amino acid sequence of SEQ ID NO:83 or a variant thereof; and (ii) a VL1 having an amino acid sequence of SEQ ID NO:155 or a variant thereof; or
(12) (i) a VH1 having an amino acid sequence of SEQ ID NO:84 or a variant thereof; and (ii) a VL1 having an amino acid sequence of SEQ ID NO: 156 or a variant thereof; or
(13) (i) a VH1 having an amino acid sequence of SEQ ID NO:85 or a variant thereof; and (ii) a VL1 having an amino acid sequence of SEQ ID NO:157 or a variant thereof; or
(14) (i) a VH1 having an amino acid sequence of SEQ ID NO:86 or a variant thereof; and (ii) a VL1 having an amino acid sequence of SEQ ID NO:158 or a variant thereof; or
(15) (i) a VH1 having an amino acid sequence of SEQ ID NO:87 or a variant thereof; and (ii) a VL1 having an amino acid sequence of SEQ ID NO:159 or a variant thereof; or
(16) (i) a VH1 having an amino acid sequence of SEQ ID NO:88 or a variant thereof; and (ii) a VL1 having an amino acid sequence of SEQ ID NO:160 or a variant thereof; or
(17) (i) a VH1 having an amino acid sequence of SEQ ID NO:89 or a variant thereof; and (ii) a VL1 having an amino acid sequence of SEQ ID NO:161 or a variant thereof; or
(18) (i) a VH1 having an amino acid sequence of SEQ ID NO:90 or a variant thereof; and (ii) a VL1 having an amino acid sequence of SEQ ID NO: 162 or a variant thereof; or
(19) (i) a VH1 having an amino acid sequence of SEQ ID NO:91 or a variant thereof; and (ii) a VL1 having an amino acid sequence of SEQ ID NO:163 or a variant thereof; or
(20) (i) a VH1 having an amino acid sequence of SEQ ID NO:92 or a variant thereof; and (ii) a VL1 having an amino acid sequence of SEQ ID NO:164 or a variant thereof; or
(21) (i) a VH1 having an amino acid sequence of SEQ ID NO:93 or a variant thereof; and (ii) a VL1 having an amino acid sequence of SEQ ID NO:165 or a variant thereof.

19. The heterodimeric antibody according to any one of claims 16 to 18, wherein VH2 and VL2 are selected from one of the following:
(1) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(2) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(3) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(4) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(5) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(6) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

20. The heterodimeric antibody according to claim 19, where VH2 and VL2 are selected from one of the following:
(1) (i) a VH2 having an amino acid sequence of SEQ ID NO:333 or a variant thereof; and (ii) a VL2 having an amino acid sequence of SEQ ID NO:337 or a variant thereof; or
(2) (i) a VH2 having an amino acid sequence of SEQ ID NO:344 or a variant thereof; and (ii) a VL2 having an amino acid sequence of SEQ ID NO:348 or a variant thereof; or
(3) (i) a VH2 having an amino acid sequence of SEQ ID NO:355 or a variant thereof; and (ii) a VL2 having an amino acid sequence of SEQ ID NO:359 or a variant thereof; or
(4) (i) a VH2 having an amino acid sequence of SEQ ID NO:366 or a variant thereof; and (ii) a VL2 having an amino acid sequence of SEQ ID NO:370 or a variant thereof; or
(5) (i) a VH2 having an amino acid sequence of SEQ ID NO:377 or a variant thereof; and (ii) a VL2 having an amino acid sequence of SEQ ID NO:381 or a variant thereof; or
(6) (i) a VH2 having an amino acid sequence of SEQ ID NO:388 or a variant thereof; and (ii) a VL2 having an amino acid sequence of SEQ ID NO:392 or a variant thereof.

21. The heterodimeric antibody according to any one of claims 13 to 20, wherein the first Fc domain and second Fc domain are each variant Fc domains.

22. The heterodimeric antibody according to claim 21, wherein the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W:T366S/L368A/Y407V, wherein numbering is according to EU numbering.

23. The heterodimeric antibody according to claim 22, wherein the first and second Fc domains comprise heterodimerization skew variants S364K/E357Q: L368D/K370S, wherein numbering is according to EU numbering.

24. The heterodimeric antibody according to any one of claims 21 to 23, wherein the first and second Fc domains each comprise one or more ablation variants.

25. The heterodimeric antibody according to claim 24, wherein the one or more ablation variants comprise E233P/L234V/L235A/G236del/S267K, wherein numbering is according to EU numbering.

26. The heterodimeric antibody according to any of claims 21 to 25, wherein one of the first or second monomer further comprises one or more pI variants.

27. The heterodimeric antibody according to claim 26, wherein the CH1-hinge-CH2-CH3 of the second monomer comprises pI variants N208D/Q295E/N384D/Q418E/N421D, wherein numbering is according to EU numbering.

28. The heterodimeric antibody according to any of claims 21 to 27, wherein the CH1-hinge-CH2-CH3 of the second monomer comprises amino acid variants E233P/L234V/L235A/G236del/S267K/L368D/K370S/N208D/Q295E/N384D/Q418E/N421D,
wherein the first Fc domain comprises amino acid variants E233P/L234V/L235A/G236del/S267K/S364K/E357Q,
and wherein numbering is according to EU numbering.

29. The heterodimeric antibody according to any one of claims 22 to 28, wherein the first and second variant Fc domains each further comprise amino acid variants 428L/434S.

30. The heterodimeric antibody according to any one of claims 13 to 29, wherein the scFv linker is GKPGSGKPGSGKPGSGKPGS.

31. A heterodimeric antibody comprising:
a) a first monomer comprising, from N-terminal to C-terminal, VH1-CH1-first domain linker-scFv-second domain linker-CH2-CH3,
wherein VH1 is a first variable heavy domain, and CH2-CH3 is a first Fc domain;
b) a second monomer comprising, from N-terminal to C-terminal, a VH1-CH1-hinge-CH2-CH3, wherein CH2-CH3 is a second Fc domain; and
c) a light chain comprising, from N-terminal to C-terminal, VL1-CL, wherein VL1 is a first variable light domain and CL is a constant light domain,
wherein the scFv comprises a second VH domain (VH2), a scFv linker, and a second variable light domain (VL2),
wherein each of the VH1s and the VL1 together form a first antigen binding domain (ABD), and the VH2 and the VL2 form a second ABD, and
wherein one of the first ABDs and second ABD is a thymic stromal lymphopoietin receptor (TSLPR) binding domain and the other of the first ABDs and second ABD is a CD3 epsilon (CD3e) binding domain.

32. The heterodimeric antibody according to claim 31, wherein the scFv comprises, from N-terminal to C-terminal, VH2-scFv linker-VL2.

33. The heterodimeric antibody according to claim 31, wherein the scFv comprises, from N-terminal to C-terminal, VL2-scFv linker-VH2.

34. The heterodimeric antibody according to any one of claims 31 to 33, wherein the first ABDs are the TSLPR binding domains and the second ABD is the CD3e binding domain.

35. The heterodimeric antibody according to claim 34, wherein VH1s and VL1s are selected from one of the following:
(1) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:7 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 10 or a variant thereof; or
(2) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:8 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:11 or a variant thereof; or
(3) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:9 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 12 or a variant thereof; or
(4) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:76 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 148 or a variant thereof; or
(5) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:77 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 149 or a variant thereof; or
(6) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:78 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 150 or a variant thereof; or
(7) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:79 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:151 or a variant thereof; or
(8) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:80 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:152 or a variant thereof; or
(9) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:81 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 153 or a variant thereof; or
(10) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:82 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 154 or a variant thereof; or
(11) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:83 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:155 or a variant thereof; or
(12) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:84 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 156 or a variant thereof; or
(13) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:85 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 157 or a variant thereof; or
(14) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:86 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:158 or a variant thereof; or
(15) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:87 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 159 or a variant thereof; or
(16) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:88 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:160 or a variant thereof; or
(17) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:89 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 161 or a variant thereof; or
(18) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:90 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:162 or a variant thereof; or
(19) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:91 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 163 or a variant thereof; or
(20) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:92 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:164 or a variant thereof; or
(21) (i) a VH1 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:93 or a variant thereof; and (ii) a VL1 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO: 165 or a variant thereof.

36. The heterodimeric antibody according to claim 35, wherein VH1s and VL1s are selected from the following:
(1) (i) a VH1 having an amino acid sequence of SEQ ID NO:7 or a variant thereof; and (ii) a VL1 having an amino acid sequence of SEQ ID NO:10 or a variant thereof; or
(2) (i) a VH1 having an amino acid sequence of SEQ ID NO:8 or a variant thereof; and (ii) a VL1 having an amino acid sequence of SEQ ID NO:11 or a variant thereof; or
(3) (i) a VH1 having an amino acid sequence of SEQ ID NO:9 or a variant thereof; and (ii) a VL1 having an amino acid sequence of SEQ ID NO: 12 or a variant thereof; or
(4) (i) a VH1 having an amino acid sequence of SEQ ID NO:76 or a variant thereof; and (ii) a VL1 having an amino acid sequence of SEQ ID NO:148 or a variant thereof; or
(5) (i) a VH1 having an amino acid sequence of SEQ ID NO:77 or a variant thereof; and (ii) a VL1 having an amino acid sequence of SEQ ID NO:149 or a variant thereof; or
(6) (i) a VH1 having an amino acid sequence of SEQ ID NO:78 or a variant thereof; and (ii) a VL1 having an amino acid sequence of SEQ ID NO:150 or a variant thereof; or
(7) (i) a VH1 having an amino acid sequence of SEQ ID NO:79 or a variant thereof; and (ii) a VL1 having an amino acid sequence of SEQ ID NO:151 or a variant thereof; or
(8) (i) a VH1 having an amino acid sequence of SEQ ID NO:80 or a variant thereof; and (ii) a VL1 having an amino acid sequence of SEQ ID NO:152 or a variant thereof; or
(9) (i) a VH1 having an amino acid sequence of SEQ ID NO:81 or a variant thereof; and (ii) a VL1 having an amino acid sequence of SEQ ID NO: 153 or a variant thereof; or
(10) (i) a VH1 having an amino acid sequence of SEQ ID NO:82 or a variant thereof; and (ii) a VL1 having an amino acid sequence of SEQ ID NO:154 or a variant thereof; or
(11) (i) a VH1 having an amino acid sequence of SEQ ID NO:83 or a variant thereof; and (ii) a VL1 having an amino acid sequence of SEQ ID NO:155 or a variant thereof; or
(12) (i) a VH1 having an amino acid sequence of SEQ ID NO:84 or a variant thereof; and (ii) a VL1 having an amino acid sequence of SEQ ID NO:156 or a variant thereof; or
(13) (i) a VH1 having an amino acid sequence of SEQ ID NO:85 or a variant thereof; and (ii) a VL1 having an amino acid sequence of SEQ ID NO: 157 or a variant thereof; or
(14) (i) a VH1 having an amino acid sequence of SEQ ID NO:86 or a variant thereof; and (ii) a VL1 having an amino acid sequence of SEQ ID NO:158 or a variant thereof; or
(15) (i) a VH1 having an amino acid sequence of SEQ ID NO:87 or a variant thereof; and (ii) a VL1 having an amino acid sequence of SEQ ID NO: 159 or a variant thereof; or
(16) (i) a VH1 having an amino acid sequence of SEQ ID NO:88 or a variant thereof; and (ii) a VL1 having an amino acid sequence of SEQ ID NO:160 or a variant thereof; or
(17) (i) a VH1 having an amino acid sequence of SEQ ID NO:89 or a variant thereof; and (ii) a VL1 having an amino acid sequence of SEQ ID NO: 161 or a variant thereof; or
(18) (i) a VH1 having an amino acid sequence of SEQ ID NO:90 or a variant thereof; and (ii) a VL1 having an amino acid sequence of SEQ ID NO:162 or a variant thereof; or
(19) (i) a VH1 having an amino acid sequence of SEQ ID NO:91 or a variant thereof; and (ii) a VL1 having an amino acid sequence of SEQ ID NO: 163 or a variant thereof; or
(20) (i) a VH1 having an amino acid sequence of SEQ ID NO:92 or a variant thereof; and (ii) a VL1 having an amino acid sequence of SEQ ID NO:164 or a variant thereof; or
(21) (i) a VH1 having an amino acid sequence of SEQ ID NO:93 or a variant thereof; and (ii) a VL1 having an amino acid sequence of SEQ ID NO:165 or a variant thereof.

37. The heterodimeric antibody according to any one of claims 34 to 36, wherein VH2 and VL2 are selected from one of the following:
(1) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:333; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:337; or
(2) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:344; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:348; or
(3) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:355; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:359; or
(4) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:366; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:370; or
(5) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:377; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:381; or
(6) (i) a VH2 comprising a vhCDR1, a vhCDR2, and a vhCDR3 having an amino acid sequence of a vhCDR1, a vhCDR2, and a vhCDR3, respectively, of a VH having an amino acid sequence of SEQ ID NO:388; and (ii) a VL2 comprising a vlCDR1, a vlCDR2, and a vlCDR3 having an amino acid sequence of a vlCDR1, a vlCDR2, and a vlCDR3, respectively, of a VL having an amino acid sequence of SEQ ID NO:392.

38. The heterodimeric antibody according to claim 37, where VH2 and VL2 are selected from one of the following:
(1) (i) a VH2 having an amino acid sequence of SEQ ID NO:333 or a variant thereof; and (ii) a VL2 having an amino acid sequence of SEQ ID NO:337 or a variant thereof; or
(2) (i) a VH2 having an amino acid sequence of SEQ ID NO:344 or a variant thereof; and (ii) a VL2 having an amino acid sequence of SEQ ID NO:348 or a variant thereof; or
(3) (i) a VH2 having an amino acid sequence of SEQ ID NO:355 or a variant thereof; and (ii) a VL2 having an amino acid sequence of SEQ ID NO:359 or a variant thereof; or
(4) (i) a VH2 having an amino acid sequence of SEQ ID NO:366 or a variant thereof; and (ii) a VL2 having an amino acid sequence of SEQ ID NO:370 or a variant thereof; or
(5) (i) a VH2 having an amino acid sequence of SEQ ID NO:377 or a variant thereof; and (ii) a VL2 having an amino acid sequence of SEQ ID NO:381 or a variant thereof; or
(6) (i) a VH2 having an amino acid sequence of SEQ ID NO:388 or a variant thereof; and (ii) a VL2 having an amino acid sequence of SEQ ID NO:392 or a variant thereof.

39. The heterodimeric antibody according to any one of claims 31 to 38, wherein the first Fc domain and second Fc domain are each variant Fc domains.

40. The heterodimeric antibody according to claim 39, wherein the first and second Fc domains comprise a set of heterodimerization skew variants selected from the following heterodimerization variants: S364K/E357Q: L368D/K370S; S364K: L368D/K370S; S364K: L368E/K370S; D401K: T411E/K360E/Q362E; and T366W:T366S/L368A/Y407V, wherein numbering is according to EU numbering.

41. The heterodimeric antibody according to claim 40, wherein the first and second Fc domains comprise heterodimerization skew variants S364K/E357Q: L368D/K370S, wherein numbering is according to EU numbering.

42. The heterodimeric antibody according to any one of claims 39 to 41, wherein the first and second Fc domains each comprise one or more ablation variants.

43. The heterodimeric antibody according to claim 42, wherein the one or more ablation variants comprise E233P/L234V/L235A/G236del/S267K, wherein numbering is according to EU numbering.

44. The heterodimeric antibody according to any of claims 39 to 43, wherein one of the first or second monomer further comprises one or more pI variants.

45. The heterodimeric antibody according to claim 44, wherein the CH1-hinge-CH2-CH3 of the second monomer comprises pI variants N208D/Q295E/N384D/Q418E/N421D, wherein numbering is according to EU numbering.

46. The heterodimeric antibody according to any of claims 39 to 45, wherein the CH1-hinge-CH2-CH3 of the second monomer comprises amino acid variants E233P/L234V/L235A/G236del/S267K/L368D/K370S/N208D/Q295E/N384D/Q418E/N421 D,
wherein the first Fc domain comprises amino acid variants E233P/L234V/L235A/G236del/S267K/S364K/E357Q,
and wherein numbering is according to EU numbering.

47. The heterodimeric antibody according to any one of claims 40-46, wherein the first and second variant Fc domains each further comprise amino acid variants 428L/434S.

48. The heterodimeric antibody according to any one of claims 31 to 47, wherein the scFv linker is GKPGSGKPGSGKPGSGKPGS.

49. A nucleic acid composition comprising:
a) a first nucleic acid encoding the first variable heavy domain according to any one of claims 1-12;
b) a second nucleic acid encoding the first variable light domain according to any one of claims 1-12;
c) a third nucleic acid encoding the second variable heavy domain according to any one of claims 1-12; and
d) a fourth nucleic acid encoding the second variable heavy domain according to any one of claims 1-12;

50. A host cell comprising the nucleic acid composition of claim 49.

51. A method of making the bispecific antibody according to any of claims 1-12 comprising culturing the host cell of claim 50 under conditions wherein the bispecific antibody is expressed and recovering the heterodimeric antibody.

52. A nucleic acid composition comprising:
a) a first nucleic acid encoding the first monomer according to any of claims 13-48;
b) a second nucleic acid encoding the second monomer according to any of claims 13-48; and
c) a third nucleic acid encoding the light chain according to any of claims 13-48.

53. An expression vector composition comprising:
a) a first expression vector comprising the first nucleic acid of claim 52;
b) a second expression vector comprising the second nucleic acid of claim 52; and
c) a third expression vector comprising the third nucleic acid of claim 52; respectively.

54. A host cell comprising the expression vector composition of claim 53.

55. A method of making a heterodimeric antibody according to any of claims 13-48 comprising culturing the host cell of claim 54 under conditions wherein the heterodimeric antibody is expressed and recovering the heterodimeric antibody.

56. A method of treating a TSLPR-associated cancer in a patient in need thereof, comprising administering to the patient a bispecific antibody according to any of claims 1-12.

57. A method of treating a TSLPR-associated cancer in a patient in need thereof, comprising administering to the patient a bispecific antibody according to any of claims 13-48.

58. The method according to claim 56 or 57, wherein the TSLPR-associated cancer is a leukemia one of the following leukemias: an Acute Lymphoblastic Leukemia, a B-cell Acute Lymphoblastic Leukemia, or a Philadelphia chromosome-like B Cell Acute Lymphoblastic Leukemia.

59. An antibody or antigen-binding fragment thereof capable of binding to thymic stromal lymphopoietin receptor (TSLPR), wherein the antibody or antigen-binding fragment thereof comprises
a) a heavy chain variable region that comprises an amino acid sequence selected from the group consisting of SEQ ID NO: 13, 40, 41, 16, 64, 65, 19, 52, 53, and combinations thereof; an amino acid sequence selected from the group consisting of SEQ ID NO: 14, 42, 43, 17, 66, 67, 20, 54, 55, and combinations thereof; and an amino acid sequence selected from the group consisting of SEQ ID NO: 15, 44, 45, 18, 68, 69, 21, 56, 57, and combinations thereof; and
b) a light chain variable region that comprises an amino acid sequence selected from the group consisting of SEQ ID NO: 22, 46, 47, 25, 70, 71, 28, 58, 59 and combinations thereof; an amino acid sequence selected from the group consisting of SEQ ID NO: 23, 48, 49, 26, 72, 73, 29, 60, 61 and combinations thereof; and an amino acid sequence selected from the group consisting of SEQ ID NO: 24, 50, 51, 27, 74, 75, 30, 62, 63 and combinations thereof.

60. The antibody or antigen-binding fragment thereof of claim 59, wherein
a) the heavy chain variable region comprises an amino acid sequence having SEQ ID NO: 13, 14, and 15, and the light chain variable region comprises an amino acid sequence having SEQ ID NO: 22, 23, and 24;
b) the heavy chain variable region comprises an amino acid sequence having SEQ ID NO: 40, 42, and 44, and the light chain variable region comprises an amino acid sequence having SEQ ID NO: 46, 48, and 50;
c) the heavy chain variable region comprises an amino acid sequence having SEQ ID NO: 41, 43, and 45, and the light chain variable region comprises an amino acid sequence having SEQ ID NO: 47, 49, and 51;
d) the heavy chain variable region comprises an amino acid sequence having SEQ ID NO: 16, 17, and 18, and the light chain variable region comprises an amino acid sequence having SEQ ID NO: 25, 26, and 27;
e) the heavy chain variable region comprises an amino acid sequence having SEQ ID NO: 64, 66, and 68, and the light chain variable region comprises an amino acid sequence having SEQ ID NO: 70, 72, and 74;
f) the heavy chain variable region comprises an amino acid sequence having SEQ ID NO: 65, 67, and 69, and the light chain variable region comprises an amino acid sequence having SEQ ID NO: 71, 73, and 75;
g) the heavy chain variable region comprises an amino acid sequence having SEQ ID NO: 19, 20, and 21, and the light chain variable region comprises an amino acid sequence having SEQ ID NO: 28, 29, and 30;
h) the heavy chain variable region comprises an amino acid sequence having SEQ ID NO: 52, 54, and 56, and the light chain variable region comprises an amino acid sequence having SEQ ID NO: 58, 60, and 62; or
i) the heavy chain variable region comprises an amino acid sequence having SEQ ID NO: 53, 55, and 57, and the light chain variable region comprises an amino acid sequence having SEQ ID NO: 59, 61, and 63.

61. The antibody or antigen-binding fragment thereof of claim 59 or 60, comprising a heavy chain variable region (VH) and a light chain variable region (VL), wherein the VH comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 7 and the VL comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 10.

62. The antibody or antigen-binding fragment thereof of claim 59 or 60, comprising a heavy chain variable region (VH) and a light chain variable region (VL), wherein the VH comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 8 and the VL comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 11.

63. The antibody or antigen-binding fragment thereof of claim 59 or 60, comprising a heavy chain variable region (VH) and a light chain variable region (VL), wherein the VH comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 9 and the VL comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 12.

64. An antibody or antigen-binding fragment thereof capable of binding to TSLPR, wherein the antibody or antigen-binding fragment thereof comprises:
a) a CDR H1, CDR H2, and CDR H3 comprising the CDR H1, CDR H2, and CDR H3 amino acid sequences of any one of SEQ ID NO: 7, SEQ ID NO: 8, SEQ ID NO: 9, or 76-93, and
b) a CDR L1, CDR L2, and CDR L3 comprising the CDR L1, CDR L2, and CDR L3 amino acid sequences of any one of SEQ ID NO: 10, SEQ ID NO: 11, SEQ ID NO: 12, 148-165, or 227-229.

65. The antibody or antigen-binding fragment thereof of claim 64, wherein the CDRs are the Kabat-defined CDRs, the AbM-defined CDRs, or the IMGT-defined CDRs.

66. The antibody or antigen-binding fragment thereof of any one of claims 59 to 65, wherein the heavy chain variable region comprises an amino acid sequence that is at least 80% identical to SEQ ID NO: 7, SEQ ID NO: 8, or SEQ ID NO: 9.

67. The antibody or antigen-binding fragment thereof of any one of claims 59 to 66, wherein the heavy chain variable region comprises an amino acid sequence that is at least 85% identical to SEQ ID NO: 7, SEQ ID NO: 8, or SEQ ID NO: 9.

68. The antibody or antigen-binding fragment thereof of any one of claims 59 to 67, wherein the heavy chain variable region comprises an amino acid sequence that is at least 90% identical to SEQ ID NO: 7, SEQ ID NO: 8, or SEQ ID NO: 9.

69. The antibody or antigen-binding fragment thereof of any one of claims 59 to 68, wherein the heavy chain variable region comprises an amino acid sequence that is at least 95% identical to SEQ ID NO: 7, SEQ ID NO: 8, or SEQ ID NO: 9.

70. The antibody or antigen-binding fragment thereof of any one of claims 59 to 69, wherein the heavy chain variable region comprises an amino acid sequence that is at least 98% identical to SEQ ID NO: 7, SEQ ID NO: 8, or SEQ ID NO: 9.

71. The antibody or antigen-binding fragment thereof of any one of claims 59 to 70, wherein the heavy chain variable region comprises an amino acid sequence that is at least 99% identical to SEQ ID NO: 7, SEQ ID NO: 8, or SEQ ID NO: 9.

72. The antibody or antigen-binding fragment thereof of any one of claims 59 to 71, wherein the heavy chain variable region comprises an amino acid sequence having SEQ ID NO: 7, SEQ ID NO: 8, or SEQ ID NO: 9.

73. The antibody or antigen-binding fragment thereof of any one of claims 59 to 72, wherein the light chain variable region comprises an amino acid sequence that is at least 80% identical to SEQ ID NO: 10, SEQ ID NO: 11, or SEQ ID NO: 12

74. The antibody or antigen-binding fragment thereof of any one of claims 59 to 73, wherein the light chain variable region comprises an amino acid sequence that is at least 85% identical to SEQ ID NO: 10, SEQ ID NO: 11, or SEQ ID NO: 12.

75. The antibody or antigen-binding fragment thereof of any one of claims 59 to 74, wherein the light chain variable region comprises an amino acid sequence that is at least 90% identical to SEQ ID NO: 10, SEQ ID NO: 11, or SEQ ID NO: 12.

76. The antibody or antigen-binding fragment thereof of any one of claims 59 to 75, wherein the light chain variable region comprises an amino acid sequence that is at least 95% identical to SEQ ID NO: 10, SEQ ID NO: 11, or SEQ ID NO: 12.

77. The antibody or antigen-binding fragment thereof of any one of claims 59 to 76, wherein the light chain variable region comprises an amino acid sequence that is at least 98% identical to SEQ ID NO: 10, SEQ ID NO: 11, or SEQ ID NO: 12.

78. The antibody or antigen-binding fragment thereof of any one of claims 59 to 77, wherein the light chain variable region comprises an amino acid sequence that is at least 99% identical to SEQ ID NO: 10, SEQ ID NO: 11, or SEQ ID NO: 12.

79. The antibody or antigen-binding fragment thereof of any one of claims 59 to 78, wherein the light chain variable region comprises an amino acid sequence having SEQ ID NO: 10, SEQ ID NO: 11, or SEQ ID NO: 12.

80. The antibody or antigen-binding fragment thereof of any one of claims 59 to 79, wherein the heavy chain variable region and the light chain variable region form an antigen-binding site for a thymic stromal lymphopoietin receptor.

81. The antibody or antigen-binding fragment thereof of any one of claims 59 to 80, wherein the antibody or antigen-binding fragment thereof is human, humanized, or chimeric.

82. The antibody or antigen-binding fragment thereof of any one of claims 59 to 81, wherein the antibody or antigen-binding fragment thereof is an IgG antibody.

83. The antibody or antigen-binding fragment thereof of claim 82, wherein the IgG antibody is an IgG1 antibody or an IgG4 antibody.

84. The antibody or antigen-binding fragment thereof of any one of claims 59 to 83, wherein said antibody is an antigen-binding fragment of an antibody.

85. The antigen-binding fragment of claim 84, wherein said fragment is selected from the group consisting of Fab, F(ab′) 2, Fv, scFv, scFv-Fc, dsFv and a single domain molecule.

86. The antigen-binding fragment of claim 85, wherein said fragment is a scFv.

87. The antigen-binding fragment of claim 85, wherein said fragment is a Fab.

88. The antigen-binding fragment of claim 85, wherein said fragment is an intrabody.

89. The antibody or antigen-binding fragment thereof of any one of claim 59-81 or 84-88, wherein said antigen-binding fragment is devoid of an Fc region.

90. The antibody or antigen-binding fragment thereof of any one of claim 59-81 or 84-89, comprising a VH and a VL on the same polypeptide chain.

91. The antibody or antigen-binding fragment thereof of claim 90, wherein the VH and VL are connected by a linker.

92. The antibody or antigen-binding fragment thereof of any one of claims 59-91, wherein the antibody or antigen-binding fragment thereof is conjugated to an agent selected from the group consisting of a therapeutic agent, a prodrug, a peptide, a protein, an enzyme, a virus, a lipid, a biological response modifier, a pharmaceutical agent, and PEG.

93. A bispecific binding protein comprising three chains:
a. a first chain comprising, in order from the N-terminus to the C-terminus, a heavy chain variable region and a heavy chain constant region;
b. a second chain comprising, in order from the N-terminus to the C-terminus, a light chain variable region and a light chain constant region; and
c. a third chain comprising, in order from the N-terminus to the C-terminus, a single chain variable fragment (scFv) and a heavy chain constant region;
wherein the scFv of the third chain, in order from the N-terminus to the C-terminus, comprises a heavy chain variable region and a light chain variable region or, in order from the N-terminus to the C-terminus, comprises a light chain variable region and a heavy chain variable region; wherein the heavy chain variable region of the third chain comprises an amino acid sequence that comprises SEQ ID NO: 1, 2, and 3 and the light chain variable region of the third chain comprises an amino acid sequence that comprises SEQ ID NO: 4, 5, and 6;
wherein the heavy chain variable region of the first chain and the heavy chain variable region of the third chain comprise different amino acid sequences and the light chain variable region of the second chain and the light chain variable region of the scFv of the third chain comprise different amino acid sequences;
wherein the heavy chain variable region of the first chain comprises an amino acid sequence selected from the group consisting of SEQ ID NO: 13, 40, 41, 16, 64, 65, 19, 52, 53, and combinations thereof; an amino acid sequence selected from the group consisting of SEQ ID NO: 14, 42, 43, 17, 66, 67, 20, 54, 55, and combinations thereof; and an amino acid sequence selected from the group consisting of SEQ ID NO: 15, 44, 45, 18, 68, 69, 21, 56, 57, and combinations thereof;
wherein the light chain variable region of the second chain comprises an amino acid sequence selected from the group consisting of SEQ ID NO: 22, 46, 47, 25, 70, 71, 28, 58, 59 and combinations thereof; an amino acid sequence selected from the group consisting of SEQ ID NO: 23, 48, 49, 26, 72, 73, 29, 60, 61 and combinations thereof; and an amino acid sequence selected from the group consisting of SEQ ID NO: 24, 50, 51, 27, 74, 75, 30, 62, 63 and combinations thereof; and
wherein the heavy chain constant region of the first chain comprises an amino acid sequence that is at least 90% identical to the heavy chain constant region of the third chain and wherein the heavy chain constant region of the first chain and the heavy chain constant region of the third chain dimerize.

94. The bispecific binding protein of claim 93, wherein
a. the heavy chain variable region of the first chain comprises an amino acid sequence having SEQ ID NO: 13, 14, and 15, and the light chain variable region of the second chain comprises an amino acid sequence having SEQ ID NO: 22, 23, and 24;
b. the heavy chain variable region of the first chain comprises an amino acid sequence having SEQ ID NO: 40, 42, and 44, and the light chain variable region of the second chain comprises an amino acid sequence having SEQ ID NO: 46, 48, and 50;
c. the heavy chain variable region of the first chain comprises an amino acid sequence having SEQ ID NO: 41, 43, and 45, and the light chain variable region of the second chain comprises an amino acid sequence having SEQ ID NO: 47, 49, and 51;
d. the heavy chain variable region of the first chain comprises an amino acid sequence having SEQ ID NO: 16, 17, and 18, and the light chain variable region of the second chain comprises an amino acid sequence having SEQ ID NO: 25, 26, and 27;
e. the heavy chain variable region of the first chain comprises an amino acid sequence having SEQ ID NO: 64, 66, and 68, and the light chain variable region of the second chain comprises an amino acid sequence having SEQ ID NO: 70, 72, and 74;
f. the heavy chain variable region of the first chain comprises an amino acid sequence having SEQ ID NO: 65, 67, and 69, and the light chain variable region of the second chain comprises an amino acid sequence having SEQ ID NO: 71, 73, and 75;
g. the heavy chain variable region of the first chain comprises an amino acid sequence having SEQ ID NO: 19, 20, and 21, and the light chain variable region of the second chain comprises an amino acid sequence having SEQ ID NO: 28, 29, and 30;
h. the heavy chain variable region of the first chain comprises an amino acid sequence having SEQ ID NO: 52, 54, and 56, and the light chain variable region of the second chain comprises an amino acid sequence having SEQ ID NO: 58, 60, and 62; or
i. the heavy chain variable region of the first chain comprises an amino acid sequence having SEQ ID NO: 53, 55, and 57, and the light chain variable region of the second chain comprises an amino acid sequence having SEQ ID NO: 59, 61, and 63.

95. The bispecific binding protein of any one of claim 93 or 94, wherein the heavy chain constant region of the first chain comprises a CH1 domain and a Fc region comprising CH2 and CH3 domains and the heavy chain constant region of the third chain comprises a CH1 domain and a Fc region comprising CH2 and CH3 domains.

96. The bispecific binding protein of any one of claims 93 to 95, wherein the heavy chain constant region of the first chain comprises an amino acid sequence that is at least 95% identical to the heavy chain constant region of the third chain.

97. The bispecific binding protein of any one of claims 93 to 96, wherein the heavy chain constant region of the first chain comprises an amino acid sequence that is at least 97% identical to the heavy chain constant region of the third chain.

98. The bispecific binding protein of any one of claims 93 to 97, wherein the heavy chain constant region of the first chain and the heavy chain constant region of the third chain are subject to at least one modification to reduce the formation of a homodimer.

99. The bispecific binding protein of any one of claims 95 to 98, wherein the at least one modification comprises substituting an amino acid residue at the interface of the Fc region of the first chain with an amino acid residue having a side chain with an opposite charge to a residue at the interface of the Fc region of the third chain.

100. The bispecific binding protein of claim 99, wherein the amino acid at the interface of the Fc region of the first chain is selected from a glutamic acid, a serine, and an aspartic acid.

101. The bispecific binding protein of claim 99, wherein the amino acid at the interface of the Fc region of the third chain is selected from a glutamine, a lysine, a leucine, and an asparagine.

102. The bispecific binding protein of any one of claims 93 to 101, wherein the heavy chain variable region of the first chain comprises an amino acid sequence that is at least 80% identical to SEQ ID NO: 7, SEQ ID NO: 8, or SEQ ID NO: 9.

103. The bispecific binding protein of any one of claims 93 to 102, wherein the heavy chain variable region of the first chain comprises an amino acid sequence that is at least 85% identical to SEQ ID NO: 7, SEQ ID NO: 8, or SEQ ID NO: 9.

104. The bispecific binding protein of any one of claims 93 to 103, wherein the heavy chain variable region of the first chain comprises an amino acid sequence that is at least 90% identical to SEQ ID NO: 7, SEQ ID NO: 8, or SEQ ID NO: 9.

105. The bispecific binding protein of any one of claims 93 to 104, wherein the heavy chain variable region of the first chain comprises an amino acid sequence that is at least 95% identical to SEQ ID NO: 7, SEQ ID NO: 8, or SEQ ID NO: 9.

106. The bispecific binding protein of any one of claims 93 to 105, wherein the heavy chain variable region of the first chain comprises an amino acid sequence having SEQ ID NO: 7, SEQ ID NO: 8, or SEQ ID NO: 9.

107. The bispecific binding protein of any one of claims 93 to 106, wherein the light chain variable region of the second chain comprises an amino acid sequence that is at least 80% identical to SEQ ID NO: 10, SEQ ID NO: 11, or SEQ ID NO: 12

108. The bispecific binding protein of any one of claims 93 to 107, wherein the light chain variable region of the second chain comprises an amino acid sequence that is at least 85% identical to SEQ ID NO: 10, SEQ ID NO: 11, or SEQ ID NO: 12.

109. The bispecific binding protein of any one of claims 93 to 108, wherein the light chain variable region of the second chain comprises an amino acid sequence that is at least 90% identical to SEQ ID NO: 10, SEQ ID NO: 11, or SEQ ID NO: 12.

110. The bispecific binding protein of any one of claims 93 to 109, wherein the light chain variable region of the second chain comprises an amino acid sequence that is at least 95% identical to SEQ ID NO: 10, SEQ ID NO: 11, or SEQ ID NO: 12.

111. The bispecific binding protein of any one of claims 93 to 110, wherein the light chain variable region of the second chain comprises an amino acid sequence that having SEQ ID NO: 10, SEQ ID NO: 11, or SEQ ID NO: 12.

112. The bispecific binding protein of any one of claims 93 to 111, wherein the heavy chain variable region of the first chain and the light chain variable region of the second chain form an antigen-binding site for a thymic stromal lymphopoietin receptor.

113. The bispecific binding protein of any one of claims 95 to 112, wherein the heavy chain constant region of the first chain further comprises a hinge region provided between the CH1 domain and the Fc region.

114. The bispecific binding protein of any one of claims 95 to 113, wherein the heavy chain constant region of the third chain further comprises a hinge region provided between the CH1 domain and the Fc region.

115. The bispecific binding protein of claim 114, wherein an intermolecular disulfide bond is provided between the hinge region of the first chain and the hinge region of the third chain by introducing cysteine at corresponding positions between the CH3 domain of the Fc region of the first chain and the CH3 domain of the Fc region of the third chain.

116. An isolated polynucleotide comprising a nucleic acid molecule encoding the VH or heavy chain of the antibody or antigen-binding fragment thereof of any one of claims 59-92.

117. An isolated polynucleotide comprising a nucleic acid molecule encoding the VL or light chain of the antibody or antigen-binding fragment thereof of any one of claims 59-92.

118. An isolated polynucleotide comprising a nucleic acid molecule encoding the bispecific binding protein receptor of any one of claims 93-115.

119. An isolated vector comprising the polynucleotide of any one of claims 116-118.

120. A host cell comprising the polynucleotide of any one of claims 116-118 or the vector of claim 62.

121. The host cell of claim 120, which is selected form the group consisting of CHO, HEK-293T, HeLa and BHK cells, optionally wherein the CHO cell is a CHO-K1SP cell.

122. A method of producing an antibody or antigen-binding fragment thereof capable of binding to TSLPR, the method comprising:
(a) culturing the cell of claim 63 or 64 in a cell culture under conditions which allow expression of the antibody or antigen-binding fragment thereof; and
(b) recovering the antibody or antigen-binding fragment thereof from said cell culture.

123. An antibody or antigen-binding fragment thereof obtainable by the method of claim 65.

124. A pharmaceutical composition comprising the antibody or antigen-binding fragment thereof of any one of claim 59-92 or 123, the bispecific binding protein of any one of claims 93-115 or a vector of claim 119 and a pharmaceutically acceptable excipient.

125. A method of treating a cancer in a subject in need thereof, the method comprising administering to the subject an effective amount of antibody or antigen-binding fragment thereof of any one of claim 59-92 or 123, the bispecific binding protein of any one of claims 93-115, or a vector of claim 119 or the pharmaceutical composition of claim 124.

126. The method of claim 125, wherein the cancer is leukemia.

127. The method of claim 126, wherein the leukemia is an Acute Lymphoblastic Leukemia, a B-cell Acute Lymphoblastic Leukemia, or a Philadelphia chromosome-like B Cell Acute Lymphoblastic Leukemia.

128. The method of claim 127, wherein the cancer overexpresses TSLPR.

129. The method of any one of claims 68-71, wherein the method further comprises administering to the subject an effective amount of an additional cancer therapy.

130. The method of claim 129, wherein the additional cancer therapy is selected from radiation therapy, surgery, chemotherapy, gene therapy, DNA therapy, viral therapy, RNA therapy, immunotherapy, bone marrow transplantation, nanotherapy, monoclonal antibody therapy, and a combination thereof.

131. The method of any one of claims 125-130, wherein the method further comprises:
a. performing a chemiluminescence immunoassay (CLIA) on cells of the subject to quantify TSLPR expression;
b. performing a genetic analysis including genome and/or transcriptome profiling to quantify genes and/or transcripts of Ph like B-ALL; or
c. both a and b.

132. A formulation comprising the antibody or antigen-binding fragment thereof of any one of claim 59-92 or 123, the bispecific binding protein of any one of claims 93-115 or a vector of claim 119.

133. A kit comprising comprising the antibody or antigen-binding fragment thereof of any one of 59-92 or 123, the bispecific binding protein of any one of claims 93-115 or a vector of claim 119.
